SEQUENCE LISTING (16 sequences)

SEQ ID NO: 1 - DNA, 2316 bp, Yarrowia lipolytica
  misc_feature (1526)..(2316): partial coding sequence, including a 146 bp intron from nucleotide bases 1574-1719
gtgattgcctctgaatactttcaacaagttacacccttcgcggcgacgatctacagcccgatcacatgaactttggccgagggatgatgtaatcgagtatcgtggtagttcaatacgtacatgtacgatgggtgcctcaattgtgcgatactactacaagtgcagcacgctcgtgcccgtaccctactttgtcggacgtccctgctccctcgttcaacatctcaagctcaacaatcagtgttggacactgcaacgctagcagccggtacgtggctttagccccatgctccatgctccatgctccatgctctgggcctatgagctagccgtttggcgcacatagcatagtgacatgtcgatcaagtcaaagtcgaggtgtggaaaacgggctgcgggtcgccaggggcctcacaagcgcctccaccgcagacgcccacctcgttagcgtccattgcgatcgtctcggtacatttggttacattttgcgacaggttgaaatgaatcggccgacgctcggtagtcggaaagagccgggaccggccggcgagcataaaccggacgcagtaggatgtcctgcacgggtctttttgtggggtgtggagaaaggggtgcttggagatggaagccggtagaaccgggctgcttgtgcttggagatggaagccggtagaaccgggctgcttggggggatttggggccgctgggctccaaagaggggtaggcatttcgttggggttacgtaattgcggcatttgggtcctgcgcgcatgtcccattggtcagaattagtccggataggagacttatcagccaatcacagcgccggatccacctgtaggttgggttgggtgggagcacccctccacagagtagagtcaaacagcagcagcaacatgatagttgggggtgtgcgtgttaaaggaaaaaaaagaagcttgggttatattcccgctctatttagaggttgcgggatagacgccgacggagggcaatggcgccatggaaccttgcggatatcgatacgccgcggcggactgcgtccgaaccagctccagcagcgttttttccgggccattgagccgactgcgaccccgccaacgtgtcttggcccacgcactcatgtcatgttggtgttgggaggccactttttaagtagcacaaggcacctagctcgcagcaaggtgtccgaaccaaagaagcggctgcagtggtgcaaacggggcggaaacggcgggaaaaagccacgggggcacgaattgaggcacgccctcgaatttgagacgagtcacggccccattcgcccgcgcaatggctcgccaacgcccggtcttttgcaccacatcaggttaccccaagccaaacctttgtgttaaaaagcttaacatattataccgaacgtaggtttgggcgggcttgctccgtctgtccaaggcaacatttatataagggtctgcatcgccggctcaattgaatcttttttcttcttctcttctctatattcattcttgaattaaacacacatcaacatggccatcaaagtcggtattaacggattcgggcgaatcggacgaattgtgagtaccatagaaggtgatggaaacatgacccaacagaaacagatgacaagtgtcatcgacccaccagagcccaattgagctcatactaacagtcgacaacctgtcgaaccaattgatgactccccgacaatgtactaacacaggtcctgcgaaacgctctcaagaaccctgaggtcgaggtcgtcgctgtgaacgaccccttcatcgacaccgagtacgctgcttacatgttcaagtacgactccacccacggccgattcaagggcaaggtcgaggccaaggacggcggtctgatcatcgacggcaagcacatccaggtcttcggtgagcgagacccctccaacatcccctggggtaaggccggtgccgactacgttgtcgagtccaccggtgtcttcaccggcaaggaggctgcctccgcccacctcaagggtggtgccaagaaggtcatcatctccgccccctccggtgacgcccccatgttcgttgtcggtgtcaacctcgacgcctacaagcccgacatgaccgtcatctccaacgcttcttgtaccaccaactgtctggctccccttgccaaggttgtcaacgacaagtacggaatcattgagggtctcatgaccaccgtccactccatcaccgccacccagaagaccgttgacggtccttcccacaaggactggcgaggtggccgaaccgcctctggtaacatcatcccctcttccaccggagccgccaaggct

SEQ ID NO: 2 - DNA, 971 bp, Yarrowia lipolytica
gacgcagtaggatgtcctgcacgggtctttttgtggggtgtggagaaaggggtgcttggagatggaagccggtagaaccgggctgcttgtgcttggagatggaagccggtagaaccgggctgcttggggggatttggggccgctgggctccaaagaggggtaggcatttcgttggggttacgtaattgcggcatttgggtcctgcgcgcatgtcccattggtcagaattagtccggataggagacttatcagccaatcacagcgccggatccacctgtaggttgggttgggtgggagcacccctccacagagtagagtcaaacagcagcagcaacatgatagttgggggtgtgcgtgttaaaggaaaaaaaagaagcttgggttatattcccgctctatttagaggttgcgggatagacgccgacggagggcaatggcgccatggaaccttgcggatatcgatacgccgcggcggactgcgtccgaaccagctccagcagcgttttttccgggccattgagccgactgcgaccccgccaacgtgtcttggcccacgcactcatgtcatgttggtgttgggaggccactttttaagtagcacaaggcacctagctcgcagcaaggtgtccgaaccaaagaagcggctgcagtggtgcaaacggggcggaaacggcgggaaaaagccacgggggcacgaattgaggcacgccctcgaatttgagacgagtcacggccccattcgcccgcgcaatggctcgccaacgcccggtcttttgcaccacatcaggttaccccaagccaaacctttgtgttaaaaagcttaacatattataccgaacgtaggtttgggcgggcttgctccgtctgtccaaggcaacatttatataagggtctgcatcgccggctcaattgaatcttttttcttcttctcttctctatattcattcttgaattaaacacacatcaacatg

SEQ ID NO: 3 - DNA, 969 bp, Yarrowia lipolytica
gacgcagtaggatgtcctgcacgggtctttttgtggggtgtggagaaaggggtgcttggagatggaagccggtagaaccgggctgcttgtgcttggagatggaagccggtagaaccgggctgcttggggggatttggggccgctgggctccaaagaggggtaggcatttcgttggggttacgtaattgcggcatttgggtcctgcgcgcatgtcccattggtcagaattagtccggataggagacttatcagccaatcacagcgccggatccacctgtaggttgggttgggtgggagcacccctccacagagtagagtcaaacagcagcagcaacatgatagttgggggtgtgcgtgttaaaggaaaaaaaagaagcttgggttatattcccgctctatttagaggttgcgggatagacgccgacggagggcaatggcgccatggaaccttgcggatatcgatacgccgcggcggactgcgtccgaaccagctccagcagcgttttttccgggccattgagccgactgcgaccccgccaacgtgtcttggcccacgcactcatgtcatgttggtgttgggaggccactttttaagtagcacaaggcacctagctcgcagcaaggtgtccgaaccaaagaagcggctgcagtggtgcaaacggggcggaaacggcgggaaaaagccacgggggcacgaattgaggcacgccctcgaatttgagacgagtcacggccccattcgcccgcgcaatggctcgccaacgcccggtcttttgcaccacatcaggttaccccaagccaaacctttgtgttaaaaagcttaacatattataccgaacgtaggtttgggcgggcttgctccgtctgtccaaggcaacatttatataagggtctgcatcgccggctcaattgaatcttttttcttcttctcttctctatattcattcttgaattaaacacacatcaacc

SEQ ID NO: 4 - DNA, 9469 bp, Artificial Sequence
  Plasmid pYZGDG
ggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgatggtgtcgataagcttgatatcgaattcatgtcacacaaaccgatcttcgcctcaaggaaacctaattctacatccgagagactgccgagatccagtctacactgattaattttcgggccaataatttaaaaaaatcgtgttatataatattatatgtattatatatatacatcatgatgatactgacagtcatgtcccattgctaaatagacagactccatctgccgcctccaactgatgttctcaatatttaaggggtcatctcgcattgtttaataataaacagactccatctaccgcctccaaatgatgttctcaaaatatattgtatgaacttatttttattacttagtattattagacaacttacttgctttatgaaaaacacttcctatttaggaaacaatttataatggcagttcgttcatttaacaatttatgtagaataaatgttataaatgcgtatgggaaatcttaaatatggatagcataaatgatatctgcattgcctaattcgaaatcaacagcaacgaaaaaaatcccttgtacaacataaatagtcatcgagaaatatcaactatcaaagaacagctattcacacgttactattgagattattattggacgagaatcacacactcaactgtctttctctcttctagaaatacaggtacaagtatgtactattctcattgttcatacttctagtcatttcatcccacatattccttggatttctctccaatgaatgacattctatcttgcaaattcaacaattataataagatataccaaagtagcggtatagtggcaatcaaaaagcttctctggtgtgcttctcgtatttatttttattctaatgatccattaaaggtatatatttatttcttgttatataatccttttgtttattacatgggctggatacataaaggtattttgatttaattttttgcttaaattcaatcccccctcgttcagtgtcaactgtaatggtaggaaattaccatacttttgaagaagcaaaaaaaatgaaagaaaaaaaaaatcgtatttccaggttagacgttccgcagaatctagaatgcggtatgcggtacattgttcttcgaacgtaaaagttgcgctccctgagatattgtacatttttgcttttacaagtacaagtacatcgtacaactatgtactactgttgatgcatccacaacagtttgttttgtttttttttgttttttttttttctaatgattcattaccgctatgtatacctacttgtacttgtagtaagccgggttattggcgttcaattaatcatagacttatgaatctgcacggtgtgcgctgcgagttacttttagcttatgcatgctacttgggtgtaatattgggatctgttcggaaatcaacggatgctcaaccgatttcgacagtaataatttgaatcgaatcggagcctaaaatgaacccgagtatatctcataaaattctcggtgagaggtctgtgactgtcagtacaaggtgccttcattatgccctcaaccttaccatacctcactgaatgtagtgtacctctaaaaatgaaatacagtgccaaaagccaaggcactgagctcgtctaacggacttgatatacaaccaattaaaacaaatgaaaagaaatacagttctttgtatcatttgtaacaattaccctgtacaaactaaggtattgaaatcccacaatattcccaaagtccacccctttccaaattgtcatgcctacaactcatataccaagcactaacctaccaaacaccactaaaaccccacaaaatatatcttaccgaatatacagtaacaagctaccaccacactcgttgggtgcagtcgccagcttaaagatatctatccacatcagccacaactcccttcctttaataaaccgactacacccttggctattgaggttatgagtgaatatactgtagacaagacactttcaagaagactgtttccaaaacgtaccactgtcctccactacaaacacacccaatctgcttcttctagtcaaggttgctacaccggtaaattataaatcatcatttcattagcagggcagggccctttttatagagtcttatacactagcggaccctgccggtagaccaacccgcaggcgcgtcagtttgctccttccatcaatgcgtcgtagaaacgacttactccttcttgagcagctccttgaccttgttggcaacaagtctccgacctcggaggtggaggaagagcctccgatatcggcggtagtgataccagcctcgacggactccttgacggcagcctcaacagcgtcaccggcgggcttcatgttaagagagaacttgagcatcatggcggcagacagaatggtggcaatggggttgaccttctgcttgccgagatcgggggcagatccgtgacagggctcgtacagaccgaacgcctcgttggtgtcgggcagagaagccagagaggcggagggcagcagacccagagaaccggggatgacggaggcctcgtcggagatgatatcgccaaacatgttggtggtgatgatgataccattcatcttggagggctgcttgatgaggatcatggcggccgagtcgatcagctggtggttgagctcgagctgggggaattcgtccttgaggactcgagtgacagtctttcgccaaagtcgagaggaggccagcacgttggccttgtcaagagaccacacgggaagaggggggttgtgctgaagggccaggaaggcggccattcgggcaattcgctcaacctcaggaacggagtaggtctcggtgtcggaagcgacgccagatccgtcatcctcctttcgctctccaaagtagatacctccgacgagctctcggacaatgatgaagtcggtgccctcaacgtttcggatgggggagagatcggcgagcttgggcgacagcagctggcagggtcgcaggttggcgtacaggttcaggtcctttcgcagcttgaggagaccctgctcgggtcgcacgtcggttcgtccgtcgggagtggtccatacggtgttggcagcgcctccgacagcaccgagcataatagagtcagcctttcggcagatgtcgagagtagcgtcggtgatgggctcgccctccttctcaatggcagctcctccaatgagtcggtcctcaaacacaaactcggtgccggaggcctcagcaacagacttgagcaccttgacggcctcggcaatcacctcggggccacagaagtcgccgccgagaagaacaatcttcttggagtcagtcttggtcttcttagtttcgggttccattgtggatgtgtgtggttgtatgtgtgatgtggtgtgtggagtgaaaatctgtggctggcaaacgctcttgtatatatacgcacttttgcccgtgctatgtggaagactaaacctccgaagattgtgactcaggtagtgcggtatcggctagggacccaaaccttgtcgatgccgatagcgctatcgaacgtaccccagccggccgggagtatgtcggaggggacatacgagatcgtcaagggtttgtggccaactggtaaataaatgatgtcgacgcagtaggatgtcctgcacgggtctttttgtggggtgtggagaaaggggtgcttggagatggaagccggtagaaccgggctgcttgtgcttggagatggaagccggtagaaccgggctgcttggggggatttggggccgctgggctccaaagaggggtaggcatttcgttggggttacgtaattgcggcatttgggtcctgcgcgcatgtcccattggtcagaattagtccggataggagacttatcagccaatcacagcgccggatccacctgtaggttgggttgggtgggagcacccctccacagagtagagtcaaacagcagcagcaacatgatagttgggggtgtgcgtgttaaaggaaaaaaaagaagcttgggttatattcccgctctatttagaggttgcgggatagacgccgacggagggcaatggcgccatggaaccttgcggatatcgatacgccgcggcggactgcgtccgaaccagctccagcagcgttttttccgggccattgagccgactgcgaccccgccaacgtgtcttggcccacgcactcatgtcatgttggtgttgggaggccactttttaagtagcacaaggcacctagctcgcagcaaggtgtccgaaccaaagaagcggctgcagtggtgcaaacggggcggaaacggcgggaaaaagccacgggggcacgaattgaggcacgccctcgaatttgagacgagtcacggccccattcgcccgcgcaatggctcgccaacgcccggtcttttgcaccacatcaggttaccccaagccaaacctttgtgttaaaaagcttaacatattataccgaacgtaggtttgggcgggcttgctccgtctgtccaaggcaacatttatataagggtctgcatcgccggctcaattgaatcttttttcttcttctcttctctatattcattcttgaattaaacacacatcaaccatggatggtacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccgggatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgattaattaactagagcggccgccaccgcggcccgagattccggcctcttcggccgccaagcgacccgggtggacgtctagaggtacctagcaattaacagatagtttgccggtgataattctcttaacctcccacactcctttgacataacgatttatgtaacgaaactgaaatttgaccagatattgtgtccgc

SEQ ID NO: 5 - DNA, 969 bp, Yarrowia lipolytica
gacgcagtaggatgtcctgcacgggtctttttgtggggtgtggagaaaggggtgcttggagatggaagccggtagaaccgggctgcttgtgcttggagatggaagccggtagaaccgggctgcttggggggatttggggccgctgggctccaaagaggggtaggcatttcgttggggttacgtaattgcggcatttgggtcctgcgcgcatgtcccattggtcagaattagtccggataggagacttatcagccaatcacagcgccggatccacctgtaggttgggttgggtgggagcacccctccacagagtagagtcaaacagcagcagcaacatgatagttgggggtgtgcgtgttaaaggaaaaaaaagaagcttgggttatattcccgctctatttagaggttgcgggatagacgccgacggagggcaatggcgctatggaaccttgcggatatccatacgccgcggcggactgcgtccgaaccagctccagcagcgttttttccgggccattgagccgactgcgaccccgccaacgtgtcttggcccacgcactcatgtcatgttggtgttgggaggccactttttaagtagcacaaggcacctagctcgcagcaaggtgtccgaaccaaagaagcggctgcagtggtgcaaacggggcggaaacggcgggaaaaagccacgggggcacgaattgaggcacgccctcgaatttgagacgagtcacggccccattcgcccgcgcaatggctcgccaacgcccggtcttttgcaccacatcaggttaccccaagccaaacctttgtgttaaaaagcttaacatattataccgaacgtaggtttgggcgggcttgctccgtctgtccaaggcaacatttatataagggtctgcatcgccggctcaattgaatcttttttcttcttctcttctctatattcattcttgaattaaacacacatcaacc

SEQ ID NO: 6 - DNA, 971 bp, Yarrowia lipolytica
gacgcagtaggatgtcctgcacgggtctttttgtggggtgtggagaaaggggtgcttggatcgatggaagccggtagaaccgggctgcttgtgcttggagatggaagccggtagaaccgggctgcttggggggatttggggccgctgggctccaaagaggggtaggcatttcgttggggttacgtaattgcggcatttgggtcctgcgcgcatgtcccattggtcagaattagtccggataggagacttatcagccaatcacagcgccggatccacctgtaggttgggttgggtgggagcacccctccacagagtagagtcaaacagcagcagcaacatgatagttgggggtgtgcgtgttaaaggaaaaaaaagaagcttgggttatattcccgctctatttagaggttgcgggatagacgccgacggagggcaatggcgctatggaaccttgcggatatccatacgccgcggcggactgcgtccgaaccagctccagcagcgttttttccgggccattgagccgactgcgaccccgccaacgtgtcttggcccacgcactcatgtcatgttggtgttgggaggccactttttaagtagcacaaggcacctagctcgcagcaaggtgtccgaaccaaagaagcggctgcagtggtgcaaacggggcggaaacggcgggaaaaagccacgggggcacgaattgaggcacgccctcgaatttgagacgagtcacggccccattcgcccgcgcaatggctcgccaacgcccggtcttttgcaccacatcaggttaccccaagccaaacctttgtgttaaaaagcttaacatattataccgaacgtaggtttgggcgggcttgctccgtctgtccaaggcaacatttatataagggtctgcatcgccggctcaattgaatcttttttcttcttctcttctctatattcattcttgaattaaacacacatcaacc

SEQ ID NO: 7 - DNA, 909 bp, Yarrowia lipolytica
gatggaagccggtagaaccgggctgcttgtgcttggagatggaagccggtagaaccgggctgcttggggggatttggggccgctgggctccaaagaggggtaggcatttcgttggggttacgtaattgcggcatttgggtcctgcgcgcatgtcccattggtcagaattagtccggataggagacttatcagccaatcacagcgccggatccacctgtaggttgggttgggtgggagcacccctccacagagtagagtcaaacagcagcagcaacatgatagttgggggtgtgcgtgttaaaggaaaaaaaagaagcttgggttatattcccgctctatttagaggttgcgggatagacgccgacggagggcaatggcgctatggaaccttgcggatatccatacgccgcggcggactgcgtccgaaccagctccagcagcgttttttccgggccattgagccgactgcgaccccgccaacgtgtcttggcccacgcactcatgtcatgttggtgttgggaggccactttttaagtagcacaaggcacctagctcgcagcaaggtgtccgaaccaaagaagcggctgcagtggtgcaaacggggcggaaacggcgggaaaaagccacgggggcacgaattgaggcacgccctcgaatttgagacgagtcacggccccattcgcccgcgcaatggctcgccaacgcccggtcttttgcaccacatcaggttaccccaagccaaacctttgtgttaaaaagcttaacatattataccgaacgtaggtttgggcgggcttgctccgtctgtccaaggcaacatttatataagggtctgcatcgccggctcaattgaatcttttttcttcttctcttctctatattcattcttgaattaaacacacatcaacc

SEQ ID NO: 8 - DNA, 8600 bp, Artificial Sequence
  Plasmid pYZDE1SB
ggtggagctccagcttttgttccctttagtgagggttaatttcgagcttggcgtaatcatggtcatagctgtttcctgtgtgaaattgttatccgctcacaattccacacaacgtacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgatggtgtcgataagcttgatatcgaattcatgtcacacaaaccgatcttcgcctcaaggaaacctaattctacatccgagagactgccgagatccagtctacactgattaattttcgggccaataatttaaaaaaatcgtgttatataatattatatgtattatatatatacatcatgatgatactgacagtcatgtcccattgctaaatagacagactccatctgccgcctccaactgatgttctcaatatttaaggggtcatctcgcattgtttaataataaacagactccatctaccgcctccaaatgatgttctcaaaatatattgtatgaacttatttttattacttagtattattagacaacttacttgctttatgaaaaacacttcctatttaggaaacaatttataatggcagttcgttcatttaacaatttatgtagaataaatgttataaatgcgtatgggaaatcttaaatatggatagcataaatgatatctgcattgcctaattcgaaatcaacagcaacgaaaaaaatcccttgtacaacataaatagtcatcgagaaatatcaactatcaaagaacagctattcacacgttactattgagattattattggacgagaatcacacactcaactgtctttctctcttctagaaatacaggtacaagtatgtactattctcattgttcatacttctagtcatttcatcccacatattccttggatttctctccaatgaatgacattctatcttgcaaattcaacaattataataagatataccaaagtagcggtatagtggcaatcaaaaagcttctctggtgtgcttctcgtatttatttttattctaatgatccattaaaggtatatatttatttcttgttatataatccttttgtttattacatgggctggatacataaaggtattttgatttaattttttgcttaaattcaatcccccctcgttcagtgtcaactgtaatggtaggaaattaccatacttttgaagaagcaaaaaaaatgaaagaaaaaaaaaatcgtatttccaggttagacgttccgcagaatctagaatgcggtatgcggtacattgttcttcgaacgtaaaagttgcgctccctgagatattgtacatttttgcttttacaagtacaagtacatcgtacaactatgtactactgttgatgcatccacaacagtttgttttgtttttttttgttttttttttttctaatgattcattaccgctatgtatacctacttgtacttgtagtaagccgggttattggcgttcaattaatcatagacttatgaatctgcacggtgtgcgctgcgagttacttttagcttatgcatgctacttgggtgtaatattgggatctgttcggaaatcaacggatgctcaaccgatttcgacagtaataatttgaatcgaatcggagcctaaaatgaacccgagtatatctcataaaattctcggtgagaggtctgtgactgtcagtacaaggtgccttcattatgccctcaaccttaccatacctcactgaatgtagtgtacctctaaaaatgaaatacagtgccaaaagccaaggcactgagctcgtctaacggacttgatatacaaccaattaaaacaaatgaaaagaaatacagttctttgtatcatttgtaacaattaccctgtacaaactaaggtattgaaatcccacaatattcccaaagtccacccctttccaaattgtcatgcctacaactcatataccaagcactaacctaccaaacaccactaaaaccccacaaaatatatcttaccgaatatacagtaacaagctaccaccacactcgttgggtgcagtcgccagcttaaagatatctatccacatcagccacaactcccttcctttaataaaccgactacacccttggctattgaggttatgagtgaatatactgtagacaagacactttcaagaagactgtttccaaaacgtaccactgtcctccactacaaacacacccaatctgcttcttctagtcaaggttgctacaccggtaaattataaatcatcatttcattagcagggcagggccctttttatagagtcttatacactagcggaccctgccggtagaccaacccgcaggcgcgtcagtttgctccttccatcaatgcgtcgtagaaacgacttactccttcttgagcagctccttgaccttgttggcaacaagtctccgacctcggaggtggaggaagagcctccgatatcggcggtagtgataccagcctcgacggactccttgacggcagcctcaacagcgtcaccggcgggcttcatgttaagagagaacttgagcatcatggcggcagacagaatggtggcaatggggttgaccttctgcttgccgagatcgggggcagatccgtgacagggctcgtacagaccgaacgcctcgttggtgtcgggcagagaagccagagaggcggagggcagcagacccagagaaccggggatgacggaggcctcgtcggagatgatatcgccaaacatgttggtggtgatgatgataccattcatcttggagggctgcttgatgaggatcatggcggccgagtcgatcagctggtggttgagctcgagctgggggaattcgtccttgaggactcgagtgacagtctttcgccaaagtcgagaggaggccagcacgttggccttgtcaagagaccacacgggaagaggggggttgtgctgaagggccaggaaggcggccattcgggcaattcgctcaacctcaggaacggagtaggtctcggtgtcggaagcgacgccagatccgtcatcctcctttcgctctccaaagtagatacctccgacgagctctcggacaatgatgaagtcggtgccctcaacgtttcggatgggggagagatcggcgagcttgggcgacagcagctggcagggtcgcaggttggcgtacaggttcaggtcctttcgcagcttgaggagaccctgctcgggtcgcacgtcggttcgtccgtcgggagtggtccatacggtgttggcagcgcctccgacagcaccgagcataatagagtcagcctttcggcagatgtcgagagtagcgtcggtgatgggctcgccctccttctcaatggcagctcctccaatgagtcggtcctcaaacacaaactcggtgccggaggcctcagcaacagacttgagcaccttgacggcctcggcaatcacctcggggccacagaagtcgccgccgagaagaacaatcttcttggagtcagtcttggtcttcttagtttcgggttccattgtggatgtgtgtggttgtatgtgtgatgtggtgtgtggagtgaaaatctgtggctggcaaacgctcttgtatatatacgcacttttgcccgtgctatgtggaagactaaacctccgaagattgtgactcaggtagtgcggtatcggctagggacccaaaccttgtcgatgccgatagcgctatcgaacgtaccccagccggccgggagtatgtcggaggggacatacgagatcgtcaagggtttgtggccaactggtatttaaatgatgtcgacgcagtaggatgtcctgcacgggtctttttgtggggtgtggagaaaggggtgcttggagatggaagccggtagaaccgggctgcttgtgcttggagatggaagccggtagaaccgggctgcttggggggatttggggccgctgggctccaaagaggggtaggcatttcgttggggttacgtaattgcggcatttgggtcctgcgcgcatgtcccattggtcagaattagtccggataggagacttatcagccaatcacagcgccggatccacctgtaggttgggttgggtgggagcacccctccacagagtagagtcaaacagcagcagcaacatgatagttgggggtgtgcgtgttaaaggaaaaaaaagaagcttgggttatattcccgctctatttagaggttgcgggatagacgccgacggagggcaatggcgccatggaaccttgcggatatcgatacgccgcggcggactgcgtccgaaccagctccagcagcgttttttccgggccattgagccgactgcgaccccgccaacgtgtcttggcccacgcactcatgtcatgttggtgttgggaggccactttttaagtagcacaaggcacctagctcgcagcaaggtgtccgaaccaaagaagcggctgcagtggtgcaaacggggcggaaacggcgggaaaaagccacgggggcacgaattgaggcacgccctcgaatttgagacgagtcacggccccattcgcccgcgcaatggctcgccaacgcccggtcttttgcaccacatcaggttaccccaagccaaacctttgtgttaaaaagcttaacatattataccgaacgtaggtttgggcgggcttgctccgtctgtccaaggcaacatttatataagggtctgcatcgccggctcaattgaatcttttttcttcttctcttctctatattcattcttgaattaaacacacatcaaccatggagtccattgctcccttcctgccctccaagatgcctcaggacctgttcatggacctcgccagcgctatcggtgtccgagctgctccctacgtcgatcccctggaggctgccctggttgcccaggccgagaagtacattcccaccattgtccatcacactcgaggcttcctggttgccgtggagtctcccctggctcgagagctgcctctgatgaaccccttccacgtgctcctgatcgtgctcgcctacctggtcaccgtgtttgtgggtatgcagatcatgaagaactttgaacgattcgaggtcaagaccttctccctcctgcacaacttctgtctggtctccatctccgcctacatgtgcggtggcatcctgtacgaggcttatcaggccaactatggactgtttgagaacgctgccgatcacaccttcaagggtctccctatggctaagatgatctggctcttctacttctccaagatcatggagtttgtcgacaccatgatcatggtcctcaagaagaacaaccgacagatttcctttctgcacgtgtaccaccactcttccatcttcaccatctggtggctggtcaccttcgttgctcccaacggtgaagcctacttctctgctgccctgaactccttcatccacgtcatcatgtacggctactactttctgtctgccctgggcttcaagcaggtgtcgttcatcaagttctacatcactcgatcccagatgacccagttctgcatgatgtctgtccagtcttcctgggacatgtacgccatgaaggtccttggccgacctggataccccttcttcatcaccgctctgctctggttctacatgtggaccatgctcggtctcttctacaacttttaccgaaagaacgccaagctcgccaagcaggccaaggctgacgctgccaaggagaaggccagaaagctccagtaagcggccgccaccgcggcccgagattccggcctcttcggccgccaagcgacccgggtggacgtctagaggtacctagcaattaacagatagtttgccggtgataattctcttaacctcccacactcctttgacataacgatttatgtaacgaaactgaaatttgaccagatattgtgtccgc

SEQ ID NO: 9 - DNA, 10 bp, Yarrowia lipolytica
  misc_feature (8)..(8): n is a, c, g, or t
mammatgnhs

SEQ ID NO: 10 - DNA, 14688 bp, Artificial Sequence
  Plasmid pZKLeuN-29E3
cgattgttgtctactaactatcgtacgataacttcgtatagcatacattatacgaagttatcgcgtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaaaacccatatcgacatcattgacgacttcacctacgccggcactgtgctccccctcaaggaacttgctcttaagcacggtttcttcctgttcgaggacagaaagttcgcagatattggcaacactgtcaagcaccagtaccggtgtcaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctagtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacagtgctaatacgttgaactacttatacttatatgaggctcgaagaaagctgacttgtgtatgacttattctcaactacatccccagtcacaataccaccactgcactaccactacaccaaaaccatgatcaaaccacccatggacttcctggaggcagaagaacttgttatggaaaagctcaagagagagatcataacttcgtatagcatacattatacgaagttatcctgcaggtaaaggaattctggagtttctgagagaaaaaggcaagatacgtatgtaacaaagcgacgcatggtacaataataccggaggcatgtatcatagagagttagtggttcgatgatggcactggtgcctggtatgactttatacggctgactacatatttgtcctcagacatacaattacagtcaagcacttacccttggacatctgtaggtaccccccggccaagacgatctcagcgtgtcgtatgtcggattggcgtagctccctcgctcgtcaattggctcccatctactttcttctgcttggctacacccagcatgtctgctatggctcgttttcgtgccttatctatcctcccagtattaccaactctaaatgacatgatgtgattgggtctacactttcatatcagagataaggagtagcacagttgcataaaaagcccaactctaatcagcttcttcctttcttgtaattagtacaaaggtgattagcgaaatctggaagcttagttggccctaaaaaaatcaaaaaaagcaaaaaacgaaaaacgaaaaaccacagttttgagaacagggaggtaacgaaggatcgtatatatatatatatatatatatacccacggatcccgagaccggcctttgattcttccctacaaccaaccattctcaccaccctaattcacaaccatggagtctggacccatgcctgctggcattcccttccctgagtactatgacttctttatggactggaagactcccctggccatcgctgccacctacactgctgccgtcggtctcttcaaccccaaggttggcaaggtctcccgagtggttgccaagtcggctaacgcaaagcctgccgagcgaacccagtccggagctgccatgactgccttcgtctttgtgcacaacctcattctgtgtgtctactctggcatcaccttctactacatgtttcctgctatggtcaagaacttccgaacccacacactgcacgaagcctactgcgacacggatcagtccctctggaacaacgcacttggctactggggttacctcttctacctgtccaagttctacgaggtcattgacaccatcatcatcatcctgaagggacgacggtcctcgctgcttcagacctaccaccatgctggagccatgattaccatgtggtctggcatcaactaccaagccactcccatttggatctttgtggtcttcaactccttcattcacaccatcatgtactgttactatgccttcacctctatcggattccatcctcctggcaaaaagtacctgacttcgatgcagattactcagtttctggtcggtatcaccattgccgtgtcctacctcttcgttcctggctgcatccgaacacccggtgctcagatggctgtctggatcaacgtcggctacctgtttcccttgacctatctgttcgtggactttgccaagcgaacctactccaagcgatctgccattgccgctcagaaaaaggctcagtaagcggccgcattgatgattggaaacacacacatgggttatatctaggtgagagttagttggacagttatatattaaatcagctatgccaacggtaacttcattcatgtcaacgaggaaccagtgactgcaagtaatatagaatttgaccaccttgccattctcttgcactcctttactatatctcatttatttcttatatacaaatcacttcttcttcccagcatcgagctcggaaacctcatgagcaataacatcgtggatctcgtcaatagagggctttttggactccttgctgttggccaccttgtccttgctgtctggctcattctgtttcaacgccttttaattaacggagtaggtctcggtgtcggaagcgacgccagatccgtcatcctcctttcgctctccaaagtagatacctccgacgagctctcggacaatgatgaagtcggtgccctcaacgtttcggatgggggagagatcggcgagcttgggcgacagcagctggcagggtcgcaggttggcgtacaggttcaggtcctttcgcagcttgaggagaccctgctcgggtcgcacgtcggttcgtccgtcgggagtggtccatacggtgttggcagcgcctccgacagcaccgagcataatagagtcagcctttcggcagatgtcgagagtagcgtcggtgatgggctcgccctccttctcaatggcagctcctccaatgagtcggtcctcaaacacaaactcggtgccggaggcctcagcaacagacttgagcaccttgacggcctcggcaatcacctcggggccacagaagtcgccgccgagaagaacaatcttcttggagtcagtcttggtcttcttagtttcgggttccattgtggatgtgtgtggttgtatgtgtgatgtggtgtgtggagtgaaaatctgtggctggcaaacgctcttgtatatatacgcacttttgcccgtgctatgtggaagactaaacctccgaagattgtgactcaggtagtgcggtatcggctagggacccaaaccttgtcgatgccgatagcatgcgacgtcgggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgcgcccactgagctcgtctaacggacttgatatacaaccaattaaaacaaatgaaaagaaatacagttctttgtatcatttgtaacaattaccctgtacaaactaaggtattgaaatcccacaatattcccaaagtccacccctttccaaattgtcatgcctacaactcatataccaagcactaacctaccaaacaccactaaaaccccacaaaatatatcttaccgaatatacagtaacaagctaccaccacactcgttgggtgcagtcgccagcttaaagatatctatccacatcagccacaactcccttcctttaataaaccgactacacccttggctattgaggttatgagtgaatatactgtagacaagacactttcaagaagactgtttccaaaacgtaccactgtcctccactacaaacacacccaatctgcttcttctagtcaaggttgctacaccggtaaattataaatcatcatttcattagcagggcagggccctttttatagagtcttatacactagcggaccctgccggtagaccaacccgcaggcgcgtcagtttgctccttccatcaatgcgtcgtagaaacgacttactccttcttgagcagctccttgaccttgttggcaacaagtctccgacctcggaggtggaggaagagcctccgatatcggcggtagtgataccagcctcgacggactccttgacggcagcctcaacagcgtcaccggcgggcttcatgttaagagagaacttgagcatcatggcggcagacagaatggtggcgtacgcaactaacatgaatgaatacgatatacatcaaagactatgatacgcagtattgcacactgtacgagtaagagcactagccactgcactcaagtgaaaccgttgcccgggtacgagtatgagtatgtacagtatgtttagtattgtacttggacagtgcttgtatcgtacattctcaagtgtcaaacataaatatccgttgctatatcctcgcaccaccacgtagctcgctatatccctgtgttgaatccatccatcttggattgccaattgtgcacacagaaccgggcactcacttccccatccacacttgcggccgcttaagcaacgggcttgataacagcggggggggtgcccacgttgttgcggttgcggaagaacagaacacccttaccagcaccctcggcaccagcgctgggctcaacccactggcacatacgcgcactgcggtacatggcgcggatgaagccacgaggaccatcctggacatcagcccggtagtgcttgcccatgatgggcttaatggcctcggtggcctcgtccgcgttgtagaaggggatgctgctgacgtagtggtggaggacatgagtctcgatgatgccgtggagaaggtggcggccgatgaagcccatctcacggtcaatggtagcagcggcaccacggacgaagttccactcgtcgttggtgtagtggggaagggtagggtcggtgtgctggaggaaggtgatggcaacgagccagtggttaacccagaggtagggaacaaagtaccagatggccatgttgtagaaaccgaacttctgaacgaggaagtacagagcagtggccatcagaccgataccaatatcgctgaggacgatgagcttagcgtcactgttctcgtacagagggctgcggggatcgaagtggttaacaccaccgccgaggccgttatgcttgcccttgccgcgaccctcacgctggcgctcgtggtagttgtggccggtaacattggtgatgaggtagttgggccagccaacgagctgctgaaggacgagcatgagaagagtgaaagcgggggtctcctcagtaagatgagcgagctcgtgggtcatctttccgagacgagtagcctgctgctcgcgggttcggggaacgaagaccatgtcacgctccatgttgccagtggccttgtggtgctttcggtgggagatttgccagctgaagtaggggacaaggagggaagagtgaagaacccagccagtaatgtcgttgatgatgcgagaatcggagaaagcaccgtgaccgcactcatgggcaataacccagagaccagtaccgaaaagaccctgaagaacggtgtacacggcccacagaccagcgcgggcgggggtggaggggatatattcgggggtcacaaagttgtaccagatgctgaaagtggtagtcaggaggacaatgtcgcggaggatataaccgtatcccttgagagcggagcgcttgaagcagtgcttagggatggcattgtagatgtccttgatggtaaagtcgggaacctcgaactggttgccgtaggtgtcgagcatgacaccatactcggacttgggcttggcgatatcaacctcggacatggacgagagcgatgtggaagaggccgagtggcggggagagtctgaaggagagacggcggcagactcagaatccgtcacagtagttgaggtgacggtgcgtctaagcgcagggttctgcttgggcagagccgaagtggacgccatggttgatgtgtgtttaattcaagaatgaatatagagaagagaagaagaaaaaagattcaattgagccggcgatgcagacccttatataaatgttgccttggacagacggagcaagcccgcccaaacctacgttcggtataatatgttaagctttttaacacaaaggtttggcttggggtaacctgatgtggtgcaaaagaccgggcgttggcgagccattgcgcgggcgaatggggccgtgactcgtctcaaattcgagggcgtgcctcaattcgtgcccccgtggctttttcccgccgtttccgccccgtttgcaccactgcagccgcttctttggttcggacaccttgctgcgagctaggtgccttgtgctacttaaaaagtggcctcccaacaccaacatgacatgagtgcgtgggccaagacacgttggcggggtcgcagtcggctcaatggcccggaaaaaacgctgctggagctggttcggacgcagtccgccgcggcgtatggatatccgcaaggttccatagcgccattgccctccgtcggcgtctatcccgcaacctctaaatagagcgggaatataacccaagcttcttttttttcctttaacacgcacacccccaactatcatgttgctgctgctgtttgactctactctgtggaggggtgctcccacccaacccaacctacaggtggatccggcgctgtgattggctgataagtctcctatccggactaattctgaccaatgggacatgcgcgcaggacccaaatgccgcaattacgtaaccccaacgaaatgcctacccctctttggagcccagcggccccaaatccccccaagcagcccggttctaccggcttccatctccaagcacaagcagcccggttctaccggcttccatctccaagcacccctttctccacaccccacaaaaagacccgtgcaggacatcctactgcgtcgacatcatttaaattccttcacttcaagttcattcttcatctgcttctgttttactttgacaggcaaatgaagacatggtacgacttgatggaggccaagaacgccatttcaccccgagacaccgaagtgcctgaaatcctggctgcccccattgataacatcggaaactacggtattccggaaagtgtatatagaacctttccccagcttgtgtctgtggatatggatggtgtaatcccctttgagtactcgtcttggcttctctccgagcagtatgaggctctctaatctagcgcatttaatatctcaatgtatttatatatttatcttctcatgcggccgctcactgaatctttttggctcccttgtgcttcctgacgatatacgtttgcacatagaaattcaagaacaaacacaagactgtgccaacataaaagtaattgaagaaccagccaaacatcctcatcccatcttggcgataacagggaatgttcctgtacttccagacaatgtagaaaccaacattgaattgaatgatctgcattgatgtaatcagggattttggcatggggaacttcagcttgatcaatctggtccaataataaccgtacatgatccagtggatgaaaccattcaacagcacaaaaatccaaacagcttcatttcggtaattatagaacagccacatatccatcggtgcccccaaatgatggaagaattgcaaccaggtcagaggcttgcccatcagtggcaaatagaaggagtcaatatactccaggaacttgctcaaatagaacaactgcgtggtgatcctgaagacgttgttgtcaaaagccttctcgcagttgtcagacataacaccgatggtgtacatggcatatgccattgagaggaatgatcccaacgaataaatggacatgagaaggttgtaattggtgaaaacaaacttcatacgagactgaccttttggaccaagggggccaagagtgaacttcaagatgacaaatgcgatggacaagtaaagcacctcacagtgactggcatcactccagagttgggcataatcaactggttgggtaaaacttcctgcccaattgagactatttcattcaccacctccatggccattgctgtagatatgtcttgtgtgtaagggggttggggtggttgtttgtgttcttgacttttgtgttagcaagggaagacgggcaaaaaagtgagtgtggttgggagggagagacgagccttatatataatgcttgtttgtgtttgtgcaagtggacgccgaaacgggcaggagccaaactaaacaaggcagacaatgcgagcttaattggattgcctgatgggcaggggttagggctcgatcaatgggggtgcgaagtgacaaaattgggaattaggttcgcaagcaaggctgacaagactttggcccaaacatttgtacgcggtggacaacaggagccacccatcgtctgtcacgggctagccggtcgtgcgtcctgtcaggctccacctaggctccatgccactccatacaatcccactagtgtaccgctaggccgcttttagctcccatctaagacccccccaaaacctccactgtacagtgcactgtactgtgtggcgatcaagggcaagggaaaaaaggcgcaaacatgcacgcatggaatgacgtaggtaaggcgttactagactgaaaagtggcacatttcggcgtgccaaagggtcctaggtgcgtttcgcgagctgggcgccaggccaagccgctccaaaacgcctctccgactccctccagcggcctccatatccccatccctctccacagcaatgttgttaagccttgcaaacgaaaaaatagaaaggctaataagcttccaatattgtggtgtacgctgcataacgcaacaatgagcgccaaacaacacacacacacagcacacagcagcattaaccacgatgaacagcatgacattacaggtgggtgtgtaatcagggccctgattgctggtggtgggagcccccatcatgggcagatctgcgtacactgtttaaacagtgtacgcagatctactatagaggaacatttaaattgccccggagaagacggccaggccgcctagatgacaaattcaacaactcacagctgactttctgccattgccactaggggggggcctttttatatggccaagccaagctctccacgtcggttgggctgcacccaacaataaatgggtagggttgcaccaacaaagggatgggatggggggtagaagatacgaggataacggggctcaatggcacaaataagaacgaatactgccattaagactcgtgatccagcgactgacaccattgcatcatctaagggcctcaaaactacctcggaactgctgcgctgatctggacaccacagaggttccgagcactttaggttgcaccaaatgtcccaccaggtgcaggcagaaaacgctggaacagcgtgtacagtttgtcttaacaaaaagtgagggcgctgaggtcgagcagggtggtgtgacttgttatagcctttagagctgcgaaagcgcgtatggatttggctcatcaggccagattgagggtctgtggacacatgtcatgttagtgtacttcaatcgccccctggatatagccccgacaataggccgtggcctcatttttttgccttccgcacatttccattgctcgatacccacaccttgcttctcctgcacttgccaaccttaatactggtttacattgaccaacatcttacaagcggggggcttgtctagggtatatataaacagtggctctcccaatcggttgccagtctcttttttcctttctttccccacagattcgaaatctaaactacacatcacagaattccgagccgtgagtatccacgacaagatcagtgtcgagacgacgcgttttgtgtaatgacacaatccgaaagtcgctagcaacacacactctctacacaaactaacccagctctggtaccatggaggtcgtgaacgaaatcgtctccattggccaggaggttcttcccaaggtcgactatgctcagctctggtctgatgcctcgcactgcgaggtgctgtacctctccatcgccttcgtcatcctgaagttcacccttggtcctctcggacccaagggtcagtctcgaatgaagtttgtgttcaccaactacaacctgctcatgtccatctactcgctgggctccttcctctctatggcctacgccatgtacaccattggtgtcatgtccgacaactgcgagaaggctttcgacaacaatgtcttccgaatcaccactcagctgttctacctcagcaagttcctcgagtacattgactccttctatctgcccctcatgggcaagcctctgacctggttgcagttctttcaccatctcggagctcctatggacatgtggctgttctacaactaccgaaacgaagccgtttggatctttgtgctgctcaacggcttcattcactggatcatgtacggctactattggacccgactgatcaagctcaagttccctatgcccaagtccctgattacttctatgcagatcattcagttcaacgttggcttctacatcgtctggaagtaccggaacattccctgctaccgacaagatggaatgagaatgtttggctggtttttcaactacttctacgttggtactgtcctgtgtctgttcctcaacttctacgtgcagacctacatcgtccgaaagcacaagggagccaaaaagattcagtgagcggccgcatgtacatacaagattatttatagaaatgaatcgcgatcgaacaaagagtacgagtgtacgagtaggggatgatgataaaagtggaagaagttccgcatctttggatttatcaacgtgtaggacgatacttcctgtaaaaatgcaatgtctttaccataggttctgctgtagatgttattaactaccattaacatgtctacttgtacagttgcagaccagttggagtatagaatggtacacttaccaaaaagtgttgatggttgtaactacgatatataaaactgttgacgggatccccgctgatatgcctaaggaacaatcaaagaggaagatattaattcagaatgctagtatacagttagggat

SEQ ID NO: 11 - DNA, 15799 bp, Artificial Sequence
  Plasmid pZKL2-5m89C
gtacgttatcatttgaacagtgaaaggctacagtaacagaagcagttgtaaacttcattccgttgattctgtactacagtaccccactacgccgcttccgctgacactgttcaacccaaaaactacatctgcgtgcgctgtgtaaggctatcatcagatacatactgtagattctgtagatgcgaacctgcttgtatcatatacatccccctccccctgacctgcacaagcaagcaatgtgacattgatattgctgcttatctagtgccgaggatgtgaaagccgagactcaaacatttcttttactctcttgttcctgaccagacctggcggagattacgccagtatgattcttgcaggtctgagacaagcctggaacagccaacatttatttttcgaagcgagaaacatgccacaccccggcacgttcagagatgcatatgatttgtttttcgagtaacagtacccccccccccccccccaatgaaaccagtattactcacaccatcctcattcaaagcgttacactgattacgcgcccatcaacgacagcatgaggggactgctgatctgatctaatcaaatgactacaaaaatcgcaataatgaagagcaaacgacaaaaaagaaacaggttaaccaatcccgcttcaatgtctcaccacaatccagcactgtttctcattacctcctccctctaatttcagagttgcatcagggtccttgatggcgcgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgctggtttcgatttgtcttagaggaacgcatatacagtaatcatagagaataaacgatattcatttattaaagtagatagttgaggtagaagttgtaaagagtgataaatagcttagataccacagacaccctcggtgacgaagtactgcagatggtttccaatcacattgacctgctggagcagagtgttaccggcagagcactgtttattgctctggccctggcacatgacaacgttggagagaggagggtggatcaggggccagtcaataaagacctcaccagagcagtgctggtaaccgtcccagaagggcacttgagggacgatatctcctcggtgggtgattcggtagagctttcggtctttggacaccttggagacatcggggttctcctggccaaagaagagtttatcgacccagttagcaaagccagcgttaccgacaatgggctgaccaagagtaacaacgaggggatcgtggccgttaaccttgaggttgattccgaacagaagggctgcagctcctccgagagagtgaccggtgacagcaatctggtagtcgggatactgctcaatcacagagtcgagcttggggccgatctgattgtaggtgttgttgtaggactggatgaagccattgtggacaagacagtcatcacaagtagcagtagaagagatgttagcagcaagatcaaagttaattaactcacctgcaggattgagactatgaatggattcccgtgcccgtattactctactaatttgatcttggaacgcgaaaatacgtttctaggactccaaagaatctcaactcttgtccttactaaatatactacccatagttgatggtttacttgaacagagaggacatgttcacttgacccaaagtttctcgcatctcttggatatttgaacaacggcgtccactgaccgtcagttatccagtcacaaaacccccacattcatacattcccatgtacgtttacaaagttctcaattccatcgtgcaaatcaaaatcacatctattcattcatcatatataaacccatcatgtctactaacactcacaactccatagaaaacatcgactcagaacacacgctccatgcggccgcttaggaatcctgagcgtccttgacacagtgaaccacaccgactttgtgcatgtacttgagggtggaaatgatgttgcccacaatggtagggtagaagacgtaccgaactccgtgtcgttcgcaacactctcggacagcttgctgcacgaagggatagtgccaagacgacattcgaggaaagaggtgatgctcgatctggaagttgagaccgccagtaaagaacatggcaatgggtccaccgtaggtggaagaggtctccacctgagctctgtaccagtcgatctgatcggcttcaacgtccttctcggagctcttgaccttgcagttcttgtcggggattcgctccgagccatcgaagttgtgagacaagatgaaaaagaaggtgaggaaggcaccggtagcagtgggcaccagaggaatggtgatgagcagggaggttccagtgagataccagggcaagaaggcggttcgaaagatgaagaaagctcgcataacgaatgcaagggttcggtaccgtcgcagaaagccgttctctcgcatggctgtgacagactcgggaatggtgtcgttgtgctgcattcggaagatgtagagagggttgtacaccagcgaaacgccgtaggctccaagcacgaggtacatgtaccaggcctggaatcggtgaaaccactttcgagcagtgttggcagcagggtagttgtggaacacaaggaatggttctgcggactcggcatccaggtcgagaccatgctgattggtgtaggtgtgatgtcgcatgatgtgagactgcagccagatccatctggacgatccaatgacgtcgatgccgtaggcaaagagagcgttgacccagggctttttgctgatggcaccatgagaggcatcgtgctgaatggacaggccgatctgcatgtgcatgaatccagtcaagagaccccacagcaccattccggtagtagcccagtgccactcgcaaaaggcggtgacagcaatgatgccaacggttcgcagccagaatccaggtgtggcataccagttccgacctttcatgacctctcgcatagttcgcttgacgtcctgtgcaaagggagagtcgtaggtgtagacaatgtccttggaggttcggtcgtgcttgcctcgcacgaactgttgaagcagcttcgagttctcgggcttgacgtaagggtgcatggagtagaacagaggagaagcatcggaggcaccagaagcgaggatcaagtcggatccgggatggaccttggcaagaccttccagatcgtagagaatgccgtcgatggcaaccaggtcgggtcgctcgagcagctgctcggtagtaagggagagagccatggttgtgaattagggtggtgagaatggttggttgtagggaagaatcaaaggccggtctcgggatccgtgggtatatatatatatatatatatatacgatccttcgttacctccctgttctcaaaactgtggtttttcgtttttcgttttttgctttttttgatttttttagggccaactaagcttccagatttcgctaatcacctttgtactaattacaagaaaggaagaagctgattagagttgggctttttatgcaactgtgctactccttatctctgatatgaaagtgtagacccaatcacatcatgtcatttagagttggtaatactgggaggatagataaggcacgaaaacgagccatagcagacatgctgggtgtagccaagcagaagaaagtagatgggagccaattgacgagcgagggagctacgccaatccgacatacgacacgctgagatcgtcttggccggggggtacctacagatgtccaagggtaagtgcttgactgtaattgtatgtctgaggacaaatatgtagtcagccgtataaagtcataccaggcaccagtgccatcatcgaaccactaactctctatgatacatgcctccggtattattgtaccatgcgtcgctttgttacatacgtatcttgcctttttctctcagaaactccagaattctctctcttgagcttttccataacaagttcttctgcctccaggaagtccatgggtggtttgatcatggttttggtgtagtggtagtgcagtggtggtattgtgactggggatgtagttgagaataagtcatacacaagtcagctttcttcgagcctcatataagtataagtagttcaacgtattagcactgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggttttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacagatactcgtcgaccttttccttgggaaccaccaccgtcagcccttctgactcacgtattgtagccaccgacacaggcaacagtccgtggatagcagaatatgtcttgtcggtccatttctcaccaactttaggcgtcaagtgaatgttgcagaagaagtatgtgccttcattgagaatcggtgttgctgatttcaataaagtcttgagatcagtttggccagtcatgttgtggggggtaattggattgagttatcgcctacagtctgtacaggtatactcgctgcccactttatactttttgattccgctgcacttgaagcaatgtcgtttaccaaaagtgagaatgctccacagaacacaccccagggtatggttgagcaaaaaataaacactccgatacggggaatcgaaccccggtctccacggttctcaagaagtattcttgatgagagcgtatcgatcgaggaagaggacaagcggctgcttcttaagtttgtgacatcagtatccaaggcaccattgcaaggattcaaggctttgaacccgtcatttgccattcgtaacgctggtagacaggttgatcggttccctacggcctccacctgtgtcaatcttctcaagctgcctgactatcaggacattgatcaacttcggaagaaacttttgtatgccattcgatcacatgctggtttcgatttgtcttagaggaacgcatatacagtaatcatagagaataaacgatattcatttattaaagtagatagttgaggtagaagttgtaaagagtgataaatagcggccgctcactgaatctttttggctcccttgtgctttcggacgatgtaggtctgcacgtagaagttgaggaacagacacaggacagtaccaacgtagaagtagttgaaaaaccagccaaacattctcattccatcttgtcggtagcagggaatgttccggtacttccagacgatgtagaagccaacgttgaactgaatgatctgcatagaagtaatcagggacttgggcatagggaacttgagcttgatcagtcgggtccaatagtagccgtacatgatccagtgaatgaagccgttgagcagcacaaagatccaaacggcttcgtttcggtagttgtagaacagccacatgtccataggagctccgagatggtgaaagaactgcaaccaggtcagaggcttgcccatgaggggcagatagaaggagtcaatgtactcgaggaacttgctgaggtagaacagctgagtggtgattcggaagacattgttgtcgaaagccttctcgcagttgtcggacatgacaccaatggtgtacatggcgtaggccatagagaggaaggagcccagcgagtagatggacatgagcaggttgtagttggtgaacacaaacttcattcgagactgacccttgggtccgagaggaccaagggtgaacttcaggatgacgaaggcgatggagaggtacagcacctcgcagtgcgaggcatcagaccagagctgagcatagtcgaccttgggaagaacctcctggccaatggagacgatttcgttcacgacctccatggttgtgaattagggtggtgagaatggttggttgtagggaagaatcaaaggccggtctcgggatccgtgggtatatatatatatatatatatatacgatccttcgttacctccctgttctcaaaactgtggtttttcgtttttcgttttttgctttttttgatttttttagggccaactaagcttccagatttcgctaatcacctttgtactaattacaagaaaggaagaagctgattagagttgggctttttatgcaactgtgctactccttatctctgatatgaaagtgtagacccaatcacatcatgtcatttagagttggtaatactgggaggatagataaggcacgaaaacgagccatagcagacatgctgggtgtagccaagcagaagaaagtagatgggagccaattgacgagcgagggagctacgccaatccgacatacgacacgctgagatcgtcttggccggggggtacctacagatgtccaagggtaagtgcttgactgtaattgtatgtctgaggacaaatatgtagtcagccgtataaagtcataccaggcaccagtgccatcatcgaaccactaactctctatgatacatgcctccggtattattgtaccatgcgtcgctttgttacatacgtatcttgcctttttctctcagaaactccagactttggctattggtcgagataagcccggaccatagtgagtctttcacactctgtttaaacaccactaaaaccccacaaaatatatcttaccgaatatacagatctactatagaggaacaattgccccggagaagacggccaggccgcctagatgacaaattcaacaactcacagctgactttctgccattgccactaggggggggcctttttatatggccaagccaagctctccacgtcggttgggctgcacccaacaataaatgggtagggttgcaccaacaaagggatgggatggggggtagaagatacgaggataacggggctcaatggcacaaataagaacgaatactgccattaagactcgtgatccagcgactgacaccattgcatcatctaagggcctcaaaactacctcggaactgctgcgctgatctggacaccacagaggttccgagcactttaggttgcaccaaatgtcccaccaggtgcaggcagaaaacgctggaacagcgtgtacagtttgtcttaacaaaaagtgagggcgctgaggtcgagcagggtggtgtgacttgttatagcctttagagctgcgaaagcgcgtatggatttggctcatcaggccagattgagggtctgtggacacatgtcatgttagtgtacttcaatcgccccctggatatagccccgacaataggccgtggcctcatttttttgccttccgcacatttccattgctcggtacccacaccttgcttctcctgcacttgccaaccttaatactggtttacattgaccaacatcttacaagcggggggcttgtctagggtatatataaacagtggctctcccaatcggttgccagtctcttttttcctttctttccccacagattcgaaatctaaactacacatcacacaatgcctgttactgacgtccttaagcgaaagtccggtgtcatcgtcggcgacgatgtccgagccgtgagtatccacgacaagatcagtgtcgagacgacgcgttttgtgtaatgacacaatccgaaagtcgctagcaacacacactctctacacaaactaacccagctctccatggtgaaggcttctcgacaggctctgcccctcgtcatcgacggaaaggtgtacgacgtctccgcttgggtgaacttccaccctggtggagctgaaatcattgagaactaccagggacgagatgctactgacgccttcatggttatgcactctcaggaagccttcgacaagctcaagcgaatgcccaagatcaaccaggcttccgagctgcctccccaggctgccgtcaacgaagctcaggaggatttccgaaagctccgagaagagctgatcgccactggcatgtttgacgcctctcccctctggtactcgtacaagatcttgaccaccctgggtcttggcgtgcttgccttcttcatgctggtccagtaccacctgtacttcattggtgctctcgtgctcggtatgcactaccagcaaatgggatggctgtctcatgacatctgccaccaccagaccttcaagaaccgaaactggaataacgtcctgggtctggtctttggcaacggactccagggcttctccgtgacctggtggaaggacagacacaacgcccatcattctgctaccaacgttcagggtcacgatcccgacattgataacctgcctctgctcgcctggtccgaggacgatgtcactcgagcttctcccatctcccgaaagctcattcagttccaacagtactatttcctggtcatctgtattctcctgcgattcatctggtgtttccagtctgtgctgaccgttcgatccctcaaggaccgagacaaccagttctaccgatctcagtacaagaaagaggccattggactcgctctgcactggactctcaagaccctgttccacctcttctttatgccctccatcctgacctcgatgctggtgttctttgtttccgagctcgtcggtggcttcggaattgccatcgtggtcttcatgaaccactaccctctggagaagatcggtgattccgtctgggacggacatggcttctctgtgggtcagatccatgagaccatgaacattcgacgaggcatcattactgactggttctttggaggcctgaactaccagatcgagcaccatctctggcccaccctgcctcgacacaacctcactgccgtttcctaccaggtggaacagctgtgccagaagcacaacctcccctaccgaaaccctctgccccatgaaggtctcgtcatcctgctccgatacctgtcccagttcgctcgaatggccgagaagcagcccggtgccaaggctcagtaagcggccgcatgagaagataaatatataaatacattgagatattaaatgcgctagattagagagcctcatactgctcggagagaagccaagacgagtactcaaaggggattacaccatccatatccacagacacaagctggggaaaggttctatatacactttccggaataccgtagtttccgatgttatcaatgggggcagccaggatttcaggcacttcggtgtctcggggtgaaatggcgttcttggcctccatcaagtcgtaccatgtcttcatttgcctgtcaaagtaaaacagaagcagatgaagaatgaacttgaagtgaaggaatttaaatgatgtcgacgcagtaggatgtcctgcacgggtctttttgtggggtgtggagaaaggggtgcttggatcgatggaagccggtagaaccgggctgcttgtgcttggagatggaagccggtagaaccgggctgcttggggggatttggggccgctgggctccaaagaggggtaggcatttcgttggggttacgtaattgcggcatttgggtcctgcgcgcatgtcccattggtcagaattagtccggataggagacttatcagccaatcacagcgccggatccacctgtaggttgggttgggtgggagcacccctccacagagtagagtcaaacagcagcagcaacatgatagttgggggtgtgcgtgttaaaggaaaaaaaagaagcttgggttatattcccgctctatttagaggttgcgggatagacgccgacggagggcaatggcgctatggaaccttgcggatatccatacgccgcggcggactgcgtccgaaccagctccagcagcgttttttccgggccattgagccgactgcgaccccgccaacgtgtcttggcccacgcactcatgtcatgttggtgttgggaggccactttttaagtagcacaaggcacctagctcgcagcaaggtgtccgaaccaaagaagcggctgcagtggtgcaaacggggcggaaacggcgggaaaaagccacgggggcacgaattgaggcacgccctcgaatttgagacgagtcacggccccattcgcccgcgcaatggctcgccaacgcccggtcttttgcaccacatcaggttaccccaagccaaacctttgtgttaaaaagcttaacatattataccgaacgtaggtttgggcgggcttgctccgtctgtccaaggcaacatttatataagggtctgcatcgccggctcaattgaatcttttttcttcttctcttctctatattcattcttgaattaaacacacatcaaccatgggcgtattcattaaacaggagcagcttccggctctcaagaagtacaagtactccgccgaggatcactcgttcatctccaacaacattctgcgccccttctggcgacagtttgtcaaaatcttccctctgtggatggcccccaacatggtgactctgctgggcttcttctttgtcattgtgaacttcatcaccatgctcattgttgatcccacccacgaccgcgagcctcccagatgggtctacctcacctacgctctgggtctgttcctttaccagacatttgatgcctgtgacggatcccatgcccgacgaactggccagagtggaccccttggagagctgtttgaccactgtgtcgacgccatgaatacctctctgattctcacggtggtggtgtccaccacccatatgggatataacatgaagctactgattgtgcagattgccgctctcggaaacttctacctgtcgacctgggagacctaccataccggaactctgtacctttctggcttctctggtcctgttgaaggtatcttgattctggtggctcttttcgtcctcaccttcttcactggtcccaacgtgtacgctctgaccgtctacgaggctcttcccgagtccatcacttcgctgctgcctgccagcttcctggacgtcaccatcacccagatctacattggattcggagtgctgggcatggtgttcaacatctacggcgcctgcggaaacgtgatcaagtactacaacaacaagggcaagagcgctctccccgccattctcggaatcgccccctttggcatcttctacgtcggcgtctttgcctgggcccatgttgctcctctgcttctctccaagtacgccatcgtctatctgtttgccattggggctgcctttgccatgcaagtcggccagatgattcttgcccatctcgtgcttgctccctttccccactggaacgtgctgctcttcttcccctttgtgggactggcagtgcactacattgcacccgtgtttggctgggacgccgatatcgtgtcggttaacactctcttcacctgttttggcgccaccctctccatttacgccttctttgtgcttgagatcatcgacgagatcaccaactacctcgatatctggtgtctgcgaatcaagtaccctcaggagaagaagaccgaataagcggccgcatggagcgtgtgttctgagtcgatgttttctatggagttgtgagtgttagtagacatgatgggtttatatatgatgaatgaatagatgtgattttgatttgcacgatggaattgagaactttgtaaacgtacatgggaatgtatgaatgtgggggttttgtgactggataactgacggtcagtggacgccgttgttcaaatatccaagagatgcgagaaactttgggtcaagtgaacatgtcctctctgttcaagtaaaccatcaactatgggtagtatatttagtaaggacaagagttgagattctttggagtcctagaaacgtattttcgcgttccaagatcaaattagtagagtaatacgggcacgggaatccattcatagtctcaatcctgcaggtgagttaattaatcgagcttggcgtaatcatggtcatagctgtttcctgtgtgaaattgttatccgctcacaattccacacaac

SEQ ID NO: 12 - DNA, 14619 bp, Artificial Sequence
  Plasmid pZP2-85m98F
cgatggaagccggtagaaccgggctgcttgtgcttggagatggaagccggtagaaccgggctgcttggggggatttggggccgctgggctccaaagaggggtaggcatttcgttggggttacgtaattgcggcatttgggtcctgcgcgcatgtcccattggtcagaattagtccggataggagacttatcagccaatcacagcgccggatccacctgtaggttgggttgggtgggagcacccctccacagagtagagtcaaacagcagcagcaacatgatagttgggggtgtgcgtgttaaaggaaaaaaaagaagcttgggttatattcccgctctatttagaggttgcgggatagacgccgacggagggcaatggcgctatggaaccttgcggatatccatacgccgcggcggactgcgtccgaaccagctccagcagcgttttttccgggccattgagccgactgcgaccccgccaacgtgtcttggcccacgcactcatgtcatgttggtgttgggaggccactttttaagtagcacaaggcacctagctcgcagcaaggtgtccgaaccaaagaagcggctgcagtggtgcaaacggggcggaaacggcgggaaaaagccacgggggcacgaattgaggcacgccctcgaatttgagacgagtcacggccccattcgcccgcgcaatggctcgccaacgcccggtcttttgcaccacatcaggttaccccaagccaaacctttgtgttaaaaagcttaacatattataccgaacgtaggtttgggcgggcttgctccgtctgtccaaggcaacatttatataagggtctgcatcgccggctcaattgaatcttttttcttcttctcttctctatattcattcttgaattaaacacacatcaaccatggtcaagcgacccgctctgcctctcaccgtggacggtgtcacctacgacgtttctgcctggctcaaccaccatcccggaggtgccgacattatcgagaactaccgaggtcgggatgctaccgacgtcttcatggttatgcactccgagaacgccgtgtccaaactcagacgaatgcccatcatggaaccttcctctcccctgactccaacacctcccaagccaaactccgacgaacctcaggaggatttccgaaagctgcgagacgagctcattgctgcaggcatgttcgatgcctctcccatgtggtacgcttacaagaccctgtcgactctcggactgggtgtccttgccgtgctgttgatgacccagtggcactggtacctggttggtgctatcgtcctcggcattcactttcaacagatgggatggctctcgcacgacatttgccatcaccagctgttcaaggaccgatccatcaacaatgccattggcctgctcttcggaaacgtgcttcagggcttttctgtcacttggtggaaggaccgacacaacgctcatcactccgccaccaacgtgcagggtcacgatcccgacatcgacaacctgcctctcctggcgtggtccaaggaggacgtcgagcgagctggcccgttttctcgacggatgatcaagtaccaacagtattacttctttttcatctgtgcccttctgcgattcatctggtgctttcagtccattcatactgccacgggtctcaaggatcgaagcaatcagtactatcgaagacagtacgagaaggagtccgtcggtctggcactccactggggtctcaaggccttgttctactatttctacatgccctcgtttctcaccggactcatggtgttctttgtctccgagctgcttggtggcttcggaattgccatcgttgtcttcatgaaccactaccctctggagaagattcaggactccgtgtgggatggtcatggcttctgtgctggacagattcacgagaccatgaacgttcagcgaggcctcgtcacagactggtttttcggtggcctcaactaccagatcgaacatcacctgtggcctactcttcccagacacaacctcaccgctgcctccatcaaagtggagcagctgtgcaagaagcacaacctgccctaccgatctcctcccatgctcgaaggtgtcggcattcttatctcctacctgggcaccttcgctcgaatggttgccaaggcagacaaggcctaagcggccgcattgatgattggaaacacacacatgggttatatctaggtgagagttagttggacagttatatattaaatcagctatgccaacggtaacttcattcatgtcaacgaggaaccagtgactgcaagtaatatagaatttgaccaccttgccattctcttgcactcctttactatatctcatttatttcttatatacaaatcacttcttcttcccagcatcgagctcggaaacctcatgagcaataacatcgtggatctcgtcaatagagggctttttggactccttgctgttggccaccttgtccttgctgtttaaacatcgtggttaatgctgctgtgtgctgtgtgtgtgtgttgtttggcgctcattgttgcgttatgcagcgtacaccacaatattggaagcttattagcctttctattttttcgtttgcaaggcttaacaacattgctgtggagagggatggggatatggaggccgctggagggagtcggagaggcgttttggagcggcttggcctggcgcccagctcgcgaaacgcacctaggaccctttggcacgccgaaatgtgccacttttcagtctagtaacgccttacctacgtcattccatgcgtgcatgtttgcgccttttttcccttgcccttgatcgccacacagtacagtgcactgtacagtggaggttttgggggggtcttagatgggagctaaaagcggcctagcggtacactagtgggattgtatggagtggcatggagcctaggtggagcctgacaggacgcacgaccggctagcccgtgacagacgatgggtggctcctgttgtccaccgcgtacaaatgtttgggccaaagtcttgtcagccttgcttgcgaacctaattcccaattttgtcacttcgcacccccattgatcgagccctaacccctgcccatcaggcaatccaattaagctcgcattgtctgccttgtttagtttggctcctgcccgtttcggcgtccacttgcacaaacacaaacaagcattatatataaggctcgtctctccctcccaaccacactcacttttttgcccgtcttcccttgctaacacaaaagtcaagaacacaaacaaccaccccaacccccttacacacaagacatatctacagcaatggccatggctctctcccttactaccgagcagctgctcgagcgacccgacctggttgccatcgacggcattctctacgatctggaaggtcttgccaaggtccatcccggatccgacttgatcctcgcttctggtgcctccgatgcttctcctctgttctactccatgcacccttacgtcaagcccgagaactcgaagctgcttcaacagttcgtgcgaggcaagcacgaccgaacctccaaggacattgtctacacctacgactctccctttgcacaggacgtcaagcgaactatgcgagaggtcatgaaaggtcggaactggtatgccacacctggattctggctgcgaaccgttggcatcattgctgtcaccgccttttgcgagtggcactgggctactaccggaatggtgctgtggggtctcttgactggattcatgcacatgcagatcggcctgtccattcagcacgatgcctctcatggtgccatcagcaaaaagccctgggtcaacgctctctttgcctacggcatcgacgtcattggatcgtccagatggatctggctgcagtctcacatcatgcgacatcacacctacaccaatcagcatggtctcgacctggatgccgagtccgcagaaccattccttgtgttccacaactaccctgctgccaacactgctcgaaagtggtttcaccgattccaggcctggtacatgtacctcgtgcttggagcctacggcgtttcgctggtgtacaaccctctctacatcttccgaatgcagcacaacgacaccattcccgagtctgtcacagccatgcgagagaacggctttctgcgacggtaccgaacccttgcattcgttatgcgagctttcttcatctttcgaaccgccttcttgccctggtatctcactggaacctccctgctcatcaccattcctctggtgcccactgctaccggtgccttcctcaccttctttttcatcttgtctcacaacttcgatggctcggagcgaatccccgacaagaactgcaaggtcaagagctccgagaaggacgttgaagccgatcagatcgactggtacagagctcaggtggagacctcttccacctacggtggacccattgccatgttctttactggcggtctcaacttccagatcgagcatcacctctttcctcgaatgtcgtcttggcactatcccttcgtgcagcaagctgtccgagagtgttgcgaacgacacggagttcggtacgtcttctaccctaccattgtgggcaacatcatttccaccctcaagtacatgcacaaagtcggtgtggttcactgtgtcaaggacgctcaggattcctaagcggccgcatgagaagataaatatataaatacattgagatattaaatgcgctagattagagagcctcatactgctcggagagaagccaagacgagtactcaaaggggattacaccatccatatccacagacacaagctggggaaaggttctatatacactttccggaataccgtagtttccgatgttatcaatgggggcagccaggatttcaggcacttcggtgtctcggggtgaaatggcgttcttggcctccatcaagtcgtaccatgtcttcatttgcctgtcaaagtaaaacagaagcagatgaagaatgaacttgaagtgaaggaatttaaatgtaacgaaactgaaatttgaccagatattgtgtccgcggtggagctccagcttttgttccctttagtgagggttaatttcgagcttggcgtaatcatggtcatagctgtttcctgtgtgaaattgttatccgctcacaagcttccacacaacgtacgggcgtcgttgcttgtgtgatttttgaggacccatccctttggtatataagtatactctggggttaaggttgcccgtgtagtctaggttatagttttcatgtgaaataccgagagccgagggagaataaacgggggtatttggacttgtttttttcgcggaaaagcgtcgaatcaaccctgcgggccttgcaccatgtccacgacgtgtttctcgccccaattcgccccttgcacgtcaaaattaggcctccatctagacccctccataacatgtgactgtggggaaaagtataagggaaaccatgcaaccatagacgacgtgaaagacggggaggaaccaatggaggccaaagaaatggggtagcaacagtccaggagacagacaaggagacaaggagagggcgcccgaaagatcggaaaaacaaacatgtccaattggggcagtgacggaaacgacacggacacttcagtacaatggaccgaccatctccaagccagggttattccggtatcaccttggccgtaacctcccgctggtacctgatattgtacacgttcacattcaatatactttcagctacaataagagaggctgtttgtcgggcatgtgtgtccgtcgtatggggtgatgtccgagggcgaaattcgctacaagcttaactctggcgcttgtccagtatgaatagacaagtcaagaccagtggtgccatgattgacagggaggtacaagacttcgatactcgagcattactcggacttgtggcgattgaacagacgggcgatcgcttctcccccgtattgccggcgcgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgcgctgatgacactttggtctgaaagagatgcattttgaatcccaaacttgcagtgcccaagtgacatacatctccgcgttttggaaaatgttcagaaacagttgattgtgttggaatggggaatggggaatggaaaaatgactcaagtatcaattccaaaaacttctctggctggcagtacctactgtccatactactgcattttctccagtcaggccactctatactcgacgacacagtagtaaaacccagataatttcgacataaacaagaaaacagacccaataatatttatatatagtcagccgtttgtccagttcagactgtaatagccgaaaaaaaatccaaagtttctattctaggaaaatatattccaatatttttaattcttaatctcatttattttattctagcgaaatacatttcagctacttgagacatgtgatacccacaaatcggattcggactcggttgttcagaagagcatatggcattcgtgctcgcttgttcacgtattcttcctgttccatctcttggccgacaatcacacaaaaatggggttttttttttaattctaatgattcattacagcaaaattgagatatagcagaccacgtattccataatcaccaaggaagttcttgggcgtcttaattaactcacctgcaggattgagactatgaatggattcccgtgcccgtattactctactaatttgatcttggaacgcgaaaatacgtttctaggactccaaagaatctcaactcttgtccttactaaatatactacccatagttgatggtttacttgaacagagaggacatgttcacttgacccaaagtttctcgcatctcttggatatttgaacaacggcgtccactgaccgtcagttatccagtcacaaaacccccacattcatacattcccatgtacgtttacaaagttctcaattccatcgtgcaaatcaaaatcacatctattcattcatcatatataaacccatcatgtctactaacactcacaactccatagaaaacatcgactcagaacacacgctccatgcggccgcttactgagccttggcaccgggctgcttctcggccattcgagcgaactgggacaggtatcggagcaggatgacgagaccttcatggggcagagggtttcggtaggggaggttgtgcttctggcacagctgttccacctggtaggaaacggcagtgaggttgtgtcgaggcagggtgggccagagatggtgctcgatctggtagttcaggcctccaaagaaccagtcagtaatgatgcctcgtcgaatgttcatggtctcatggatctgacccacagagaagccatgtccgtcccagacggaatcaccgatcttctccagagggtagtggttcatgaagaccacgatggcaattccgaagccaccgacgagctcggaaacaaagaacaccagcatcgaggtcaggatggagggcataaagaagaggtggaacagggtcttgagagtccagtgcagagcgagtccaatggcctctttcttgtactgagatcggtagaactggttgtctcggtccttgagggatcgaacggtcagcacagactggaaacaccagatgaatcgcaggagaatacagatgaccaggaaatagtactgttggaactgaatgagctttcgggagatgggagaagctcgagtgacatcgtcctcggaccaggcgagcagaggcaggttatcaatgtcgggatcgtgaccctgaacgttggtagcagaatgatgggcgttgtgtctgtccttccaccaggtcacggagaagccctggagtccgttgccaaagaccagacccaggacgttattccagtttcggttcttgaaggtctggtggtggcagatgtcatgagacagccatcccatttgctggtagtgcataccgagcacgagagcaccaatgaagtacaggtggtactggaccagcatgaagaaggcaagcacgccaagacccagggtggtcaagatcttgtacgagtaccagaggggagaggcgtcaaacatgccagtggcgatcagctcttctcggagctttcggaaatcctcctgagcttcgttgacggcagcctggggaggcagctcggaagcctggttgatcttgggcattcgcttgagcttgtcgaaggcttcctgagagtgcataaccatgaaggcgtcagtagcatctcgtccctggtagttctcaatgatttcagctccaccagggtggaagttcacccaagcggagacgtcgtacacctttccgtcgatgacgaggggcagagcctgtcgagaagccttcacggatcccatgacggccagagagtcgtagtaggtagcgggaggaagtccggcaggtcgagcgggaccggcgccctgaatctttttggctcccttgtgctttcggacgatgtaggtctgcacgtagaagttgaggaacagacacaggacagtaccaacgtagaagtagttgaaaaaccagccaaacattctcattccatcttgtcggtagcagggaatgttccggtacttccagacgatgtagaagccaacgttgaactgaatgatctgcatagaagtaatcagggacttgggcatagggaacttgagcttgatcagtcgggtccaatagtagccgtacatgatccagtgaatgaagccgttgagcagcacaaagatccaaacggcttcgtttcggtagttgtagaacagccacatgtccataggagctccgagatggtgaaagaactgcaaccaggtcagaggcttgcccatgaggggcagatagaaggagtcaatgtactcgaggaacttgctgaggtagaacagctgagtggtgattcggaagacattgttgtcgaaagccttctcgcagttgtcggacatgacaccaatggtgtacatggcgtaggccatagagaggaaggagcccagcgagtagatggacatgagcaggttgtagttggtgaacacaaacttcattcgagactgacccttgggtccgagaggaccaagggtgaacttcaggatgacgaaggcgatggagaggtacagcacctcgcagtgcgaggcatcagaccagagctgagcatagtcgaccttgggaagaacctcctggccaatggagacgatttcgttcacgacctccatggttgtgaattagggtggtgagaatggttggttgtagggaagaatcaaaggccggtctcgggatccgtgggtatatatatatatatatatatatacgatccttcgttacctccctgttctcaaaactgtggtttttcgtttttcgttttttgctttttttgatttttttagggccaactaagcttccagatttcgctaatcacctttgtactaattacaagaaaggaagaagctgattagagttgggctttttatgcaactgtgctactccttatctctgatatgaaagtgtagacccaatcacatcatgtcatttagagttggtaatactgggaggatagataaggcacgaaaacgagccatagcagacatgctgggtgtagccaagcagaagaaagtagatgggagccaattgacgagcgagggagctacgccaatccgacatacgacacgctgagatcgtcttggccggggggtacctacagatgtccaagggtaagtgcttgactgtaattgtatgtctgaggacaaatatgtagtcagccgtataaagtcataccaggcaccagtgccatcatcgaaccactaactctctatgatacatgcctccggtattattgtaccatgcgtcgctttgttacatacgtatcttgcctttttctctcagaaactccagaattctctctcttgagcttttccataacaagttcttctgcctccaggaagtccatgggtggtttgatcatggttttggtgtagtggtagtgcagtggtggtattgtgactggggatgtagttgagaataagtcatacacaagtcagctttcttcgagcctcatataagtataagtagttcaacgtattagcactgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggttttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacagatactcgtcgaccttttccttgggaaccaccaccgtcagcccttctgactcacgtattgtagccaccgacacaggcaacagtccgtggatagcagaatatgtcttgtcggtccatttctcaccaactttaggcgtcaagtgaatgttgcagaagaagtatgtgccttcattgagaatcggtgttgctgatttcaataaagtcttgagatcagtttggccagtcatgttgtggggggtaattggattgagttatcgcctacagtctgtacaggtatactcgctgcccactttatactttttgattccgctgcacttgaagcaatgtcgtttaccaaaagtgagaatgctccacagaacacaccccagggtatggttgagcaaaaaataaacactccgatacggggaatcgaaccccggtctccacggttctcaagaagtattcttgatgagagcgtat

SEQ ID NO: 13 - DNA, 15119 bp, Artificial Sequence
  Plasmid pZSCP-Ma83
gtacgacccctctcaggccaagcagaaggctgagtccatcaagaaggccaacgctatcattgtcttcaacctcaagaacaaggctggcaagaccgagtcttggtaccttgacctcaagaacgacggtgacgtcggcaagggcaacaagtcccccaagggtgatgctgacatccagctcactctctctgacgaccacttccagcagctcgttgagggtaaggctaacgcccagcgactcttcatgaccggcaagctcaaggttaagggcaacgtcatgaaggctgccgccattgagggtatcctcaagaacgctcagaacaacctctaagcgcatcatttattgattaattgatgatttactatattgatttcgcaactgtagtgtgattgtatgtgatctggctcgtaggcttcagtaaatactagacgggtatcctacgtagttgtatcatacatcgagcctgtggttacttgtacaataattcgtaatgtagagataccccttgatccattgcctgtttctaacatacaatgatctccacgcaataatcccactcttgactaaaagttgctactcttgcacggttacctcggcatagtcacgcctctcttgtctcgtctcgaacgcacaaagtcaattgacaacgccactcactcgagtgtgccccaacagggcaccatatcgactaatttgaggccaactagggtgattttggatggaatttgatcggaaaaaatagctgcagaaattcctggagagaaaaattgaccgcatccacatggtttgaccaaaaaatcgtctccatctctgtgctcaactctcctgacgagatatgcgcgcgcacccccacatgatgtgattgatctcaacaaacttcacccagacccttatctttccgggaaacttactgtataagtggtcgtgcgaacagaaagtgtgcgcactttaggtgtctagatccgattgttctcgttctgataatgagccagccccgcgaggcaatgttttttacaattgaaaacttcgttaaccactcacattaccgtttttgccccatatttaccctctggtacactccctcttgcatacacacacactgcagtgaaaatgcactccgttagcaccgttgtgattggttcagggcacgagtttggtggtttaaggcgcaactacatcaatatgaaaacaggagacgctgaaaaggggtaatatcggactgctgctatgttgtatgtactgcatgacgaattggtgttattcaagaccgtggcacaggttgctgcggtacgagacctggtagcttctctaaacggcatgtctaggtggcgcgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgcgtcactaatcaaggatacctaccatgccactatgatgtttgcaggaggtgtacctcggcagtcatcaaaaaatggaactactggctttagatcttgttgtatggcatcgcgcctaaaaaagaaacccccttccagcgagctactacaagtagttgtagttgcgggcgttggataccgaaagtcacaagcacatgtcgaagctctcatctgaaacaccgacagtcgtctgcaccccgcaagtctcggttcgtaccagcaccaatgttaggcagaactatacacaagagggcggacgatcacttcggcgttaggcaactgaaggctattttcggctggtactgtaggggacagaggaaacgcaagtgattagtaaatcggataataggcctgttagtttaccgaaatggtgggggaggggttccgtggatatcttgaagttatggaggctgatcgttatttgtggggatggatatcattgtatggacatactgtagctactgtataaacaacggatcttacacctgcctcttgtatgcccattgcttgatcatctatcgtgttactgtacatatacaatagatatagggaagaaaagccggaagtagagaccatagtctggcagaagtaacggcctcgggtcgagagaactataacaaagtccaacggcgggtcttagaatagccccaaggatcacacagttccgcaatccagtttcacatgttccgttgcatggacttttgcatgtctactgttgctacgattcccccattgcaaccacagtttggggttaccccgcattatattagcatgattacgaaagagataagtatcatatggaacatgtgaagggtagtatgcaggtccggcggagaaagagaatgacgttttcattaagcgattcgcttggcggcttgtgggggatgtgacgatacttacggtaaagaccctgtgtgagagctggtactcgctcgttacttcgctgatctgttgggccgtcaatcgaatctcgtggaacttgcattcttcttaactgtgtctatacaagacacctaatgaaacatacaagctaccgaaatcattttactcgtactgaccggtacggtacttgcacaagtagtgaaacttccgaaaatagccagcctcatgcatcatcgcttcaccccttctgttgacctcaaaagcattccaacggtaaaaaattataacgccgccaactggatggttgtgacggcgttgaccaccaatgtgtgggggctggcggtaggaccgagcttattcgtcccaataagctctttggatttgattctttggggtgtgtggtaaaattcacatggggaagaacacggtggcagtttgaggcagaggcccagcgtgtagttcctagggcatgaatataccgaactcatggcgcagaattgagctgaatgcgcaaaaagctacaggatcaaccgcgttagaaatgccgcaaatgtccactaattccccggactgttccaaatgattctgtggggataaatctcaaactgggttaggctttgtcacgtttctttgtgtcgtgtcggttcgtccggggcaatgtgcccacgcttggctgtctccctacacctcggtaaaaactatcacatgctgcccctctcgagcaagcattaaatgcatatagtcaatctaacgacatatatataggtagggtgcatcctccggtttagctccccagaatatctcttattcattacacaaaaacaacaatgtctctcaaggtcgacggcttcacttcttaattaagttgcgacacatgtcttgatagtatcttgaattctctctcttgagcttttccataacaagttcttctgcctccaggaagtccatgggtggtttgatcatggttttggtgtagtggtagtgcagtggtggtattgtgactggggatgtagttgagaataagtcatacacaagtcagctttcttcgagcctcatataagtataagtagttcaacgtattagcactgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggttttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacagatactcgtcgaccttttccttgggaaccaccaccgtcagcccttctgactcacgtattgtagccaccgacacaggcaacagtccgtggatagcagaatatgtcttgtcggtccatttctcaccaactttaggcgtcaagtgaatgttgcagaagaagtatgtgccttcattgagaatcggtgttgctgatttcaataaagtcttgagatcagtttggccagtcatgttgtggggggtaattggattgagttatcgcctacagtctgtacaggtatactcgctgcccactttatactttttgattccgctgcacttgaagcaatgtcgtttaccaaaagtgagaatgctccacagaacacaccccagggtatggttgagcaaaaaataaacactccgatacggggaatcgaaccccggtctccacggttctcaagaagtattcttgatgagagcgtatcgatggaagccggtagaaccgggctgcttgtgcttggagatggaagccggtagaaccgggctgcttggggggatttggggccgctgggctccaaagaggggtaggcatttcgttggggttacgtaattgcggcatttgggtcctgcgcgcatgtcccattggtcagaattagtccggataggagacttatcagccaatcacagcgccggatccacctgtaggttgggttgggtgggagcacccctccacagagtagagtcaaacagcagcagcaacatgatagttgggggtgtgcgtgttaaaggaaaaaaaagaagcttgggttatattcccgctctatttagaggttgcgggatagacgccgacggagggcaatggcgctatggaaccttgcggatatccatacgccgcggcggactgcgtccgaaccagctccagcagcgttttttccgggccattgagccgactgcgaccccgccaacgtgtcttggcccacgcactcatgtcatgttggtgttgggaggccactttttaagtagcacaaggcacctagctcgcagcaaggtgtccgaaccaaagaagcggctgcagtggtgcaaacggggcggaaacggcgggaaaaagccacgggggcacgaattgaggcacgccctcgaatttgagacgagtcacggccccattcgcccgcgcaatggctcgccaacgcccggtcttttgcaccacatcaggttaccccaagccaaacctttgtgttaaaaagcttaacatattataccgaacgtaggtttgggcgggcttgctccgtctgtccaaggcaacatttatataagggtctgcatcgccggctcaattgaatcttttttcttcttctcttctctatattcattcttgaattaaacacacatcaaccatggtcaagcgacccgctctgcctctcaccgtggacggtgtcacctacgacgtttctgcctggctcaaccaccatcccggaggtgccgacattatcgagaactaccgaggtcgggatgctaccgacgtcttcatggttatgcactccgagaacgccgtgtccaaactcagacgaatgcccatcatggaaccttcctctcccctgactccaacacctcccaagccaaactccgacgaacctcaggaggatttccgaaagctgcgagacgagctcattgctgcaggcatgttcgatgcctctcccatgtggtacgcttacaagaccctgtcgactctcggactgggtgtccttgccgtgctgttgatgacccagtggcactggtacctggttggtgctatcgtcctcggcattcactttcaacagatgggatggctctcgcacgacatttgccatcaccagctgttcaaggaccgatccatcaacaatgccattggcctgctcttcggaaacgtgcttcagggcttttctgtcacttggtggaaggaccgacacaacgctcatcactccgccaccaacgtgcagggtcacgatcccgacatcgacaacctgcctctcctggcgtggtccaaggaggacgtcgagcgagctggcccgttttctcgacggatgatcaagtaccaacagtattacttctttttcatctgtgcccttctgcgattcatctggtgctttcagtccattcatactgccacgggtctcaaggatcgaagcaatcagtactatcgaagacagtacgagaaggagtccgtcggtctggcactccactggggtctcaaggccttgttctactatttctacatgccctcgtttctcaccggactcatggtgttctttgtctccgagctgcttggtggcttcggaattgccatcgttgtcttcatgaaccactaccctctggagaagattcaggactccgtgtgggatggtcatggcttctgtgctggacagattcacgagaccatgaacgttcagcgaggcctcgtcacagactggtttttcggtggcctcaactaccagatcgaacatcacctgtggcctactcttcccagacacaacctcaccgctgcctccatcaaagtggagcagctgtgcaagaagcacaacctgccctaccgatctcctcccatgctcgaaggtgtcggcattcttatctcctacctgggcaccttcgctcgaatggttgccaaggcagacaaggcctaagcggccgcattgatgattggaaacacacacatgggttatatctaggtgagagttagttggacagttatatattaaatcagctatgccaacggtaacttcattcatgtcaacgaggaaccagtgactgcaagtaatatagaatttgaccaccttgccattctcttgcactcctttactatatctcatttatttcttatatacaaatcacttcttcttcccagcatcgagctcggaaacctcatgagcaataacatcgtggatctcgtcaatagagggctttttggactccttgctgttggccaccttgtccttgctgtttaaacagagtgtgaaagactcactatggtccgggcttatctcgaccaatagccaaagtctggagtttctgagagaaaaaggcaagatacgtatgtaacaaagcgacgcatggtacaataataccggaggcatgtatcatagagagttagtggttcgatgatggcactggtgcctggtatgactttatacggctgactacatatttgtcctcagacatacaattacagtcaagcacttacccttggacatctgtaggtaccccccggccaagacgatctcagcgtgtcgtatgtcggattggcgtagctccctcgctcgtcaattggctcccatctactttcttctgcttggctacacccagcatgtctgctatggctcgttttcgtgccttatctatcctcccagtattaccaactctaaatgacatgatgtgattgggtctacactttcatatcagagataaggagtagcacagttgcataaaaagcccaactctaatcagcttcttcctttcttgtaattagtacaaaggtgattagcgaaatctggaagcttagttggccctaaaaaaatcaaaaaaagcaaaaaacgaaaaacgaaaaaccacagttttgagaacagggaggtaacgaaggatcgtatatatatatatatatatatatacccacggatcccgagaccggcctttgattcttccctacaaccaaccattctcaccaccctaattcacaaccatggtctccaaccacctgttcgacgccatgcgagctgccgctcccggagacgcacctttcattcgaatcgacaacgctcggacctggacttacgatgacgccattgctctttccggtcgaatcgctggagctatggacgcactcggcattcgacccggagacagagttgccgtgcaggtcgagaagtctgccgaggcgttgattctctacctggcctgtcttcgaaccggagctgtctacctgcctctcaacactgcctacaccctggccgagctcgactacttcatcggcgatgccgaaccgcgtctggtggtcgttgctcccgcagctcgaggtggcgtggagacaattgccaagcgacacggtgctatcgtcgaaaccctcgacgccgatggacgaggctccttgctggaccttgctagagatgagcctgccgactttgtcgatgcttcgcgatctgccgacgatctggctgctattctctacacttccggtacaaccggacgatcgaagggtgccatgcttactcatggcaatctgctctccaacgctctcaccttgcgagactattggagagttaccgcagacgatcgactcatccatgccttgccaatctttcacactcatggtctgttcgttgctacgaacgtcacactgcttgcaggagcctcgatgtttctgctctccaagttcgatgccgacgaggtcgtttctctcatgccacaggccaccatgcttatgggcgtgcccacattctacgttcgattgctgcagagtcctcgactcgagaagggtgctgtggccagcatcagactgttcatttctggatcagctcccttgcttgccgaaacccacgccgagtttcatgctcgtactggtcacgccattctcgagcgatacggcatgacggaaaccaacatgaatacttccaacccctacgagggcaagcgtattgccggaaccgttggttttcctctgcccgacgtcactgtgcgagtcaccgatcccgccaccggtctcgttcttccacctgaagagactggcatgatcgagatcaagggacccaacgtcttcaagggctattggcgaatgcccgaaaagaccgctgccgagtttaccgcagacggtttctttatctctggagatctcggcaagatcgaccgagaaggttacgttcacattgtgggacgaggcaaggacctggtcatttccggtggctacaacatctatcccaaagaggtcgaaggcgagatcgaccagatcgagggtgtggtcgagtctgctgtcattggtgttcctcatcccgatttcggagaaggtgtcaccgctgttgtcgtgtgcaaacctggtgccgttctcgacgaaaagaccatcgtgtctgctctgcaggaccgtcttgcccgatacaagcaacccaagcggattatctttgccgacgatctgcctcgaaacactatgggaaaggttcagaagaacattcttcgacagcaatacgccgatctctacaccagacgataagcggccgcatgagaagataaatatataaatacattgagatattaaatgcgctagattagagagcctcatactgctcggagagaagccaagacgagtactcaaaggggattacaccatccatatccacagacacaagctggggaaaggttctatatacactttccggaataccgtagtttccgatgttatcaatgggggcagccaggatttcaggcacttcggtgtctcggggtgaaatggcgttcttggcctccatcaagtcgtaccatgtcttcatttgcctgtcaaagtaaaacagaagcagatgaagaatgaacttgaagtgaaggaatttaaatgatgtcgacgcagtaggatgtcctgcacgggtctttttgtggggtgtggagaaaggggtgcttggagatggaagccggtagaaccgggctgcttgtgcttggagatggaagccggtagaaccgggctgcttggggggatttggggccgctgggctccaaagaggggtaggcatttcgttggggttacgtaattgcggcatttgggtcctgcgcgcatgtcccattggtcagaattagtccggataggagacttatcagccaatcacagcgccggatccacctgtaggttgggttgggtgggagcacccctccacagagtagagtcaaacagcagcagcaacatgatagttgggggtgtgcgtgttaaaggaaaaaaaagaagcttgggttatattcccgctctatttagaggttgcgggatagacgccgacggagggcaatggcgctatggaaccttgcggatatccatacgccgcggcggactgcgtccgaaccagctccagcagcgttttttccgggccattgagccgactgcgaccccgccaacgtgtcttggcccacgcactcatgtcatgttggtgttgggaggccactttttaagtagcacaaggcacctagctcgcagcaaggtgtccgaaccaaagaagcggctgcagtggtgcaaacggggcggaaacggcgggaaaaagccacgggggcacgaattgaggcacgccctcgaatttgagacgagtcacggccccattcgcccgcgcaatggctcgccaacgcccggtcttttgcaccacatcaggttaccccaagccaaacctttgtgttaaaaagcttaacatattataccgaacgtaggtttgggcgggcttgctccgtctgtccaaggcaacatttatataagggtctgcatcgccggctcaattgaatcttttttcttcttctcttctctatattcattcttgaattaaacacacatcaaccatggagtctggacccatgcctgctggcattcccttccctgagtactatgacttctttatggactggaagactcccctggccatcgctgccacctacactgctgccgtcggtctcttcaaccccaaggttggcaaggtctcccgagtggttgccaagtcggctaacgcaaagcctgccgagcgaacccagtccggagctgccatgactgccttcgtctttgtgcacaacctcattctgtgtgtctactctggcatcaccttctactacatgtttcctgctatggtcaagaacttccgaacccacacactgcacgaagcctactgcgacacggatcagtccctctggaacaacgcacttggctactggggttacctcttctacctgtccaagttctacgaggtcattgacaccatcatcatcatcctgaagggacgacggtcctcgctgcttcagacctaccaccatgctggagccatgattaccatgtggtctggcatcaactaccaagccactcccatttggatctttgtggtcttcaactccttcattcacaccatcatgtactgttactatgccttcacctctatcggattccatcctcctggcaaaaagtacctgacttcgatgcagattactcagtttctggtcggtatcaccattgccgtgtcctacctcttcgttcctggctgcatccgaacacccggtgctcagatggctgtctggatcaacgtcggctacctgtttcccttgacctatctgttcgtggactttgccaagcgaacctactccaagcgatctgccattgccgctcagaaaaaggctcagtaagcggccgcaagtgtggatggggaagtgagtgcccggttctgtgtgcacaattggcaatccaagatggatggattcaacacagggatatagcgagctacgtggtggtgcgaggatatagcaacggatatttatgtttgacacttgagaatgtacgatacaagcactgtccaagtacaatactaaacatactgtacatactcatactcgtacccgggcaacggtttcacttgagtgcagtggctagtgctcttactcgtacagtgtgcaatactgcgtatcatagtctttgatgtatatcgtattcattcatgttagttgc

SEQ ID NO: 14 - DNA, 968 bp, Yarrowia lipolytica
  misc_feature (441)..(441): n is a, c, g, or t
gacgcagtaggatgtcctgcacgggtctttttgtggggtgtggagaaaggggtgcttggagatggaagccggtagaaccgggctgcttgtgcttggagatggaagccggtagaaccgggctgcttggggggatttggggccgctgggctccaaagaggggtaggcatttcgttggggttacgtaattgcggcatttgggtcctgcgcgcatgtcccattggtcagaattagtccggataggagacttatcagccaatcacagcgccggatccacctgtaggttgggttgggtgggagcacccctccacagagtagagtcaaacagcagcagcaacatgatagttgggggtgtgcgtgttaaaggaaaaaaaagaagcttgggttatattcccgctctatttagaggttgcgggatagacgccgacggagggcaatggcgcnatggaaccttgcggatatcnatacgccgcggcggactgcgtccgaaccagctccagcagcgttttttccgggccattgagccgactgcgaccccgccaacgtgtcttggcccacgcactcatgtcatgttggtgttgggaggccactttttaagtagcacaaggcacctagctcgcagcaaggtgtccgaaccaaagaagcggctgcagtggtgcaaacggggcggaaacggcgggaaaaagccacgggggcacgaattgaggcacgccctcgaatttgagacgagtcacggccccattcgcccgcgcaatggctcgccaacgcccggtcttttgcaccacatcaggttaccccaagccaaacctttgtgttaaaaagcttaacatattataccgaacgtaggtttgggcgggcttgctccgtctgtccaaggcaacatttatataagggtctgcatcgccggctcaattgaatcttttttcttcttctcttctctatattcattcttgaattaaacacacatcaac

SEQ ID NO: 15 - DNA, 1068 bp, Yarrowia lipolytica
  misc_feature (541)..(541): n is a, c, g, or t
tcgtctcggtacatttggttacattttgcgacaggttgaaatgaatcggccgacgctcggtagtcggaaagagccgggaccggccggcgagcataaaccggacgcagtaggatgtcctgcacgggtctttttgtggggtgtggagaaaggggtgcttggagatggaagccggtagaaccgggctgcttgtgcttggagatggaagccggtagaaccgggctgcttggggggatttggggccgctgggctccaaagaggggtaggcatttcgttggggttacgtaattgcggcatttgggtcctgcgcgcatgtcccattggtcagaattagtccggataggagacttatcagccaatcacagcgccggatccacctgtaggttgggttgggtgggagcacccctccacagagtagagtcaaacagcagcagcaacatgatagttgggggtgtgcgtgttaaaggaaaaaaaagaagcttgggttatattcccgctctatttagaggttgcgggatagacgccgacggagggcaatggcgcnatggaaccttgcggatatcnatacgccgcggcggactgcgtccgaaccagctccagcagcgttttttccgggccattgagccgactgcgaccccgccaacgtgtcttggcccacgcactcatgtcatgttggtgttgggaggccactttttaagtagcacaaggcacctagctcgcagcaaggtgtccgaaccaaagaagcggctgcagtggtgcaaacggggcggaaacggcgggaaaaagccacgggggcacgaattgaggcacgccctcgaatttgagacgagtcacggccccattcgcccgcgcaatggctcgccaacgcccggtcttttgcaccacatcaggttaccccaagccaaacctttgtgttaaaaagcttaacatattataccgaacgtaggtttgggcgggcttgctccgtctgtccaaggcaacatttatataagggtctgcatcgccggctcaattgaatcttttttcttcttctcttctctatattcattcttgaattaaacacacatcaac

SEQ ID NO: 16 - DNA, 87 bp, Yarrowia lipolytica
tatataagggtctgcatcgccggctcaattgaatcttttttcttcttctcttctctatattcattcttgaattaaacacacatcaac

(19) United States
(12) Patent Application Publication
(10) Pub. No.: US 2011/0059496 A1
(43) Pub. Date: Mar. 10, 2011

(54) GLYCERALDEHYDE-3-PHOSPHATE DEHYDROGENASE AND PHOSPHOGLYCERATE MUTASE PROMOTERS FOR GENE EXPRESSION IN OLEAGINOUS YEAST

(75) Inventor: QUINN QUN ZHU, WEST CHESTER, PA (US)

(73) Assignee: E. I. DU PONT DE NEMOURS AND COMPANY, WILMINGTON, DE (US)

(21) Appl. No.: 12/948,330

(22) Filed: Nov. 17, 2010

Related U.S. Application Data
(63) Continuation-in-part of application No. 11/773,453, filed on Jul. 31, 2007.
Application No. 11/773,453 is a division of application No. 10/869,630, filed on Jun. 16, 2004, now Pat. No. 7,259,255.
(60) Provisional application No. 60/482,263, filed on Jun. 25, 2003.

(51) Int. Cl. C12P 7/64; C12N 1/19; C07H 21/04

(52) U.S. Cl. 435/134; 435/254.2; 536/24.1

(57) ABSTRACT

Promoter regions associated with the Yarrowia lipolytica glyceraldehyde-3-phosphate dehydrogenase (gpd) gene have been found to be particularly effective for the expression of heterologous genes in yeast. Promoter regions of a Yarrowia gpd gene shown to drive high-level expression of genes involved in the production of omega-3 and omega-6 fatty acids are disclosed.

This application is a continuation-in-part of U.S. patent application Ser. No. 11/773,453, filed Jul. 31, 2007, which is a divisional of U.S. patent application Ser. No. 10/869,630, filed Jun. 16, 2004 and now granted as U.S. Pat. No. 7,259,255, which claims the benefit of U.S. Provisional Application 60/482,263, filed Jun. 25, 2003, now expired. U.S. patent application Ser. No. 11/183,664, filed Jul. 18, 2005 and now granted as U.S. Pat. No. 7,459,546, is also a continuation-in-part of U.S. patent application Ser. No. 10/869,630, supra, which claims the benefit of U.S. Provisional Application 60/482,263, supra.

### FIELD OF THE INVENTION

This invention is in the field of biotechnology. More specifically, this invention pertains to glyceraldehyde-3-phosphate dehydrogenase [“GPD”] promoter regions derived from Yarrowia lipolytica that are useful for gene expression in yeast.

### BACKGROUND OF THE INVENTION

Oleaginous yeast are defined as those organisms that are naturally capable of oil synthesis and accumulation, wherein oil accumulation ranges from at least about 25% up to about 80% of the cellular dry weight. The technology for growing oleaginous yeast with high oil content is well developed (for example, see EP 0 005 277B1; Ratledge, C., Prog. Ind. Microbiol., 16:119-206 (1982)). And, these organisms have been commercially used for a variety of purposes in the past.

Recently, the natural abilities of oleaginous yeast have been enhanced by advances in genetic engineering, resulting in organisms capable of producing polyunsaturated fatty acids [“PUFAs”], carotenoids, resveratrol and sterols. For example, significant efforts by Applicants' Assignee have demonstrated that Yarrowia lipolytica can be engineered for production of ω-3 and ω-6 fatty acids, by introducing and expressing genes encoding the ω-3/ω-6 biosynthetic pathway (U.S. Pat. No. 7,238,482; U.S. Pat. No. 7,465,564; U.S. Pat. No. 7,550,286; U.S. Pat. No. 7,588,931; U.S. Pat. Appl. Pub. No. 2006-0115881-A1; U.S. Pat. Appl. Pub. No. 2009-0093543-A1).

Recombinant production of any heterologous protein is generally accomplished by constructing an expression cassette in which the DNA coding for the protein of interest is placed under the control of a promoter suitable for the host cell. The expression cassette is then introduced into the host cell (i.e., usually by plasmid-mediated transformation or targeted integration into the host genome) and production of the heterologous protein is achieved by culturing the transformed host cell under conditions necessary for the proper function of the promoter contained within the expression cassette. Thus, the development of new host cells (e.g., transformed yeast) for recombinant production of proteins generally requires the availability of promoters that are suitable for controlling the expression of a protein of interest in the host cell.

A variety of strong promoters have been isolated from Yarrowia lipolytica that are useful for heterologous gene expression in yeast, as shown in the Table below.

TABLE 1
Characterized Yarrowia lipolytica Promoters

| Promoter | | |
|---|---|---|
| Name | Native Gene | Reference |
| XPR2 | alkaline extracellular protease | U.S. Pat. No. |
| | | 4,937,189; |
| | | EP220864 |
| TEF | translation elongation factor | U.S. Pat. No. |
| | EF1-α (tef) | 6,265,185 |
| GPD, GPM | glyceraldehyde-3-phosphate- | U.S. Pat. No. |
| | dehydrogenase (gpd), | 7,259,255 |
| | phosphoglycerate mutase (gpm) | |
| GPDIN | glyceraldehyde-3-phosphate- | U.S. Pat. No. |
| | dehydrogenase (gpd) | 7,459,546 |
| GPM/FBAIN | chimeric phosphoglycerate | U.S. Pat. No. |
| | mutase (gpm)/fructose- | 7,202,356 |
| | bisphosphate aldolase (fba1) | |
| FBA, FBAIN, | fructose-bisphosphate aldolase | U.S. Pat. No. |
| FBAINm | (fba1) | 7,202,356 |
| GPAT | glycerol-3-phosphate | U.S. Pat. No. |
| | O-acyltransferase (gpat) | 7,264,949 |
| YAT1 | ammonium transporter enzyme | U.S. Pat. Appl. |
| | (yat1) | Pub. No. |
| | | 2006-0094102-A1 |
| | | and No. |
| | | 2010-0068789-A1 |
| EXP1 | export protein | Intl. App. Pub. No. |
| | | WO 2006/052870 |

Additionally, Juretzek et al. (Biotech. Bioprocess Eng., 5:320-326 (2000)) compares the glycerol-3-phosphate dehydrogenase [“G3P”], isocitrate lyase [“ICL1”], 3-oxo-acyl-CoA thiolase [“POT1”] and acyl-CoA oxidase [“POX1”, “POX2” and “POX5”] promoters with respect to their regulation and activities during growth on different carbon sources.

Despite the utility of these known promoters, however, there is a need for new improved yeast promoters for metabolic engineering of yeast (i.e., oleaginous and non-oleaginous) and for controlling the expression of heterologous genes in yeast. Furthermore, possession of a suite of promoters that can be regulated under a variety of natural growth and induction conditions in yeast will play an important role in industrial settings, wherein economical production of heterologous and/or homologous polypeptides in commercial quantities is desirable.

It is believed that these promoter regions derived from the Yarrowia lipolytica gene encoding glyceraldehyde-3-phosphate dehydrogenase [“GPD”], will be useful in expressing heterologous and/or homologous genes in transformed yeast, including Yarrowia.

### SUMMARY OF THE INVENTION

The present invention provides methods for the expression of a coding region of interest in a transformed yeast cell, using promoters derived from upstream regions of the Yarrowia lipolytica glyceraldehyde-3-phosphate dehydrogenase (gpd) gene.

Accordingly, in a first embodiment, provided herein is a method for the expression of a coding region of interest in a transformed yeast cell comprising:
a) providing the transformed yeast cell having a chimeric gene, wherein the chimeric gene comprises:
   (1) a promoter region of a gpd Yarrowia gene; and,
   (2) the coding region of interest which is expressible in the yeast cell;
   wherein the promoter region is operably linked to the coding region of interest; and,
b) growing the transformed yeast cell of step (a) under conditions whereby the chimeric gene of step (a) is expressed.

In a second embodiment, provided herein is a method for the production of an omega-3 fatty acid or omega-6 fatty acid comprising:
a) providing a transformed oleaginous yeast comprising a chimeric gene, wherein the chimeric gene comprises:
   i) a promoter region of a gpd Yarrowia gene; and,
   ii) a coding region encoding at least one omega-3 fatty acid or omega-6 fatty acid biosynthetic pathway enzyme;
   wherein the promoter region and the coding region are operably linked; and,
b) growing the transformed oleaginous yeast of step (a) under conditions whereby the at least one omega-3 fatty acid or omega-6 fatty acid biosynthetic pathway enzyme is expressed and the omega-3 fatty acid or the omega-6 fatty acid is produced; and,
c) optionally recovering the omega-3 fatty acid or the omega-6 fatty acid.

In both methods, supra, the promoter region of a gpd Yarrowia gene comprises SEQ ID NO:16.

In some embodiments, the promoter region of a gpd Yarrowia gene may be as set forth in SEQ ID NO:15, wherein said promoter optionally comprises at least one modification selected from the group consisting of:
(a) a deletion at the 5′-terminus of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259 or 260 consecutive nucleotides, wherein the first nucleotide deleted is the thymine nucleotide [‘T’] at position 1 of SEQ ID NO:15;
(b) insertion of any two nucleotides [‘NN’] after the adenine [‘A’] nucleotide at position +160 and before the guanine [‘G’] nucleotide at position +161 of SEQ ID NO:15;
(c) insertion of a cytosine [‘C’] nucleotide at the 3′ end of SEQ ID NO:15 after the cytosine [C] nucleotide at position +1068;
(d) any combination of part (a), part (b) and part (c) above.

More preferably, the promoter region of a gpd Yarrowia gene may be as set forth in SEQ ID NO:14, wherein said promoter optionally comprises at least one modification selected from the group consisting of:
(a) a deletion at the 5′-terminus of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 consecutive nucleotides, wherein the first nucleotide deleted is the guanine nucleotide [‘G’] at position 1 of SEQ ID NO:14;
(b) insertion of a thymine nucleotide and a cytosine nucleotide [‘TC’] after the adenine [‘A’] nucleotide at position +60 and before the guanine [‘G’] nucleotide at position +61 of SEQ ID NO:14;
(c) insertion of any two nucleotides [‘NN’] after the adenine [‘A’] nucleotide at position +60 and before the guanine [‘G’] nucleotide at position +61 of SEQ ID NO:14;
(d) insertion of a cytosine [‘C’] nucleotide at the 3′ end of SEQ ID NO:14 after the cytosine [‘C’] nucleotide at position +968;
(e) any combination of part (a), part (b), part (c) and part (d) above.

The promoter region of a gpd Yarrowia gene may be selected from the group consisting of SEQ ID NO:3, SEQ ID NO:5, SEQ ID NO:6 and SEQ ID NO:7.

In various embodiments of the methods of the invention, the transformed yeast cell is an oleaginous yeast. This oleaginous yeast may be a member of a genus selected from the group consisting of Yarrowia, Candida, Rhodotorula, Rhodosporidium, Cryptococcus, Trichosporon and Lipomyces.

Additionally, provided herein is an isolated nucleic acid molecule comprising a promoter region of a gpd Yarrowia selected from the group consisting of:
(a) SEQ ID NO:3;
(b) SEQ ID NO:5;
(c) SEQ ID NO:6;
(d) SEQ ID NO:7;
(e) SEQ ID NO:14, wherein said promoter optionally comprises at least one modification selected from the group consisting of: (i) a deletion at the 5′-terminus of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 consecutive nucleotides, wherein the first nucleotide deleted is the guanine nucleotide [‘G’] at position 1 of SEQ ID NO:14; (ii) insertion of a thymine nucleotide and a cytosine nucleotide [‘TC’] after the adenine [‘A’] nucleotide at position +60 and before the guanine [‘G’] nucleotide at position +61 of SEQ ID NO:14; (iii) insertion of any two nucleotides [‘NN’] after the adenine [‘A’] nucleotide at position +60 and before the guanine [‘G’] nucleotide at position +61 of SEQ ID NO:14; (iv) insertion of a cytosine [‘C’] nucleotide at the 3′ end of SEQ ID NO:14 after the cytosine [‘C’] nucleotide at position +968; and, (v) any combination of part (i), part (ii), part (iii) and part (iv) above;
(f) SEQ ID NO:15, wherein said promoter optionally comprises at least one modification selected from the group consisting of: (i) a deletion at the 5′-terminus of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259 or 260 consecutive nucleotides, wherein the first nucleotide deleted is the thymine nucleotide [‘T’] at position 1 of SEQ ID NO:15; (ii) insertion of any two nucleotides [‘NN’] after the adenine [‘A’] nucleotide at position +160 and before the guanine [‘G’] nucleotide at position +161 of SEQ ID NO:15; (iii) insertion of a cytosine [‘C’] nucleotide at the 3′ end of SEQ ID NO:15 after the cytosine [‘C’] nucleotide at position +1068; and, (iv) any combination of part (i), part (ii) and part (iii) above; and,
(g) a promoter region comprising SEQ ID NO:16.

### Biological Deposits

The following biological material has been deposited with the American Type Culture Collection [“ATCC”], 10801 University Boulevard, Manassas, Va. 20110-2209, and bears the following designation, accession number and date of deposit.

| Biological Material | Accession No. | Date of Deposit |
|---|---|---|
| Yarrowia lipolytica Y8259 | ATCC PTA-10027 | May 14, 2009 |

The biological material listed above was deposited under the terms of the Budapest Treaty on the International Recognition of the Deposit of Microorganisms for the Purposes of Patent Procedure. The listed deposit will be maintained in the indicated international depository for at least 30 years and will be made available to the public upon the grant of a patent disclosing it. The availability of a deposit does not constitute a license to practice the subject invention in derogation of patent rights granted by government action.

### BRIEF DESCRIPTION OF THE DRAWINGS AND SEQUENCE DESCRIPTIONS

FIG. 1 graphically represents the relationship between SEQ ID NOs:1, 2, 7, 14, 15 and 16, each of which relates to glyceraldehyde-3-phosphate dehydrogenase [“GPD”] and promoter regions derived therefrom in Yarrowia lipolytica.

FIG. 2 provides plasmid maps for the following: (A) pYZGDG; and, (B) pYZDE1SB.

FIGS. 3A, 3B, 3C, 3D, 3E, 3F, 3G and 3H (which should be viewed together as FIG. 3) provide a portion of an alignment of:
(a) a 2316 bp contig comprising the 5′ non-coding and the N-terminal portion of the Yarrowia lipolytica gene encoding GPD (SEQ ID NO:1);
(b) the Y. lipolytica wildtype GPDPro promoter “GPDPro” (SEQ ID NO:2; U.S. Pat. No. 7,259,255);
(c) the Y. lipolytica composite SEQ ID NO:15 promoter;
(d) the Y. lipolytica composite SEQ ID NO:14 promoter;
(e) the Y. lipolytica modified GPD-C promoter (SEQ ID NO:3);
(f) the Y. lipolytica modified GPD-NcoI*-ClaI*-C promoter (SEQ ID NO:5);
(g) the Y. lipolytica modified GPD-TC-NcoI*-ClaI*-C promoter (SEQ ID NO:6); and,
(h) the Y. lipolytica modified GPD-NcoI*-ClaI*-C-60 promoter (SEQ ID NO:7).
   Base pair differences are highlighted with an asterisk and box. The TATA box is double-underlined.

FIG. 4 illustrates the omega-3/omega-6 fatty acid biosynthetic pathway.

FIG. 5 diagrams the development of Yarrowia lipolytica strain Y8672, producing greater than 61.8% EPA in the total lipid fraction.

FIG. 6 provides plasmid maps for the following: (A) pZKLeuN-29E3; and, (B) pZKL2-5mB89C.

FIG. 7 provides plasmid maps for the following: (A) pZP2-85m98F; and, (B) pZSCP-Ma83.

The invention can be more fully understood from the following detailed description and the accompanying sequence descriptions, which form a part of this application.

The following sequences comply with 37 C.F.R. §1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (2009) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NOs:1-16 are promoters, ORFs encoding genes or proteins (or portions thereof), or plasmids, as identified in Table 2.

TABLE 2
Summary Of Nucleic Acid SEQ ID Numbers

| | Nucleic acid |
|---|---|
| Description | SEQ ID NO. |
| Assembled contig corresponding to the | 1 |
| −1525 to +791 region of the gpd gene | (2316 bp) |
| [SEQ ID NO: 24 of U.S. Pat. No. 7,259,255] | |
| Yarrowia lipolytica putative GPD | 2 |
| promoter [“GPDPro”], corresponding to the | (971 bp) |
| −968 to +3 region of the gpd gene | |
| [SEQ ID NO: 43 of U.S. Pat. No. 7,259,255] | |
| Yarrowia lipolytica modified | 3 |
| GPD-C promoter | (969 bp) |
| Plasmid pYZGDG | 4 |
| | (9,469 bp) |
| Yarrowia lipolytica modified | 5 |
| GPD-Ncol*-Clal*-C promoter | (969 bp) |
| Yarrowia lipolytica modified | 6 |
| GPD-TC-Ncol*-Clal*-C promoter | (971 bp) |
| Yarrowia lipolytica modified | 7 |
| GPD-Ncol*-Clal*-C-60 promoter | (909 bp) |
| Plasmid pYZDE1SB | 8 |
| | (8600 bp) |
| Codon-optimized translation initiation site | 9 |
| for optimal gene expression in Yarrowia | (10 bp) |
| Plasmid pZKLeuN-29E3 | 10 |
| | (14,688 bp) |
| Plasmid pZKL2-5m89C | 11 |
| | (15,799 bp) |
| Plasmid pZP2-85m98F | 12 |
| | (14,619 bp) |
| Plasmid pZSCP-Ma83 | 13 |
| | (15,119 bp) |
| Composite SEQ ID NO: 14 GPD promoter | 14 |
| | (968 bp) |
| Composite SEQ ID NO: 15 GPD promoter | 15 |
| | (1068 bp) |
| Minimal SEQ ID NO: 16 GPD promoter | 16 |
| | (87 bp) |

### DETAILED DESCRIPTION OF THE INVENTION

All patents, patent applications, and publications cited herein are hereby incorporated by reference in their entirety.

In this disclosure, a number of terms and abbreviations are used. The following definitions are provided.

“Glyceraldehyde-3-phosphate dehydrogenase” is abbreviated GPD.

“Open reading frame” is abbreviated “ORF”.

“Polymerase chain reaction” is abbreviated “PCR”.

“American Type Culture Collection” is abbreviated “ATCC”.

“Polyunsaturated fatty acid(s)” is abbreviated “PUFA(s)”.

“Triacylglycerols” are abbreviated “TAGs”.

“Total fatty acids” are abbreviated as “TFAs”.

“Fatty acid methyl esters” are abbreviated as “FAMEs”.

As used herein the term “invention” or “present invention” is intended to refer to all aspects and embodiments of the invention as described in the claims and specification herein and should not be read so as to be limited to any particular embodiment or aspect.

The term “yeast” refers to a phylogenetically diverse grouping of single-celled fungi. Yeast do not form a specific taxonomic or phylogenetic grouping, but instead comprise a diverse assemblage of unicellular organisms that occur in the Ascomycotina and Basidiomycotina. Collectively, about 100 genera of yeast have been identified, comprising approximately 1,500 species (Kurtzman and Fell, Yeast Systematics And Phylogeny: Implications Of Molecular Identification Methods For Studies In Ecology. In C. A. Rosa and G. Peter, eds., The Yeast Handbook. Germany: Springer-Verlag Berlin Herdelberg, 2006). Yeast reproduce principally by budding (or fission) and derive energy from fermentation, via conversion of carbohydrates to ethanol and carbon dioxide. Examples of some yeast genera include, but are not limited to: Agaricostilbum, Ambrosiozyma, Arthroascus, Arxula, Ashbya, Babjevia, Bensingtonia, Botryozyma, Brettanomyces, Bullera, Candida, Clavispora, Cryptococcus, Cystofilobasidium, Debaryomyces, Dekkera, Dipodascus, Endomyces, Endomycopsella, Erythrobasidium, Fellomyces, Filobasidium, Galactomyces, Geotrichum, Guilliermondella, Hansenula, Hanseniaspora, Kazachstania, Kloeckera, Kluyveromyces, Kockovaella, Kodamaea, Komagataella, Kondoa, Lachancea, Leucosporidium, Leucosporidiella, Lipomyces, Lodderomyces, Issatchenkia, Magnusiomyces, Mastigobasidium, Metschnikowia, Monosporella, Myxozyma, Nadsonia, Nematospora, Oosporidium, Pachysolen, Pichia, Phaffia, Pseudozyma, Reniforma, Rhodosporidium, Rhodotorula, Saccharomyces, Saccharomycodes, Saccharomycopsis, Saturnispora, Schizoblastosporion, Schizosaccharomyces, Sirobasidium, Smithiozyma, Sporobolomyces, Sporopachydermia, Starmerella, Sympodiomycopsis, Sympodiomyces, Torulaspora, Tremella, Trichosporon, Trichosporiella, Trigonopsis, Udeniomyces, Wickerhamomyces, Williopsis, Xanthophyllomyces, Yarrowia, Zygosaccharomyces, Zygotorulaspora, Zymoxenogloea and Zygozyma.

The term “oleaginous” refers to those organisms that tend to store their energy source in the form of oil (Weete, In: Fungal Lipid Biochemistry, 2^{nd} Ed., Plenum, 1980). Generally, the cellular oil content of oleaginous microorganisms follows a sigmoid curve, wherein the concentration of lipid increases until it reaches a maximum at the late logarithmic or early stationary growth phase and then gradually decreases during the late stationary and death phases (Yongmanitchai and Ward, Appl. Environ. Microbiol., 57:419-25 (1991)). It is common for oleaginous microorganisms to accumulate in excess of about 25% of their dry cell weight as oil.

The term “oleaginous yeast” refers to those microorganisms classified as yeasts that can make oil. Examples of oleaginous yeast include, but are no means limited to, the following genera: Yarrowia, Candida, Rhodotorula, Rhodosporidium, Cryptococcus, Trichosporon and Lipomyces. Alternatively, organisms classified as yeasts that are engineered to make more than 25% of their dry cell weight as oil are also “oleaginous”.

The term “fermentable carbon source” will refer to a carbon source that a microorganism will metabolize to derive energy. Typical carbon sources for use in the methods herein include, but are not limited to: monosaccharides, disaccharides, oligosaccharides, polysaccharides, alkanes, fatty acids, esters of fatty acids, monoglycerides, diglycerides, triglycerides, carbon dioxide, methanol, formaldehyde, formate and carbon-containing amines. Most preferred is glucose, sucrose, invert sucrose, fructose and/or fatty acids containing between 10-22 carbons. The term “invert sucrose” (or “invert sugar”) refers to a mixture comprising equal parts of fructose and glucose resulting from the hydrolysis of sucrose. Invert sucrose may be a mixture comprising 25 to 50% glucose and 25 to 50% fructose. Invert sucrose may also comprise sucrose, the amount of which depends on the degree of hydrolysis.

The term “GPD” refers to a glyceraldehyde-3-phosphate dehydrogenase enzyme (E.C. 1.2.1.12) encoded by the gpd gene and which converts D-glyceraldehyde 3-phosphate to 3-phospho-D-glyceroyl phosphate during glycolysis.

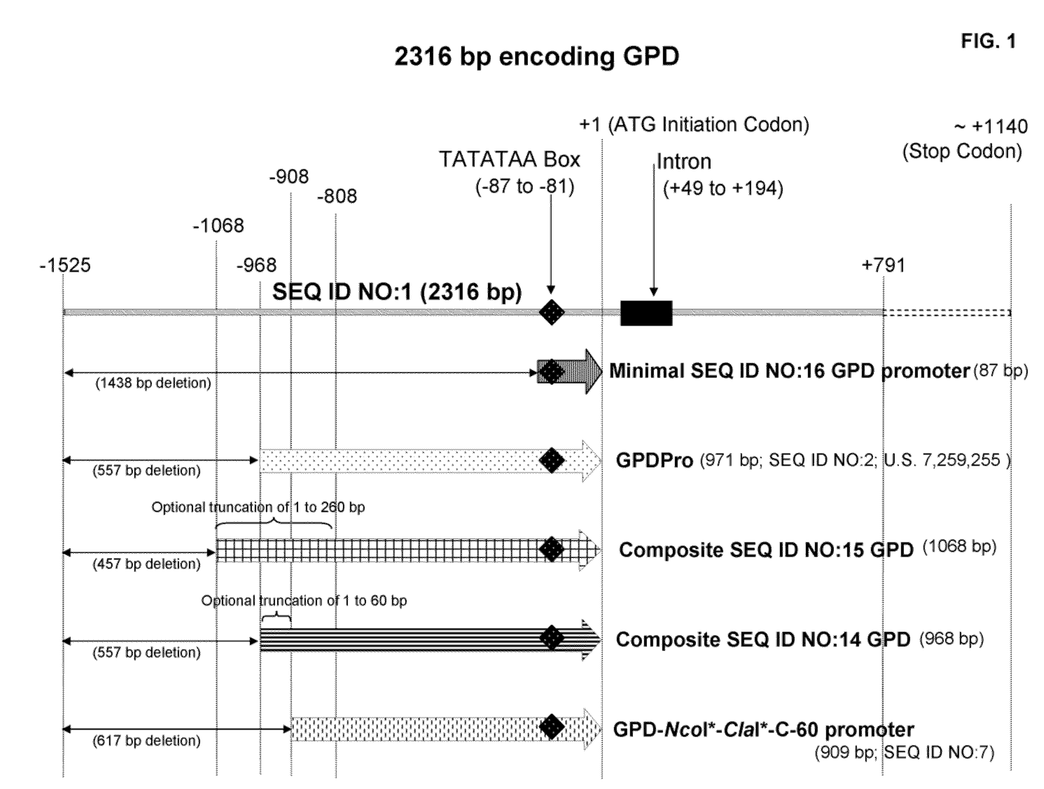

A “gpd Yarrowia gene” refers to a gene encoding GPD from a yeast of the genus Yarrowia. For example, a 2316 bp contig comprising a partial coding region of a representative gpd gene isolated from Yarrowia lipolytica is provided as SEQ ID NO:1; specifically, the sequence comprises 1525 nucleotides of 5′ upstream untranslated sequence and 791 bp of the gene (FIG. 1). Further analysis of the partial gene sequence (+1 to +791) revealed the presence of an intron (base pairs +49 to +194). Thus, the partial cDNA sequence encoding the gpd gene in Y. lipolytica is only 645 bp in length and the corresponding protein sequence is 215 amino acids (i.e., thereby lacking ˜115 amino acids that encode the C-terminus of the gene, based on alignment with other known gpd sequences).

The term “promoter region of a gpd Yarrowia gene” or “Yarrowia GPD promoter region” refers to the 5′ upstream untranslated region in front of the ‘ATG’ translation initiation codon of a Yarrowia GPD, or sequences derived therefrom, and that is necessary for expression. Thus, it is believed that such promoter regions of a gpd Yarrowia gene will comprise (at least) a “minimal promoter” region, encompassing the 5′ upstream untranslated region from the TATA box up to the ‘ATG’ translation initiation codon of a gpd Yarrowia gene. The sequence of the Yarrowia GPD promoter region may correspond exactly to native sequence upstream of the gpd Yarrowia gene (i.e., a “wildtype” or “native” Yarrowia GPD promoter); alternately, the sequence of the Yarrowia GPD promoter region may be “modified” or “mutated”, thereby comprising various substitutions, deletions, and/or insertions of one or more nucleotides relative to a wildtype or native Yarrowia GPD promoter. These modifications can result in a modified Yarrowia GPD promoter having increased, decreased or equivalent promoter activity, when compared to the promoter activity of the corresponding wildtype or native Yarrowia GPD promoter. The term “mutant promoter” or “modified promoter” will encompass natural variants and in vitro generated variants obtained using methods well known in the art (e.g., classical mutagenesis, site-directed mutagenesis and “DNA shuffling”).

U.S. Pat. No. 7,259,255 describes a wildtype Yarrowia GPD promoter region [“GPDPro”] comprising the −1525 to +3 region of SEQ ID NO:1, based on nucleotide numbering such that the ‘A’ position of the ‘ATG’ translation initiation codon is designated as +1 (i.e., SEQ ID NO:2 herein). Alternately, and yet by no means limiting in nature, a wildtype Yarrowia GPD promoter region may comprise the −1525 to −1 region of SEQ ID NO:1, the −1425 to −1 region of SEQ ID NO:1, the −1325 to −1 region of SEQ ID NO:1, the −1225 to −1 region of SEQ ID NO:1, the −1125 to −1 region of SEQ ID NO:1, the −1025 to −1 region of SEQ ID NO:1, the −968 to −1 region of SEQ ID NO:1, the −908 to −1 region of SEQ ID NO:1 or the −808 to −1 region of SEQ ID NO:1. Similarly, a modified Yarrowia GPD promoter region may comprise the promoter region of a gpd Yarrowia gene as set forth in SEQ ID NO:14, wherein said promoter optionally comprises at least one modification selected from the group consisting of:
a) a deletion at the 5′-terminus of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 consecutive nucleotides, wherein the first nucleotide deleted is the guanine nucleotide [‘G’] at position 1 of SEQ ID NO:14;
b) insertion of a thymine nucleotide and a cytosine nucleotide [‘TC’] after the adenine [‘A’] nucleotide at position +60 and before the guanine [‘G’] nucleotide at position +61 of SEQ ID NO:14;
c) insertion of any two nucleotides [‘NN’] after the adenine [‘A’] nucleotide at position +60 and before the guanine [‘G’] nucleotide at position +61 of SEQ ID NO:14;
d) insertion of a cytosine [‘C’] nucleotide at the 3′ end of SEQ ID NO:14 after the cytosine [‘C’] nucleotide at position +968; and,
e) any combination of part (a), part (b), part (c) and part (d) above.
   These examples are not intended to be limiting in nature and will be elaborated infra. FIG. 1 graphically illustrates various Yarrowia GPD promoter regions (i.e., SEQ ID NO:2 [“GPDPro”], SEQ ID NO:7 [“GPD-NcoI*-ClaI*-C-60 promoter”], SEQ ID NO:14, SEQ ID NO:15 and SEQ ID NO:16), with the 2316 bp contig comprising 1525 bp upstream of the GPD initiation codon and 791 bp of the Yarrowia gpd gene as a reference.

The term “promoter activity” will refer to an assessment of the transcriptional efficiency of a promoter. This may, for instance, be determined directly by measurement of the amount of mRNA transcription from the promoter (e.g., by Northern blotting or primer extension methods) or indirectly by measuring the amount of gene product expressed from the promoter.

The terms “polynucleotide”, “polynucleotide sequence”, “nucleic acid sequence”, “nucleic acid fragment” and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

A “substantial portion” of an amino acid or nucleotide sequence is that portion comprising enough of the amino acid sequence of a polypeptide or the nucleotide sequence of a gene to putatively identify that polypeptide or gene, either by manual evaluation of the sequence by one skilled in the art, or by computer-automated sequence comparison and identification using algorithms such as BLAST (Basic Local Alignment Search Tool; Altschul, S. F., et al., J. Mol. Biol. 215:403-410 (1993)). In general, a sequence of ten or more contiguous amino acids or thirty or more nucleotides is necessary in order to identify putatively a polypeptide or nucleic acid sequence as homologous to a known protein or gene. Moreover, with respect to nucleotide sequences, gene-specific oligonucleotide probes comprising 20-30 contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12-15 bases may be used as amplification primers in PCR in order to obtain a particular nucleic acid molecule comprising the primers. Accordingly, a “substantial portion” of a nucleotide sequence comprises enough of the sequence to specifically identify and/or isolate a nucleic acid molecule comprising the sequence.

The disclosure herein teaches partial or complete nucleotide sequences encoding one or more particular yeast promoters. The skilled artisan, having the benefit of the sequences as reported herein, may now use all or a substantial portion of the disclosed sequences for purposes known to those skilled in this art. Accordingly, the complete sequences as reported in the accompanying Sequence Listing, as well as substantial portions of those sequences as defined above, are encompassed in the present disclosure.

The term “complementary” is used to describe the relationship between nucleotide bases that are capable of hybridizing to one another. For example, with respect to DNA, adenosine is complementary to thymine and cytosine is complementary to guanine. Accordingly, isolated nucleic acid fragments that are complementary to the complete sequences as reported in the accompanying Sequence Listing, as well as those substantially similar nucleic acid sequences, are encompassed in the present disclosure.

The terms “homology”, “homologous”, “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant invention such as deletion or insertion of one or more nucleotides that do not substantially alter the functional properties of the resulting nucleic acid fragment relative to the initial, unmodified fragment. It is therefore understood, as those skilled in the art will appreciate, that the disclosure herein encompasses more than the specific exemplary sequences.

“Sequence identity” or “identity” in the context of nucleic acid or polypeptide sequences refers to the nucleic acid bases or amino acid residues in two sequences that are the same when aligned for maximum correspondence over a specified comparison window.

Thus, “percentage of sequence identity” or “percent identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity.

Methods to determine “percent identity” and “percent similarity” are codified in publicly available computer programs. Percent identity and percent similarity can be readily calculated by known methods, including but not limited to those described in: 1) Computational Molecular Biology (Lesk, A. M., Ed.) Oxford University: NY (1988); 2) Biocomputing: Informatics and Genome Projects (Smith, D. W., Ed.) Academic: NY (1993); 3) Computer Analysis of Sequence Data, Part I (Griffin, A. M., and Griffin, H. G., Eds.) Humania: NJ (1994); 4) Sequence Analysis in Molecular Biology (von Heinje, G., Ed.) Academic (1987); and, 5) Sequence Analysis Primer (Gribskov, M. and Devereux, J., Eds.) Stockton: NY (1991).

Sequence alignments and percent identity or similarity calculations may be determined using a variety of comparison methods designed to detect homologous sequences including, but not limited to, the MegAlign™ program of the LASERGENE bioinformatics computing suite (DNASTAR Inc., Madison, Wis.). Multiple alignment of the sequences is performed using the “Clustal method of alignment” which encompasses several varieties of the algorithm including the “Clustal V method of alignment” and the “Clustal W method of alignment” (described by Higgins and Sharp, CABIOS, 5:151-153 (1989); Higgins, D. G. et al., Comput. Appl. Biosci., 8:189-191(1992)) and found in the MegAlign™ (version 8.0.2) program of the LASERGENE bioinformatics computing suite (DNASTAR Inc.). After alignment of the sequences using either Clustal program, it is possible to obtain a “percent identity” by viewing the “sequence distances” table in the program.

For multiple alignments using the Clustal V method of alignment, the default values correspond to GAP PENALTY=10 and GAP LENGTH PENALTY=10. Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4.

Default parameters for multiple alignment using the Clustal W method of alignment correspond to GAP PENALTY=10, GAP LENGTH PENALTY=0.2, Delay Divergent Seqs (%)=30, DNA Transition Weight=0.5, Protein Weight Matrix=Gonnet Series, DNA Weight Matrix=IUB.

The “BLASTN method of alignment” is an algorithm provided by the National Center for Biotechnology Information [“NCBI”] to compare nucleotide sequences using default parameters, while the “BLASTP method of alignment” is an algorithm provided by the NCBI to compare protein sequences using default parameters.

It is well understood by one skilled in the art that many levels of sequence identity are useful in identifying polypeptides from other species, wherein such polypeptides have the same or similar function or activity. Likewise, suitable promoter regions (isolated polynucleotides of the present invention) are at least about 70-85% identical, and more preferably at least about 85-95% identical to the nucleotide sequences reported herein. Although preferred ranges are described above, useful examples of percent identities include any integer percentage from 70% to 100%, such as 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. Suitable Yarrowia GPD promoter regions not only have the above homologies but typically are at least 50 nucleotides in length, more preferably at least 100 nucleotides in length, more preferably at least 250 nucleotides in length, and more preferably at least 500 nucleotides in length.

“Codon degeneracy” refers to the nature in the genetic code permitting variation of the nucleotide sequence without affecting the amino acid sequence of an encoded polypeptide. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

“Synthetic genes” can be assembled from oligonucleotide building blocks that are chemically synthesized using procedures known to those skilled in the art. These oligonucleotide building blocks are annealed and then ligated to form gene segments that are then enzymatically assembled to construct the entire gene. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell, where sequence information is available. For example, the codon usage profile for Yarrowia lipolytica is provided in U.S. Pat. No. 7,125,672.

“Gene” refers to a nucleic acid fragment that expresses a specific protein, and that may refer to the coding region alone or may include regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. Chimeric genes herein will typically comprise a promoter region of a gpd Yarrowia gene operably linked to a coding region of interest. “Endogenous gene” refers to a native gene in its natural location in the genome of an organism. A “foreign” gene refers to a gene that is introduced into the host organism by gene transfer. Foreign genes can comprise native genes inserted into a non-native organism, native genes introduced into a new location within the native host, or chimeric genes. A “transgene” is a gene that has been introduced into the genome by a transformation procedure. A “codon-optimized gene” is a gene having its frequency of codon usage designed to mimic the frequency of preferred codon usage of the host cell.

“Coding sequence” refers to a DNA sequence which codes for a specific amino acid sequence. The terms “coding sequence” and “coding region” are used interchangeably herein. A “coding region of interest” is a coding region which is desired to be expressed. Such coding regions are discussed more fully hereinbelow. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to: promoters, enhancers, silencers, 5′ untranslated leader sequence (e.g., between the transcription start site and translation initiation codon), introns, polyadenylation recognition sequences, RNA processing sites, effector binding sites and stem-loop structures.

“Promoter” refers to a DNA sequence that facilitates transcription of a coding sequence, thereby enabling gene expression. In general, a promoter is typically located on the same strand and upstream of the coding sequence (i.e., 5′ of the coding sequence). Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters that cause a gene to be expressed at almost all stages of development are commonly referred to as “constitutive promoters”. It is further recognized that since in most cases the exact boundaries of regulatory sequences (especially at their 5′ end) have not been completely defined, DNA fragments of some variation may have identical promoter activity.

“Minimal promoter” refers to the minimal length of DNA sequence that is believed to be necessary to initiate basal level transcription of an operably linked coding sequence. Although promoters interact with the TATA binding protein [“TBP”] to create a transciption initiation complex from which RNA polymerase II transcribes the DNA coding sequence, only some promoters contain a TATA box to which TBP binds directly while other promoters are TATA-less promoters. For those promoters that do contain a TATA box, the minimal promoter region is herein defined as the 5′ untranslated region spanning from the TATA box to the translation initiation codon (e.g., ‘ATG’) of the coding sequence.

The “TATA box” or “Goldberg-Hogness box” is a DNA sequence (i.e., cis-regulatory element) found in the promoter region of some genes in archaea and eukaryotes. For example, approximately 24% of human genes contain a TATA box within the core promoter (Yang C, et al., Gene, 389:52-65 (2007)); phylogenetic analysis of six Saccharomyces species revealed that about 20% of the 5,700 yeast genes contained a TATA-box element (Basehoar et al., Cell, 116:699-709 (2004)). The TATA box has a core DNA sequence of 5′-TATAAA-3′ or a variant thereof and is usually located ˜200 to 25 base pairs upstream of the transcriptional start site. The transciption initiation complex forms at the site of the TATA box (Smale, and Kadonaga, T. Annual Review Of Biochemistry, 72:449-479 (2003)). This complex comprises the TATA binding protein [“TBP”], RNA polymerase II, and various transcription factors (i.e., TFIID, TFIIA, TFIIB, TFIIF, TFIIE and TFIIH). Both the TATA box itself and the distance between the TATA box and transcription start site affect activity of TATA box containing promoters in eukaryotes (Zhu et al., The Plant Cell, 7:1681-1689 (1995)).

The terms “3′ non-coding sequences”, “transcription terminator” and “termination sequences” refer to DNA sequences located downstream of a coding sequence. This includes polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The 3′ region can influence the transcription, RNA processing or stability, or translation of the associated coding sequence.

The term “enhancer” refers to a cis-regulatory sequence that can elevate levels of transcription from an adjacent eukaryotic promoter, thereby increasing transcription of the gene. Enhancers can act on promoters over many kilobases of DNA and can be 5′ or 3′ to the promoter they regulate. Enhancers can also be located within introns (Giacopelli F. et al., Gene Expr., 11:95-104 (2003)).

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA. “Messenger RNA” or “mRNA” refers to the RNA that is without introns and that can be translated into protein by the cell. “cDNA” refers to a double-stranded DNA that is complementary to, and derived from, mRNA. “Sense” RNA refers to RNA transcript that includes the mRNA and so can be translated into protein by the cell. “Antisense RNA” refers to an RNA transcript that is complementary to all or part of a target primary transcript or mRNA, and that blocks the expression of a target gene (U.S. Pat. No. 5,107,065).

The term “operably linked” refers to the association of nucleic acid sequences on a single nucleic acid molecule so that the function of one is affected by the other. For example, a promoter is operably linked with a coding sequence when it is capable of affecting the expression of that coding sequence, i.e., the coding sequence is under the transcriptional control of the promoter. Coding sequences can be operably linked to regulatory sequences in sense or antisense orientation.

The term “recombinant” refers to an artificial combination of two otherwise separated segments of sequence, e.g., by chemical synthesis or by the manipulation of isolated segments of nucleic acids by genetic engineering techniques.

The term “expression”, as used herein, refers to the transcription and stable accumulation of sense (mRNA) or antisense RNA. Expression may also refer to translation of mRNA into a protein (either precursor or mature).

“Transformation” refers to the transfer of a nucleic acid molecule into a host organism, resulting in genetically stable inheritance. The nucleic acid molecule may be a plasmid that replicates autonomously, for example, or, it may integrate into the genome of the host organism. Host organisms containing the transformed nucleic acid fragments are referred to as “transgenic” or “recombinant” or “transformed” or “transformant” organisms.

The terms “plasmid” and “vector” refer to an extra chromosomal element often carrying genes that are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing an expression cassette(s) into a cell.

The term “expression cassette” refers to a fragment of DNA containing a foreign gene and having elements in addition to the foreign gene that allow for expression of that gene in a foreign host. Generally, an expression cassette will comprise the coding sequence of a selected gene and regulatory sequences preceding (5′ non-coding sequences) and following (3′ non-coding sequences) the coding sequence that are required for expression of the selected gene product. Thus, an expression cassette is typically composed of: 1) a promoter sequence; 2) a coding sequence [“ORF”]; and, 3) a 3′ untranslated region (i.e., a terminator) that, in eukaryotes, usually contains a polyadenylation site. The expression cassette(s) is usually included within a vector, to facilitate cloning and transformation. Different expression cassettes can be transformed into different organisms including bacteria, yeast, plants and mammalian cells, as long as the correct regulatory sequences are used for each host.

The terms “recombinant construct”, “expression construct”, “chimeric construct”, “construct”, and “recombinant DNA construct” are used interchangeably herein. A recombinant construct comprises an artificial combination of nucleic acid fragments, e.g., regulatory and coding sequences that are not found together in nature. For example, a recombinant construct may comprise one or more expression cassettes. In another example, a recombinant DNA construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments described herein. The skilled artisan will also recognize that different independent transformation events will result in different levels and patterns of expression (Jones et al., EMBO J., 4:2411-2418 (1985); De Almeida et al., Mol. Gen. Genetics, 218:78-86 (1989)), and thus that multiple events must be screened in order to obtain strains displaying the desired expression level and pattern. Such screening may be accomplished by Southern analysis of DNA, Northern analysis of mRNA expression, Western and/or Elisa analyses of protein expression, formation of a specific product, phenotypic analysis or GC analysis of the PUFA products, among others.

“Introns” are sequences of non-coding DNA found in gene sequences (either in the coding region or 5′ non-coding region) in most eukaryotes. Their full function is not known; however, some enhancers are located in the introns (Giacopelli F. et al., Gene Expr., 11:95-104 (2003)). These intron sequences are transcribed, but removed from within the pre-mRNA transcript before the mRNA is translated into a protein. This process of intron removal occurs by self-splicing of the sequences (exons) on either side of the intron.

The term “altered biological activity” will refer to an activity, associated with a protein encoded by a nucleotide sequence which can be measured by an assay method, where that activity is either greater than or less than the activity associated with the native sequence. “Enhanced biological activity” refers to an altered activity that is greater than that associated with the native sequence. “Diminished biological activity” is an altered activity that is less than that associated with the native sequence.

The term “sequence analysis software” refers to any computer algorithm or software program that is useful for the analysis of nucleotide or amino acid sequences. “Sequence analysis software” may be commercially available or independently developed. Typical sequence analysis software will include, but is not limited to: 1) the GCG suite of programs (Wisconsin Package Version 9.0, Genetics Computer Group (GCG), Madison, Wis.); 2) BLASTP, BLASTN, BLASTX (Altschul et al., J. Mol. Biol., 215:403-410 (1990)); 3) DNASTAR (DNASTAR, Inc. Madison, Wis.); 4) Sequencher (Gene Codes Corporation, Ann Arbor, Mich.); and, 5) the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Plenum: New York, N.Y.). Within this description, whenever sequence analysis software is used for analysis, the analytical results are based on the “default values” of the program referenced, unless otherwise specified. As used herein “default values” will mean any set of values or parameters that originally load with the software when first initialized.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory: Cold Spring Harbor, N.Y. (1989); by Silhavy, T. J., Bennan, M. L. and Enquist, L. W., Experiments with Gene Fusions, Cold Spring Harbor Laboratory: Cold Spring Harbor, N.Y. (1984); and by Ausubel, F. M. et al., Current Protocols in Molecular Biology, published by Greene Publishing Assoc. and Wiley-Interscience, Hoboken, N.J. (1987).

A promoter useful for controlling the expression of heterologous genes in a yeast should preferably meet criteria with respect to strength, activities, pH Tolerance and inducibility, as described in U.S. Pat. No. 7,259,255. Additionally, today's complex metabolic engineering utilized for construction of yeast having the capability to produce a variety of heterologous polypeptides in commercial quantities requires a suite of promoters that are regulatable under a variety of natural growth and induction conditions.

U.S. Pat. No. 7,259,255 describes the identification of a portion of the Yarrowia lipolytica gene encoding glyceraldehyde-3-phosphate dehydrogenase [“GPD”], within a single 2316 bp contig (SEQ ID NO:1; FIG. 1). Specifically, this contig comprised 1525 bp upstream of the GPD initiation codon and 791 bp of the gpd gene, with an intron located at base pairs +49 to +194 (wherein the ‘A’ nucleotide of the ‘ATG’ translation initiation codon was designated as +1). A variety of Yarrowia GPD promoter regions were also generally described, including a putative GPD promoter region 971 nucleotides in length, designated therein as “GPDPro” (SEQ ID NO:2) and corresponding to the nucleotide region between the −968 position and the ‘ATG’ translation initiation site of the Yarrowia GPD gene (i.e., the −968 to −1 upstream region of the gpd gene and the +1 to +3 region of the gpd gene).

U.S. Pat. No. 7,259,255 also describes the creation and expression of a modified Yarrowia GPD promoter region, designated herein as “GPD-C”; however, the differences between GPDPro [SEQ ID NO:2] and GPD-C [SEQ ID NO:3] (i.e., a C insertion at +969 and deletion of the ATG at +969 to +971 of SEQ ID NO:2) were not appreciated until preparation of the present application. Upon discovery of the sequence of the GPD-C promoter (as described herein in Example 2), a variety of other modified Yarrowia GPD promoter regions were created and successfully used for expression of a variety of coding regions of interest (Examples 3 and 4).

Thus, described herein are a suite of promoter regions of a gpd Yarrowia gene, useful for driving expression of any suitable coding region of interest in a transformed yeast cell. More specifically, described herein is an isolated nucleic acid molecule comprising a promoter region of a gpd Yarrowia gene, wherein said promoter region of a gpd Yarrowia gene is set forth in SEQ ID NO:15 (corresponding to the −1068 to −1 region upstream of the Yarrowia gpd gene set forth in SEQ ID NO:1), and wherein said promoter optionally comprises at least one modification selected from the group consisting of:
(a) a deletion at the 5′-terminus of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259 or 260 consecutive nucleotides, wherein the first nucleotide deleted is the thymine nucleotide [‘T’] at position 1 of SEQ ID NO:15;
(b) insertion of any two nucleotides [‘NN’] after the adenine [‘A’] nucleotide at position +160 and before the guanine [‘G’] nucleotide at position +161 of SEQ ID NO:15;
(c) insertion of a cytosine [‘C’] nucleotide at the 3′ end of SEQ ID NO:15 after the cytosine [‘C’] nucleotide at position +1068;
(d) any combination of part (a), part (b) and part (c) above.

In more preferred embodiments, described herein is an isolated nucleic acid molecule comprising a promoter region of a gpd Yarrowia gene, wherein said promoter region of a gpd Yarrowia gene is set forth in SEQ ID NO:14 (corresponding to the −968 to −1 region upstream of the Yarrowia gpd gene set forth in SEQ ID NO:1), and wherein said promoter optionally comprises at least one modification selected from the group consisting of:
(a) a deletion at the 5′-terminus of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 consecutive nucleotides, wherein the first nucleotide deleted is the guanine nucleotide [‘G’] at position 1 of SEQ ID NO:14;
(b) insertion of a thymine nucleotide and a cytosine nucleotide [‘TC’] after the adenine [‘A’] nucleotide at position +60 and before the guanine [‘G’] nucleotide at position +61 of SEQ ID NO:14;
(c) insertion of any two nucleotides [‘NN’] after the adenine [‘A’] nucleotide at position +60 and before the guanine [‘G’] nucleotide at position +61 of SEQ ID NO:14;
(d) insertion of a cytosine [‘C’] nucleotide at the 3′ end of SEQ ID NO:14 after the cytosine [‘C’] nucleotide at position +968;
(e) any combination of part (a), part (b), part (c) and part (d) above.
   In some embodiments, the promoter region of a gpd Yarrowia gene is selected from the group consisting of SEQ ID NOs:3, 5, 6 and 7.

Although the promoter regions described above are preferred to provide relatively high levels of promoter activity, the minimal promoter region of a gpd Yarrowia gene suitable for basal level transcription initiation encompasses (at least) the 5′ upstream untranslated region from the TATA box up to the ‘ATG’ translation initiation codon of a gpd Yarrowia gene. Thus, based on the sequence set forth as SEQ ID NO:1 herein, the minimal promoter region includes the region spanning from the TATATAA sequence at −87 to −81 of SEQ ID NO:1 up to the ‘ATG’ translation initiation codon of the gpd gene, i.e., the −87 to −1 region of SEQ ID NO:1 which is set forth independently as SEQ ID NO:16.

The relationship between the promoter regions of a Yarrowia gpd gene selected from the group consisting of SEQ ID NOs:2, 3, 4, 5, 6, 7, 14, and 15, supra, is readily observed upon alignment of the individual promoter sequences. Specifically, FIG. 3 provides a portion of an alignment of:
(a) the 2316 bp contig comprising the 5′ non-coding and the N-terminal portion of the Yarrowia lipolytica gene encoding GPD (SEQ ID NO:1);
(b) the Y. lipolytica wildtype GPDPro promoter “GPDPro” (SEQ ID NO:2; U.S. Pat. No. 7,259,255);

(c) the Y. lipolytica composite SEQ ID NO:15 promoter;

(d) the Y. lipolytica composite SEQ ID NO:14 promoter;

(e) the Y. lipolytica modified GPD-C promoter (SEQ ID NO:3);

(f) the Y. lipolytica modified GPD-NcoI*-ClaI*-C promoter (SEQ ID NO:5);

(g) the Y. lipolytica modified GPD-TC-NcoI*-ClaI*-C promoter (SEQ ID NO:6); and,

(h) the Y. lipolytica modified GPD-NcoI*-ClaI*-C-60 promoter (SEQ ID NO:7).

Nucleotide differences are highlighted with a box and asterick, while the TATA box is double-underlined.

As will be obvious to one of skill the art, the above discussion is by no means limiting to the description of suitable promoter regions of a gpd Yarrowia gene. For example, alternate Yarrowia GPD promoter regions may be longer than the 1068 bp sequence of SEQ ID NO:15, thereby encompassing additional nucleotides spanning the −1525 to −1068 region of SEQ ID NO:1. Thus, for example, a suitable promoter region of a gpd Yarrowia gene could comprise the −1525 to −1 region of SEQ ID NO:1, the −1524 to −1 region, the −1523 to −1 region, the −1522 to −1 region, the −1521 to −1 region, the −1520 to −1 region, the −1519 to −1 region, the −1518 to −1 region, etc., the −1073 to −1 region, the −1072 to −1 region, the −1071 to −1 region, the −1070 to −1 region, the −1069 to −1 region, or any integer between −1525 to −1068 (thus, a suitable Yarrowia GPD promoter region could comprise nucleotides 1 to 1525 of SEQ ID NO:1, wherein the promoter region could optionally comprise a deletion at the 5′-terminus of 1 to 457 consecutive nucleotides [i.e., 1, 2, 3, 4, 5, etc. up to 457], wherein the first nucleotide deleted is the guanine nucleotide [‘G’] at position 1 of SEQ ID NO:1).

Similarly, it should be recognized that promoter fragments of various diminishing lengths may have identical promoter activity, since the exact boundaries of the regulatory sequences have not been completely defined. Thus, for example, it is also contemplated that a suitable promoter region of a gpd Yarrowia gene could also include a promoter region of SEQ ID NO:15, wherein the 5′-terminus deletion was greater than 260 consecutive nucleotides. More specifically, based on sequence analysis of the promoter region within the −1525 to +1 region of SEQ ID NO:1, and identification of a TATA box 87 bases upstream of the ATG translation initiation codon, it is hypothesized herein that the minimal promoter region that could function for basal level transcription initiation of an operably linked coding region of interest is set forth as SEQ ID NO:16. In alternate embodiments, SEQ ID NO:16 could be utilized as an enhancer to elevate levels of transcription from an adjacent eukaryotic promoter, thereby increasing transcription of a coding region of interest One of skill in the art would readily be able to conduct appropriate deletion studies to determine the appropriate length of a promoter region of a gpd Yarrowia gene required to enable the desired level of promoter activity.

More specifically, additional mutant Yarrowia GPD promoter regions may be constructed, wherein the DNA sequence of the promoter has one or more nucleotide substitutions (i.e., deletions, insertions, substitutions, or addition of one or more nucleotides in the sequence) which do not effect (in particular impair) the yeast promoter activity. Regions that can be modified without significantly affecting the yeast promoter activity can be identified by deletion studies. A mutant promoter of the present invention has at least about 20%, preferably at least about 40%, more preferably at least about 60%, more preferably at least about 80%, more preferably at least about 90%, more preferably at least about 100%, more preferably at least about 200%, more preferably at least about 300% and most preferably at least about 500% of the promoter activity of the Yarrowia GPD promoter region described herein as SEQ ID NO:2.

U.S. Pat. No. 7,259,255 describes a variety of methods for mutagenesis, suitable for the generation of mutant promoters. This would permit production of a putative promoter having, for example, a more desirable level of promoter activity in the host cell or a more desirable sequence for purposes of cloning (e.g., removal of a restriction enzyme site within the native promoter region). Similarly, the cited reference also discusses means to examine regions of a nucleotide of interest important for promoter activity (i.e., functional analysis via deletion mutagenesis to determine the minimum portion of the putative promoter necessary for activity).

All variant promoter regions of a gpd Yarrowia gene, derived from the promoter regions described herein, are within the scope of the present disclosure.

Similarly, it should be noted that one could isolate regions upstream of the GPD initiation codon in various Yarrowia species and strains, other than the region isolated in U.S. Pat. No. 7,259,255 from Yarrowia lipolytica ATCC #76982, and thereby identify alternate promoter regions of a gpd Yarrowia gene. As is well known in the art, isolation of homologous promoter regions or genes using sequence-dependent protocols is readily possible using various techniques (see, U.S. Pat. No. 7,259,255). Examples of sequence-dependent protocols useful to isolate homologous promoter regions include, but are not limited to: 1) methods of nucleic acid hybridization; 2) methods of DNA and RNA amplification, as exemplified by various uses of nucleic acid amplification technologies [e.g., polymerase chain reaction [“PCR”], Mullis et al., U.S. Pat. No. 4,683,202; ligase chain reaction [“LCR”], Tabor, S. et al., Proc. Acad. Sci. U.S.A., 82:1074 (1985); or strand displacement amplification (SDA), Walker, et al., Proc. Natl. Acad. Sci. U.S.A., 89:392 (1992)]; and 3) methods of library construction and screening by complementation. Based on sequence conservation between related organisms, one would expect that the promoter regions would likely share significant homology (i.e., at least about 70% identity, more preferably at least about 85% identity and more preferably at least about 95% identity); however, one or more differences in nucleotide sequence could be observed when aligned with promoter regions of comparable length derived from the upstream region of SEQ ID NO:1. For example, one of skill in the art could readily isolate the Yarrowia GPD promoter region from Y. lipolytica ATCC #20362, Y. lipolytica ATCC #20510, Y. lipolytica ATCC #8661 or Y. lipolytica ATCC #20228. Similarly, the following strains of Yarrowia lipolytica could be obtained from the Herman J. Phaff Yeast Culture Collection, University of California Davis (Davis, Calif.): Y. lipolytica 49-14, Y. lipolytica 49-49, Y. lipolytica 50-140, Y. lipolytica 50-46, Y. lipolytica 50-47, Y. lipolytica 51-30, Y. lipolytica 60-26, Y. lipolytica 70-17, Y. lipolytica 70-18, Y. lipolytica 70-19, Y. lipolytica 70-20, Y. lipolytica 74-78, Y. lipolytica 74-87, Y. lipolytica 74-88, Y. lipolytica 74-89, Y. lipolytica 76-72, Y. lipolytica 76-93, Y. lipolytica 77-12T and Y. lipolytica 77-17. Or, strains could be obtained from the Laboratoire de Microbiologie et Génétique Moléculaire of Dr. Jean-Marc Nicaud, INRA Centre de Grignon, France, including for example, Yarrowia lipolytica JMY798 (Mli\check{c}ková, K. et al., Appl Environ Microbiol. 70 (7):3918-24 (2004)), Y. lipolytica JMY399 (Barth, G., and C. Gaillardin. In, Nonconventional Yeasts In Biotechnology; Wolf, W. K., Ed.; Springer-Verlag: Berlin, Germany, 1996; pp 313-388) and Y. lipolytica JMY154 (Wang, H. J., et al., J. Bacteriol. 181 (17):5140-8 (1999)).

In general, microbial expression systems and expression vectors containing regulatory sequences that direct high level expression of foreign proteins are well known to those skilled in the art. Any of these could be used to construct chimeric genes, which could then be introduced into appropriate microorganisms via transformation to provide high-level expression of the encoded enzymes.

Vectors (e.g., constructs, plasmids) and DNA expression cassettes useful for the transformation of suitable microbial host cells are well known in the art. The specific choice of sequences present in the construct is dependent upon the desired expression products, the nature of the host cell and the proposed means of separating transformed cells versus non-transformed cells. Typically, however, the vector contains at least one expression cassette, a selectable marker and sequences allowing autonomous replication or chromosomal integration. Suitable expression cassettes comprise a region 5′ of the gene that controls transcription (e.g., a promoter), the gene coding sequence, and a region 3′ of the DNA fragment that controls transcriptional termination, i.e., a terminator. It is most preferred when both control regions are derived from genes from the transformed yeast cell, although they need not be derived from genes native to the host.

Herein, transcriptional control regions (also initiation control regions or promoters) that are useful to drive expression of a coding gene of interest in the desired yeast cell are those promoter regions of a gpd Yarrowia gene, as described supra. Once the promoter regions are identified and isolated, they may be operably linked to a coding region of interest to create a chimeric gene. The chimeric gene may then be expressed in a suitable expression vector in transformed yeast cells, particularly in the cells of oleaginous yeast (e.g., Yarrowia lipolytica).

Coding regions of interest to be expressed in transformed yeast cells may be either endogenous to the host or heterologous. Genes encoding proteins of commercial value are particularly suitable for expression. For example, suitable coding regions of interest may include (but are not limited to) those encoding viral, bacterial, fungal, plant, insect, or vertebrate coding regions of interest, including mammalian polypeptides. Further, these coding regions of interest may be, for example, structural proteins, signal transduction proteins, transcription factors, enzymes (e.g., oxidoreductases, transferases, hydrolyases, lyases, isomerases, ligases), or peptides. A non-limiting list includes genes encoding enzymes such as acyltransferases, aminopeptidases, amylases, carbohydrases, carboxypeptidases, catalyases, cellulases, chitinases, cutinases, cyclodextrin glycosyltransferases, deoxyribonucleases, esterases, alpha (α)-galactosidases, beta (β)-glucanases, beta (β)-galactosidases, glucoamylases, alpha (α)-glucosidases, beta (β)-glucosidases, invertases, laccases, lipases, mannosidases, mutanases, oxidases, pectinolytic enzymes, peroxidases, phospholipases, phosphotases, phytases, polyphenoloxidases, proteolytic enzymes, ribonucleases, transglutaminases or xylanases.

In some embodiments here, preferred coding regions of interest are those encoding enzymes involved in the production of microbial oils, including omega-6 and omega-3 fatty acids (i.e., omega-6 and omega-3 fatty acid biosynthetic pathway enzymes). Thus, preferred coding regions include those encoding desaturases (e.g., delta-8 desaturases, delta-5 desaturases, delta-17 desaturases, delta-12 desaturases, delta-4 desaturases, delta-6 desaturases, delta-15 desaturases and delta-9 desaturases) and elongases (e.g., C_{14/16} elongases, C_{16/18} elongases, C_{18/20} elongases, C_{20/22} elongases, delta-6 elongases and delta-9 elongases).

More specifically, the omega-3/omega-6 fatty acid biosynthetic pathway is illustrated in FIG. 4. All pathways require the initial conversion of oleic acid [18:1] to linoleic acid [“LA”; 18:2], the first of the omega-6 fatty acids, by a delta-12 desaturase. Then, using the “delta-9 elongase/delta-8 desaturase pathway” and LA as substrate, long-chain omega-6 fatty acids are formed as follows: 1) LA is converted to eicosadienoic acid [“EDA”; 20:2] by a delta-9 elongase; 2) EDA is converted to dihomo-γ-linolenic acid [“DGLA”; 20:3] by a delta-8 desaturase; 3) DGLA is converted to arachidonic acid [“ARA”; 20:4] by a delta-5 desaturase; 4) ARA is converted to docosatetraenoic acid [“DTA”; 22:4] by a C_{20/22} elongase; and, 5) DTA is converted to docosapentaenoic acid [“DPAn-6”; 22:5] by a delta-4 desaturase.

The “delta-9 elongase/delta-8 desaturase pathway” can also use alpha-linolenic acid [“ALA”; 18:3] as substrate to produce long-chain omega-3 fatty acids as follows: 1) LA is converted to ALA, the first of the omega-3 fatty acids, by a delta-15 desaturase; 2) ALA is converted to eicosatrienoic acid [“ETrA”; 20:3] by a delta-9 elongase; 3) ETrA is converted to eicosatetraenoic acid [“ETA”; 20:4] by a delta-8 desaturase; 4) ETA is converted to eicosapentaenoic acid [“EPA”; 20:5] by a delta-5 desaturase; 5) EPA is converted to docosapentaenoic acid [“DPA”; 22:5] by a C_{20/22} elongase; and, 6) DPA is converted to docosahexaenoic acid [“DHA”; 22:6] by a delta-4 desaturase. Optionally, omega-6 fatty acids may be converted to omega-3 fatty acids. For example, ETA and EPA are produced from DGLA and ARA, respectively, by delta-17 desaturase activity.

Alternate pathways for the biosynthesis of ω-3/ω-6 fatty acids utilize a delta-6 desaturase and C_{18/20} elongase, that is, the “delta-6 desaturase/delta-6 elongase pathway”. More specifically, LA and ALA may be converted to GLA and stearidonic acid [“STA”; 18:4], respectively, by a delta-6 desaturase; then, a C_{18/20} elongase converts GLA to DGLA and/or STA to ETA. Downstream PUFAs are subsequently formed as described above.

Thus, one aspect of the present disclosure provides a chimeric gene comprising a Yarrowia GPD promoter region, as well as recombinant expression vectors comprising the chimeric gene.

Also provided herein is a method for the expression of a coding region of interest in a transformed yeast cell comprising:
a) providing the transformed yeast cell having a chimeric gene, wherein the chimeric gene comprises:
   (1) a promoter region of a gpd Yarrowia gene; and,
   (2) the coding region of interest which is expressible in the yeast cell;
wherein the promoter region is operably linked to the coding region of interest; and,
b) growing the transformed yeast cell of step (a) under conditions whereby the chimeric gene of step (a) is expressed.
   The polypeptide so produced by expression of the chimeric gene may optionally be recovered from the culture.

One of skill in the art will appreciate that the disclosure herein also provides a method for the production of an omega-3 fatty acid or omega-6 fatty acid comprising:
a) providing a transformed oleaginous yeast comprising a chimeric gene, wherein the chimeric gene comprises:
   i) a promoter region of a gpd Yarrowia gene; and,
   ii) a coding region encoding at least one omega-3 fatty acid or omega-6 fatty acid biosynthetic pathway enzyme;
   wherein the promoter region and the coding region are operably linked; and,
b) growing the transformed oleaginous yeast of step (a) under conditions whereby the at least one omega-3 fatty acid or omega-6 fatty acid biosynthetic pathway enzyme is expressed and the omega-3 fatty acid or the omega-6 fatty acid is produced; and,
c) optionally recovering the omega-3 fatty acid or the omega-6 fatty acid.
   The omega-3 fatty acid or the omega-6 fatty acid may be selected from the group consisting of: LA, GLA, EDA, DGLA, ARA, DTA, DPAn-6, ALA, STA, ETrA, ETA, EPA, DPAn-3 and DHA.

Once a DNA cassette (e.g., comprising a chimeric gene comprising a promoter region of a gpd Yarrowia gene, ORF and terminator) suitable for expression in a yeast cell has been obtained, it is placed in a plasmid vector capable of autonomous replication in the yeast cell, or it is directly integrated into the genome of the yeast cell. Integration of expression cassettes can occur randomly within the yeast genome or can be targeted through the use of constructs containing regions of homology with the yeast genome sufficient to target recombination to a specific locus. All or some of the transcriptional and translational regulatory regions can be provided by the endogenous locus where constructs are targeted to an endogenous locus.

Where two or more genes are expressed from separate replicating vectors, it is desirable that each vector has a different means of selection and should lack homology to the other construct(s) to maintain stable expression and prevent reassortment of elements among constructs. Judicious choice of regulatory regions, selection means and method of propagation of the introduced construct(s) can be experimentally determined so that all introduced chimeric genes are expressed at the necessary levels to provide for synthesis of the desired products.

U.S. Pat. No. 7,259,255 describes means to increase expression of a particular coding region of interest.

Constructs comprising the chimeric gene(s) of interest may be introduced into a yeast cell by any standard technique. These techniques include transformation (e.g., lithium acetate transformation [Methods in Enzymology, 194:186-187 (1991)]), protoplast transformation, bolistic impact, electroporation, microinjection, or any other method that introduces the chimeric gene(s) of interest into the yeast cell.

For convenience, a yeast cell that has been manipulated by any method to take up a DNA sequence, for example, in an expression cassette, is referred to herein as “transformed”, “transformant” or “recombinant” (as these terms will be used interchangeably herein). The transformed yeast will have at least one copy of the expression construct and may have two or more, depending upon whether the expression cassette is integrated into the genome or is present on an extrachromosomal element having multiple copy numbers.

The transformed yeast cell can be identified by various selection techniques, as described in U.S. Pat. No. 7,238,482, U.S. Pat. No. 7,259,255 and U.S. Pat. Pub No. 2006-0115881-A1.

Following transformation, substrates upon which the translated products of the chimeric genes act may be produced by the yeast either naturally or transgenically, or they may be provided exogenously.

Yeast cells for expression of the instant chimeric genes comprising a promoter region of a gpd Yarrowia gene may include yeast that grow on a variety of feedstocks, including simple or complex carbohydrates, fatty acids, organic acids, oils, glycerol and alcohols, and/or hydrocarbons over a wide range of temperature and pH values. It is contemplated that because transcription, translation and the protein biosynthetic apparatus are highly conserved, any yeast will be a suitable host for expression of the present chimeric genes.

As previously noted, yeast do not form a specific taxonomic or phylogenetic grouping, but instead comprise a diverse assemblage of unicellular organisms that occur in the Ascomycotina and Basidiomycotina, most of which reproduce by budding (or fission) and derive energy via fermentation processes. Examples of some yeast genera include, but are not limited to: Agaricostilbum, Ambrosiozyma, Arthroascus, Arxula, Ashbya, Babjevia, Bensingtonia, Botryozyma, Brettanomyces, Bullera, Candida, Clavispora, Cryptococcus, Cystofilobasidium, Debaryomyces, Dekkera, Dipodascus, Endomyces, Endomycopsella, Erythrobasidium, Fellomyces, Filobasidium, Galactomyces, Geotrichum, Guilliermondella, Hansenula, Hanseniaspora, Kazachstania, Kloeckera, Kluyveromyces, Kockovaella, Kodamaea, Komagataella, Kondoa, Lachancea, Leucosporidium, Leucosporidiella, Lipomyces, Lodderomyces, Issatchenkia, Magnusiomyces, Mastigobasidium, Metschnikowia, Monosporella, Myxozyma, Nadsonia, Nematospora, Oosporidium, Pachysolen, Pichia, Phaffia, Pseudozyma, Reniforma, Rhodosporidium, Rhodotorula, Saccharomyces, Saccharomycodes, Saccharomycopsis, Saturnispora, Schizoblastosporion, Schizosaccharomyces, Sirobasidium, Smithiozyma, Sporobolomyces, Sporopachydermia, Starmerella, Sympodiomycopsis, Sympodiomyces, Torulaspora, Tremella, Trichosporon, Trichosporiella, Trigonopsis, Udeniomyces, Wickerhamomyces, Williopsis, Xanthophyllomyces, Yarrowia, Zygosaccharomyces, Zygotorulaspora, Zymoxenogloea and Zygozyma.

In preferred embodiments, the transformed yeast is an oleaginous yeast. These organisms are naturally capable of oil synthesis and accumulation, wherein the oil can comprise greater than about 25% of the cellular dry weight, more preferably greater than about 30% of the cellular dry weight, and most preferably greater than about 40% of the cellular dry weight. Genera typically identified as oleaginous yeast include, but are not limited to: Yarrowia, Candida, Rhodotorula, Rhodosporidium, Cryptococcus, Trichosporon and Lipomyces. More specifically, illustrative oil-synthesizing yeasts include: Rhodosporidium toruloides, Lipomyces starkeyii, L. lipoferus, Candida revkaufi, C. pulcherrima, C. tropicalis, C. utilis, Trichosporon pullans, T. cutaneum, Rhodotorula glutinus, R. graminis, and Yarrowia lipolytica (formerly classified as Candida lipolytica). Alternately, oil biosynthesis may be genetically engineered such that the transformed yeast can produce more than 25% oil of the cellular dry weight, and thereby be considered oleaginous.

Most preferred is the oleaginous yeast Yarrowia lipolytica. In a further embodiment, most preferred are the Y. lipolytica strains designated as ATCC #20362, ATCC #8862, ATCC #18944, ATCC #76982 and/or LGAM S(7)1 (Papanikolaou S., and Aggelis G., Bioresour. Technol., 82 (1):43-9 (2002)). The Y. lipolytica strain designated as ATCC #76982 was the particular strain from which the gpd Yarrowia gene and promoter regions encompassed within SEQ ID NO:1 were isolated.

Specific teachings applicable for transformation of oleaginous yeasts (i.e., Yarrowia lipolytica) via integration techniques based on linearized fragments of DNA include U.S. Pat. No. 4,880,741 and U.S. Pat. No. 5,071,764 and Chen, D. C. et al. (Appl. Microbiol. Biotechnol., 48 (2):232-235 (1997)). Specific teachings applicable for expression of omega-3 fatty acid or omega-6 fatty acid biosynthetic pathway enzymes in the oleaginous yeast Y. lipolytica are described in U.S. Pat. 7,238,482, U.S. Pat. No. 7,550,286, U.S. Pat. No. 7,588,931, U.S. Pat. Pub No. 2006-0115881-A1, U.S. Pat. Pub No. 2009-0093543-A1, and U.S. patent application Ser. No. 12/814,815 (filed Jun. 14, 2010 and having Attorney Docket No. CL4674USNA), each incorporated herein by reference in their entirety.

The transformed yeast cell is grown under conditions that optimize expression of the chimeric gene(s). In general, media conditions may be optimized by modifying the type and amount of carbon source, the type and amount of nitrogen source, the carbon-to-nitrogen ratio, the amount of different mineral ions, the oxygen level, growth temperature, pH, length of the biomass production phase, length of the oil accumulation phase and the time and method of cell harvest. Microorganisms of interest, such as oleaginous yeast (e.g., Yarrowia lipolytica) are generally grown in a complex medium such as yeast extract-peptone-dextrose broth [“YPD”] or a defined minimal media that lacks a component necessary for growth and thereby forces selection of the desired expression cassettes (e.g., Yeast Nitrogen Base (DIFCO Laboratories, Detroit, Mich.)).

Fermentation media suitable for the transformed yeast described herein must contain a suitable carbon source. Suitable carbon sources may include, but are not limited to: monosaccharides, disaccharides, oligosaccharides, polysaccharides, sugar alcohols, mixtures from renewable feedstocks, alkanes, fatty acids, esters of fatty acids, monoglycerides, diglycerides, triglycerides, phospholipids, various commercial sources of fatty acids, and one-carbon sources, such as are described in U.S. Pat. No. 7,259,255. Hence it is contemplated that the source of carbon utilized may encompass a wide variety of carbon-containing sources and will only be limited by the choice of the yeast species. Although all of the above mentioned carbon sources and mixtures thereof are expected to be suitable herein, preferred carbon sources are sugars (e.g., glucose, invert sucrose, sucrose, fructose and combinations thereof), glycerols, and/or fatty acids (see U.S. patent application Ser. No. 12/641,929 (filed Dec. 19, 2009 and having Attorney Docket No. CL2233USCIP).

Nitrogen may be supplied from an inorganic (e.g., (NH₄)₂SO₄) or organic (e.g., urea or glutamate) source. In addition to appropriate carbon and nitrogen sources, the fermentation media must also contain suitable minerals, salts, cofactors, buffers, vitamins and other components known to those skilled in the art suitable for the growth of the transformed yeast (and optionally, promotion of the enzymatic pathways necessary for omega-3/omega-6 fatty acid production). Particular attention is given to several metal ions, such as Fe⁺², Cu⁺², Mn⁺², Co⁺², Zn⁺² and Mg⁺², that promote synthesis of lipids and PUFAs (Nakahara, T. et al., Ind. Appl. Single Cell Oils, D. J. Kyle and R. Colin, eds. pp 61-97 (1992)).

Preferred growth media for the methods and transformed yeast cells described herein are common commercially prepared media, such as Yeast Nitrogen Base (DIFCO Laboratories, Detroit, Mich.). Other defined or synthetic growth media may also be used and the appropriate medium for growth of the transformant host cells will be known by one skilled in the art of microbiology or fermentation science. A suitable pH range for the fermentation is typically between about pH 4.0 to pH 8.0, wherein pH 5.5 to pH 7.5 is preferred as the range for the initial growth conditions. The fermentation may be conducted under aerobic or anaerobic conditions, wherein microaerobic conditions are preferred.

Typically, accumulation of high levels of omega-3/omega-6 fatty acids in oleaginous yeast cells requires a two-stage process, since the metabolic state must be “balanced” between growth and synthesis/storage of fats. Thus, most preferably, a two-stage fermentation process is necessary for the production of omega-3/omega-6 fatty acids in oleaginous yeast (e.g., Yarrowia lipolytica). This approach is described in U.S. Pat. No. 7,238,482.

Host cells comprising a suitable coding region of interest operably linked to promoter regions of a gpd Yarrowia gene may be cultured using methods known in the art. For example, the cell may be cultivated by shake flask cultivation or small-/large-scale fermentation in laboratory or industrial fermentors performed in a suitable medium and under conditions allowing expression of the coding region of interest. Similarly, where commercial production of a product that relies on the instant genetic chimera is desired, a variety of culture methodologies may be applied. For example, large-scale production of a specific gene product over-expressed from a recombinant host may be produced by a batch, fed-batch or continuous fermentation process (see U.S. Pat. No. 7,259,255).

### EXAMPLES

The present invention is further described in the following Examples, which illustrate reductions to practice of the invention but do not completely define all of its possible variations.

### General Methods

Standard recombinant DNA and molecular cloning techniques used in the Examples are well known in the art and are described by: 1) Sambrook, J., Fritsch, E. F. and Maniatis, T., Molecular Cloning: A Laboratory Manual; Cold Spring Harbor Laboratory: Cold Spring Harbor, N.Y. (1989) (Maniatis); 2) T. J. Silhavy, M. L. Bennan, and L. W. Enquist, Experiments with Gene Fusions; Cold Spring Harbor Laboratory: Cold Spring Harbor, N.Y. (1984); and 3) Ausubel, F. M. et al., Current Protocols in Molecular Biology, published by Greene Publishing Assoc. and Wiley-Interscience (1987).

Materials and methods suitable for the maintenance and growth of microbial cultures are well known in the art. Techniques suitable for use in the following examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, Eds), American Society for Microbiology: Washington, D.C. (1994)); or by Thomas D. Brock in Biotechnology: A Textbook of Industrial Microbiology, 2^{nd} ed., Sinauer Associates: Sunderland, Mass. (1989). All reagents, restriction enzymes and materials used for the growth and maintenance of microbial cells were obtained from Aldrich Chemicals (Milwaukee, Wis.), DIFCO Laboratories (Detroit, Mich.), GIBCO/BRL (Gaithersburg, Md.), New England Biolabs (Ipswich, Mass.), or Sigma Chemical Company (St. Louis, Mo.), unless otherwise specified. E. coli strains were typically grown at 37° C. on Luria Bertani [“LB”] plates.

General molecular cloning was performed according to standard methods (Sambrook et al., supra). DNA sequence was generated on an ABI Automatic sequencer using dye terminator technology (U.S. Pat. No. 5,366,860; EP 272,007) using a combination of vector and insert-specific primers. Sequence editing was performed in Sequencher (Gene Codes Corporation, Ann Arbor, Mich.). All sequences represent coverage at least two times in both directions. Comparisons of genetic sequences were accomplished using DNASTAR software (DNASTAR Inc., Madison, Wis.).

The meaning of abbreviations is as follows: “sec” means second(s), “min” means minute(s), “h” means hour(s), “d” means day(s), “μL” means microliter(s), “mL” means milliliter(s), “L” means liter(s), “μM” means micromolar, “mM” means millimolar, “M” means molar, “mmol” means millimole(s), “μmole” mean micromole(s), “g” means gram(s), “μg” means microgram(s), “ng” means nanogram(s), “U” means unit(s), “bp” means base pair(s) and “kB” means kilobase(s).

Nomenclature For Expression Cassettes: The structure of an expression cassette will be represented by a simple notation system of “X::Y::Z”, wherein X describes the promoter fragment, Y describes the gene fragment, and Z describes the terminator fragment, which are all operably linked to one another.

Transformation And Cultivation Of Yarrowia lipolytica: Y. lipolytica strains with ATCC Accession Nos. #20362, #76982 and #90812 were purchased from the American Type Culture Collection (Rockville, Md.). Yarrowia lipolytica strains were typically grown at 28-30° C. Basic Minimal Media [“MM”] (per liter) includes: 20 g glucose, 1.7 g yeast nitrogen base without amino acids, 1.0 g proline, and pH 6.1 (do not need to adjust). Agar plates were prepared as required by addition of 20 g/L agar to the liquid media, according to standard methodology.

Transformation of Y. lipolytica was performed as described in U.S. Pat. Appl. Pub. No. 2009-0093543-A1, hereby incorporated herein by reference.

### Example 1

### Isolation Of A Yarrowia lipolytica GPD Promoter Region

U.S. Pat. No. 7,259,255 describes: 1) the identification of a portion of the Yarrowia lipolytica gene encoding glyceraldehyde-3-phosphate dehydrogenase [“GPD”], by use of primers derived from conserved regions of other GPD sequences; 2) the use of a genome-walking technique to isolate the 5′ upstream region of the Yarrowia gpd gene; 3) the identification of a single 2316 bp contig comprising 1525 bp upstream of the GPD initiation codon and 791 bp of the gpd gene (SEQ ID NO:1; FIG. 1), wherein the gene was also found to comprise an intron (base pairs +49 to +194); and, 4) the identification of a putative GPD promoter region which was designated as “GPDPro” (SEQ ID NO:2) and which corresponded to the nucleotide region between the −968 position and the ‘ATG’ translation initiation site of the gpd gene (i.e., the −968 to −1 upstream region of the gpd gene and the +1 to +3 region of the gpd gene, wherein the ‘A’ nucleotide of the ‘ATG’ translation initiation codon was designated as +1).

### Example 2

### Construction Of A Modified Yarrowia lipolytica Promoter Region: The GPD-C Promoter (SEQ ID NO:3)

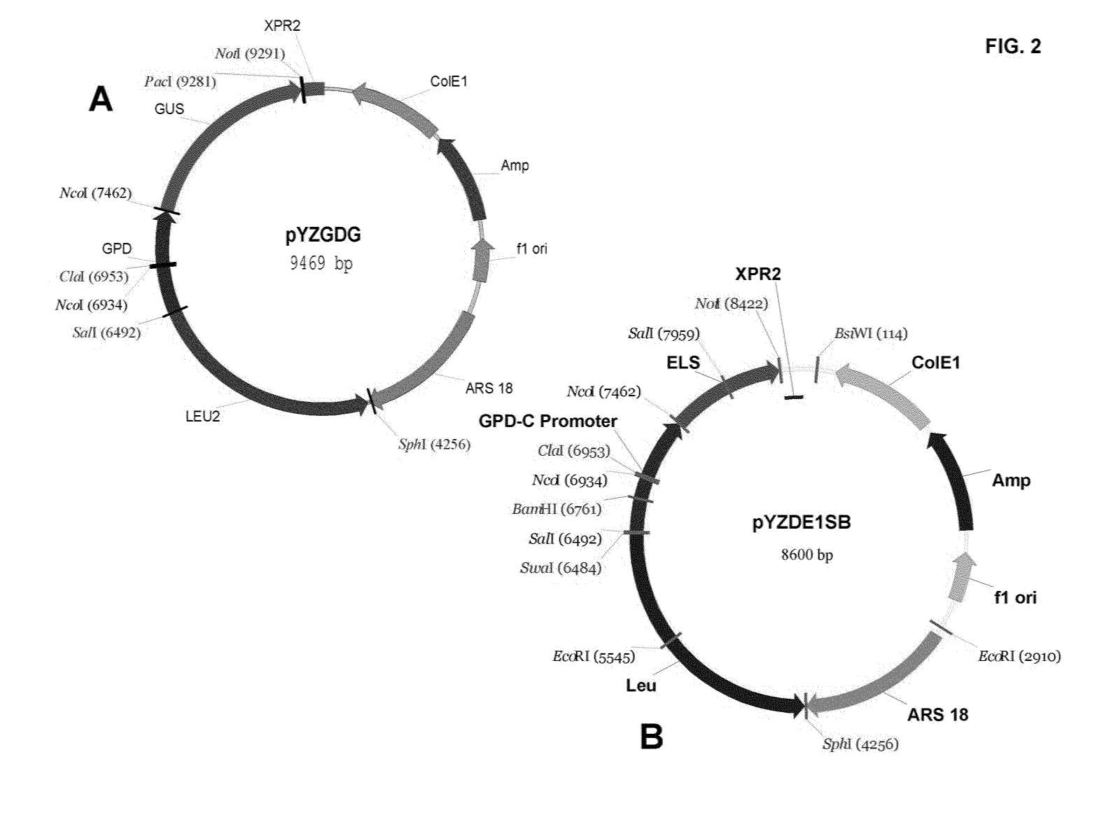

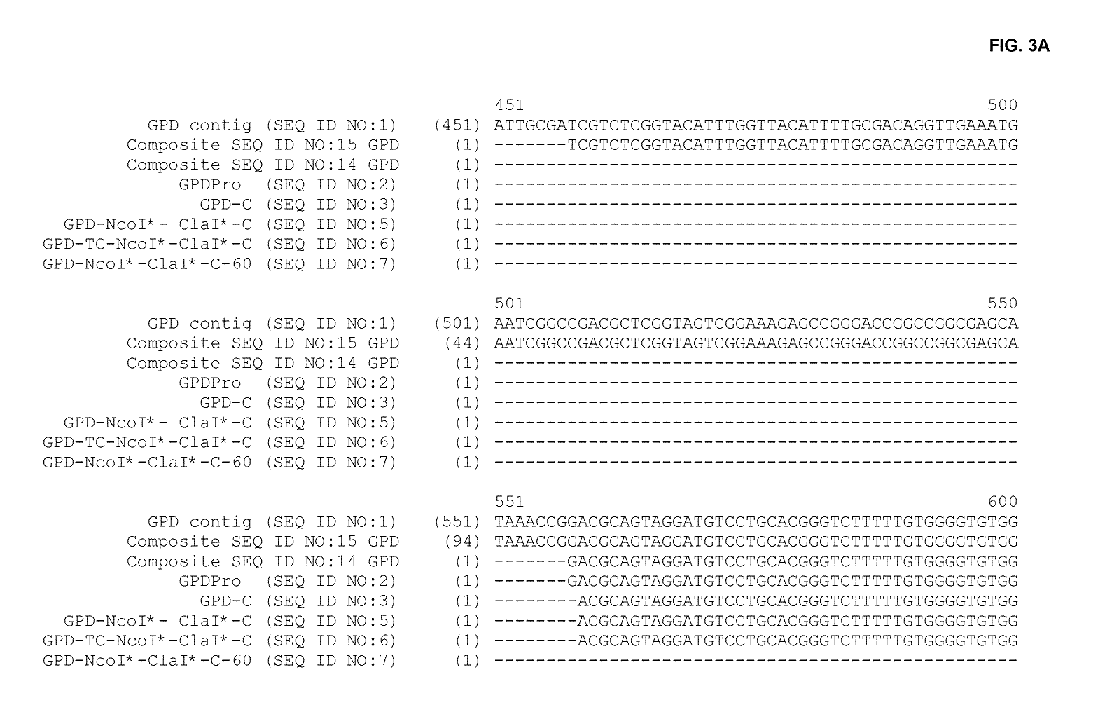

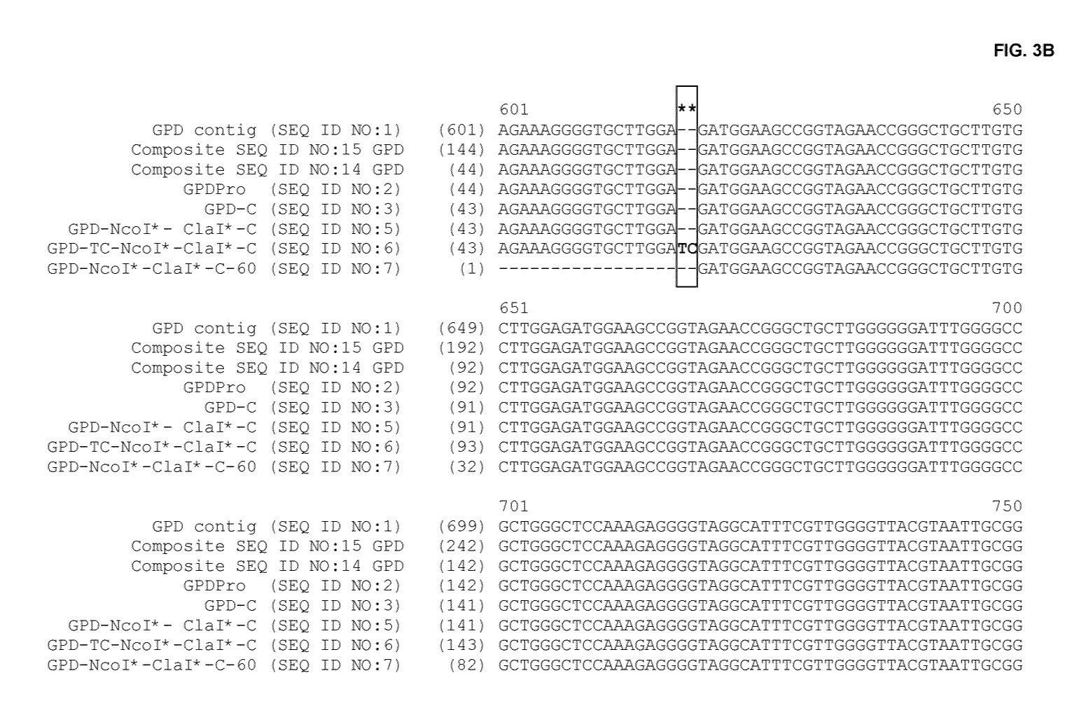

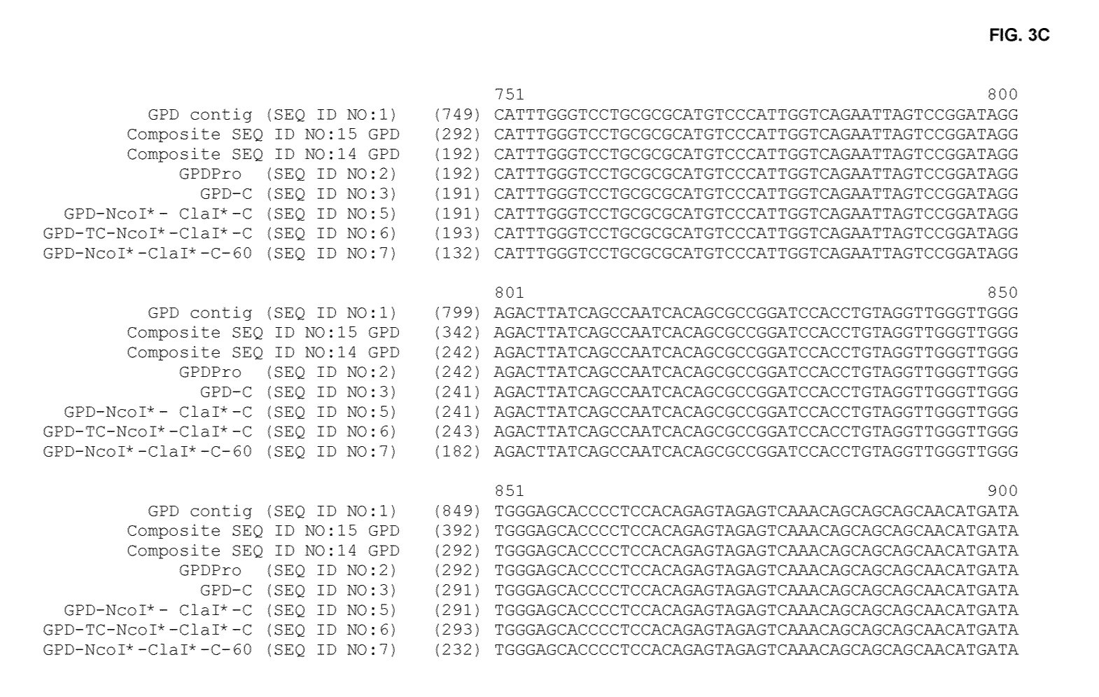

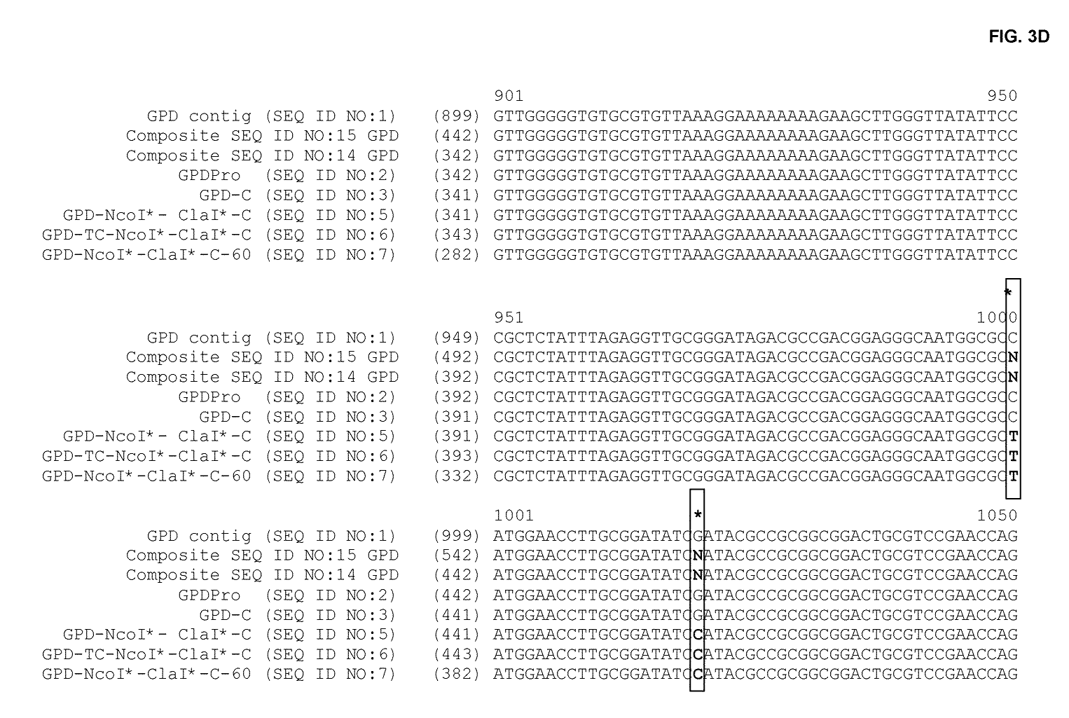

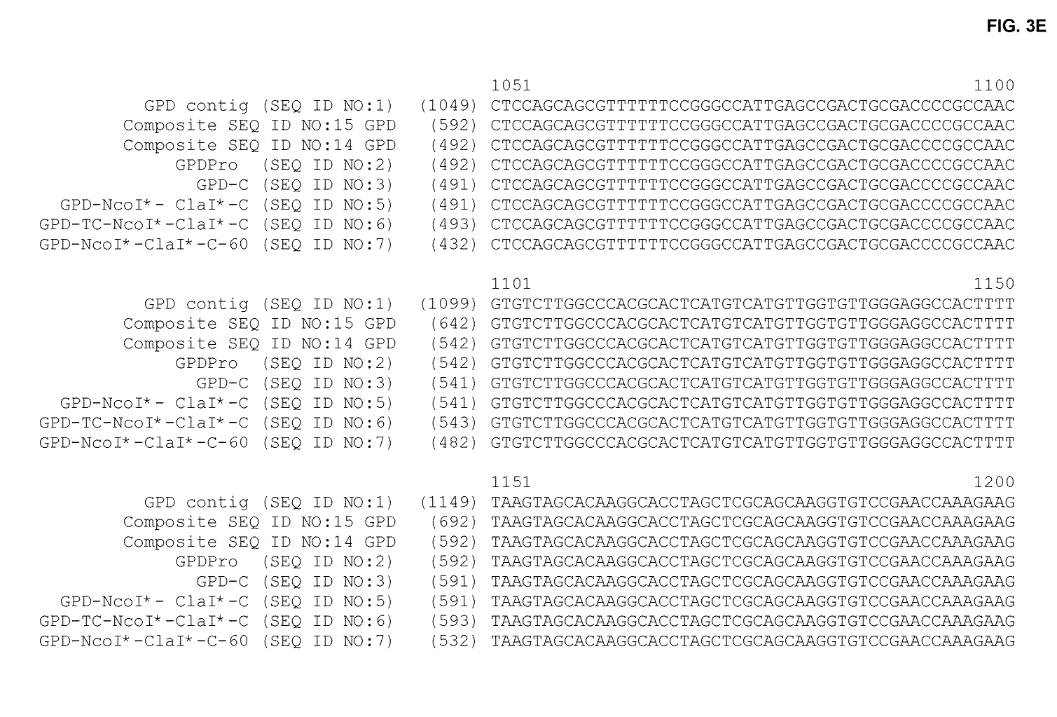

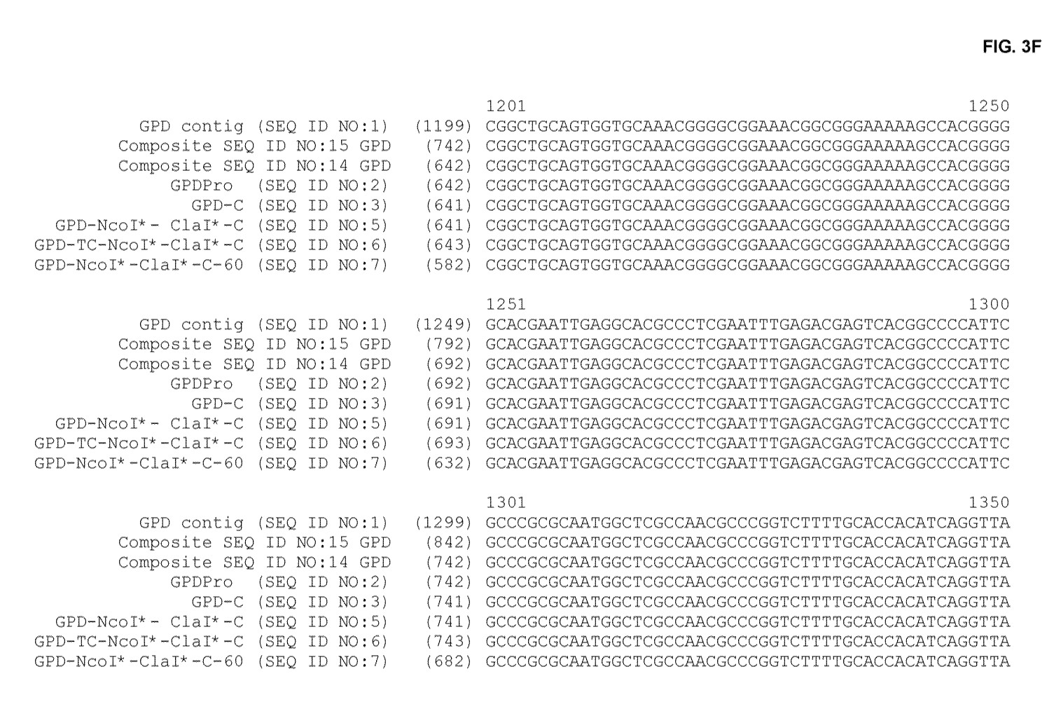

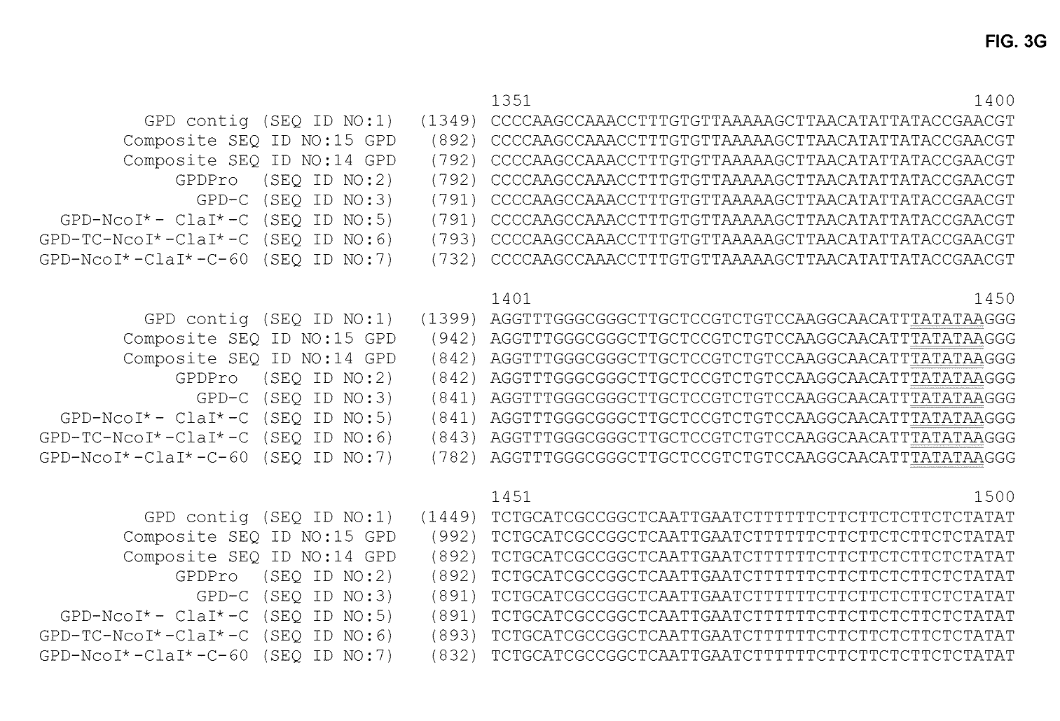

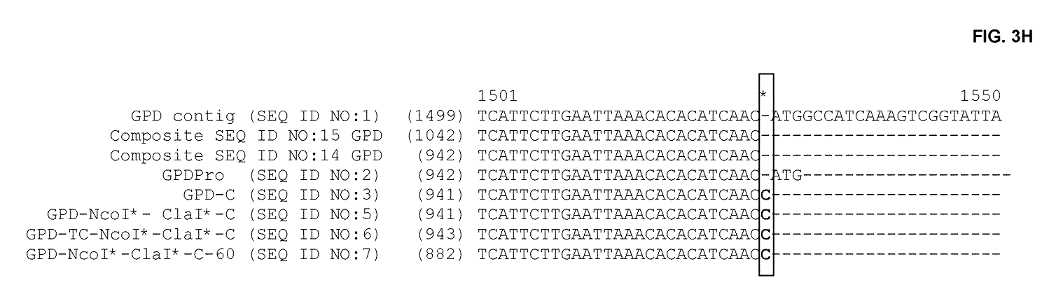

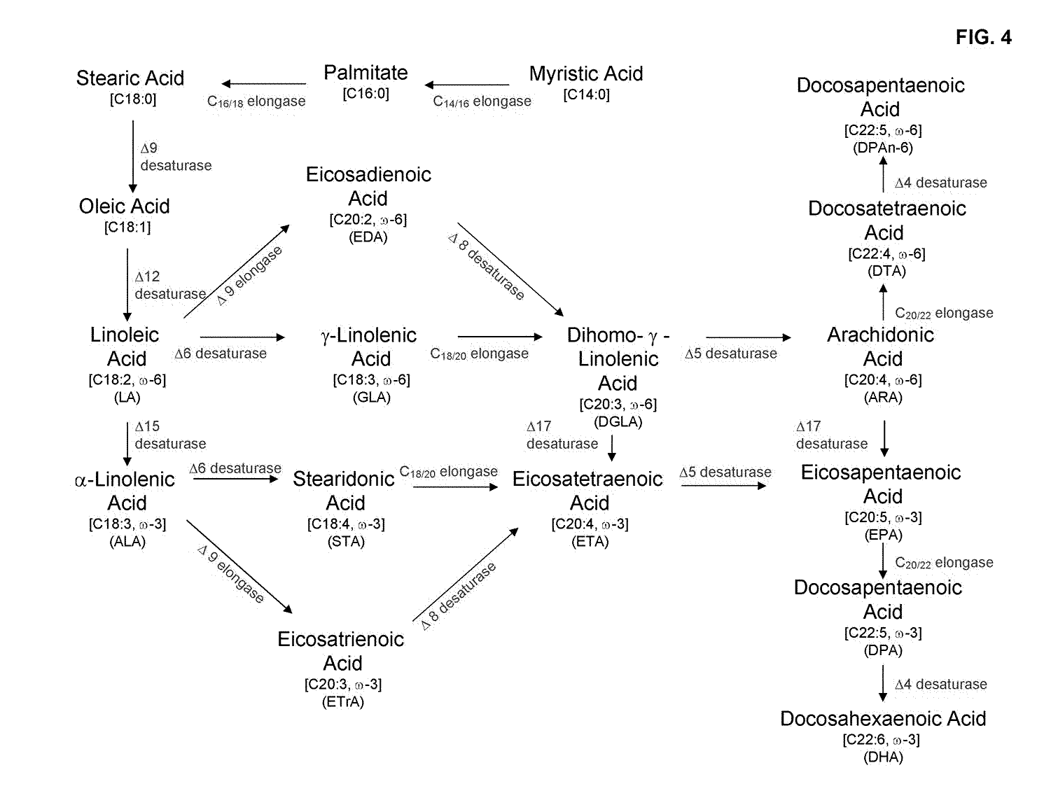

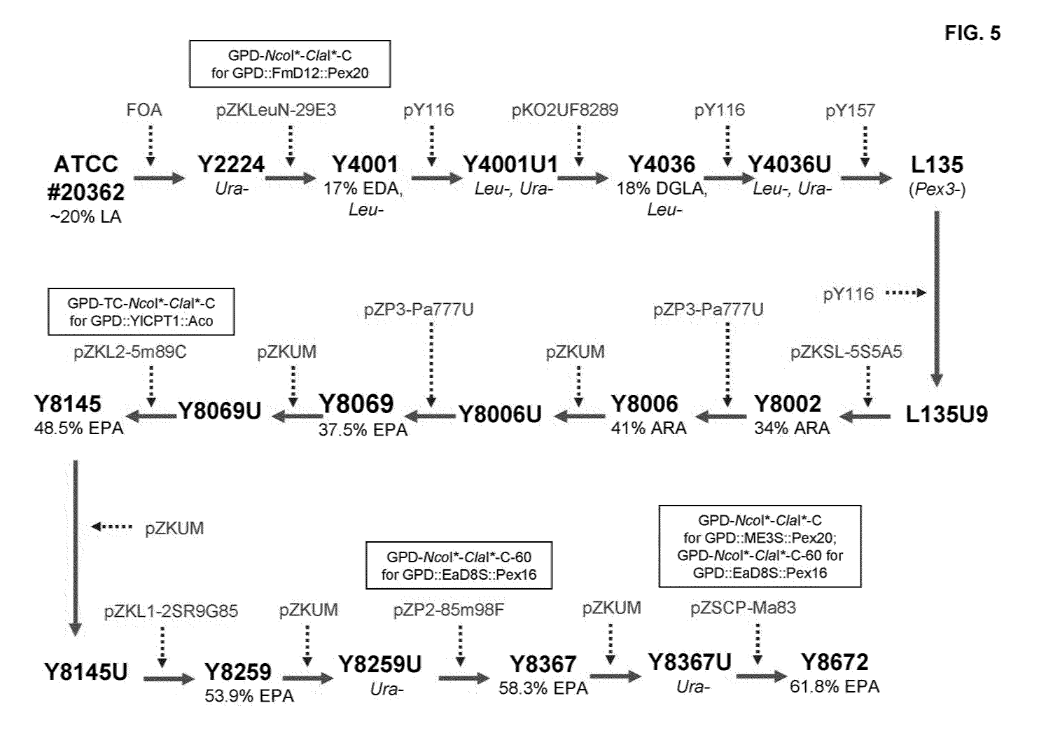

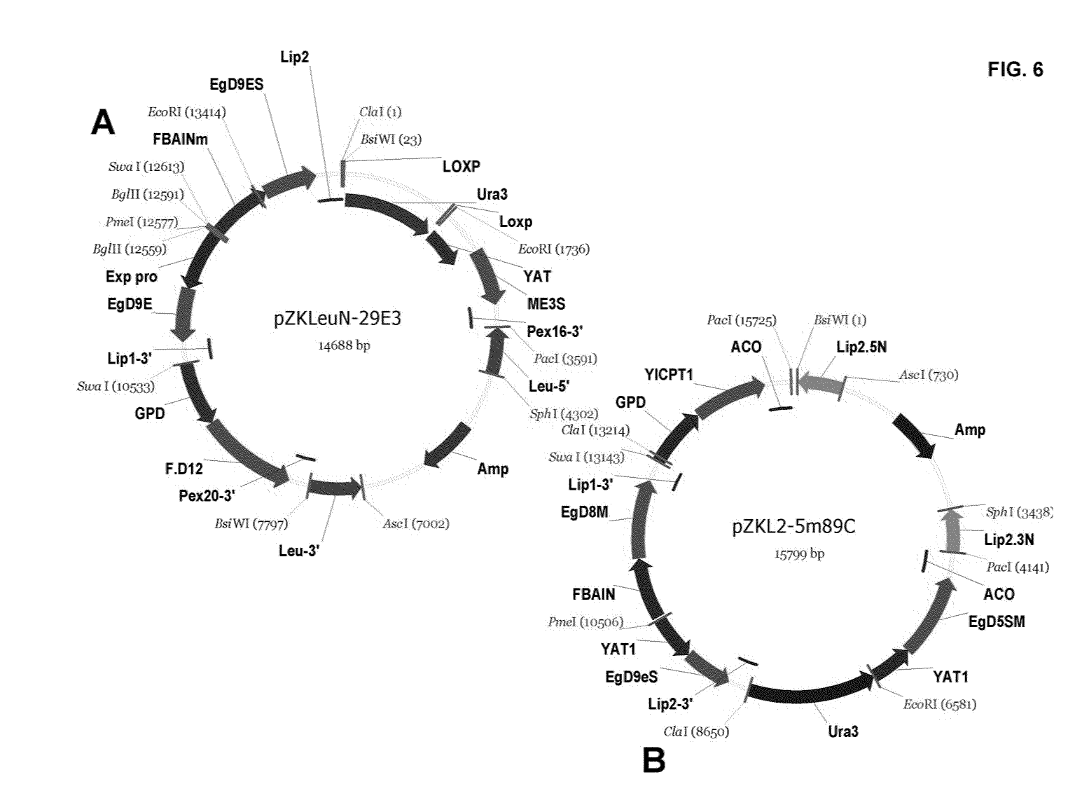

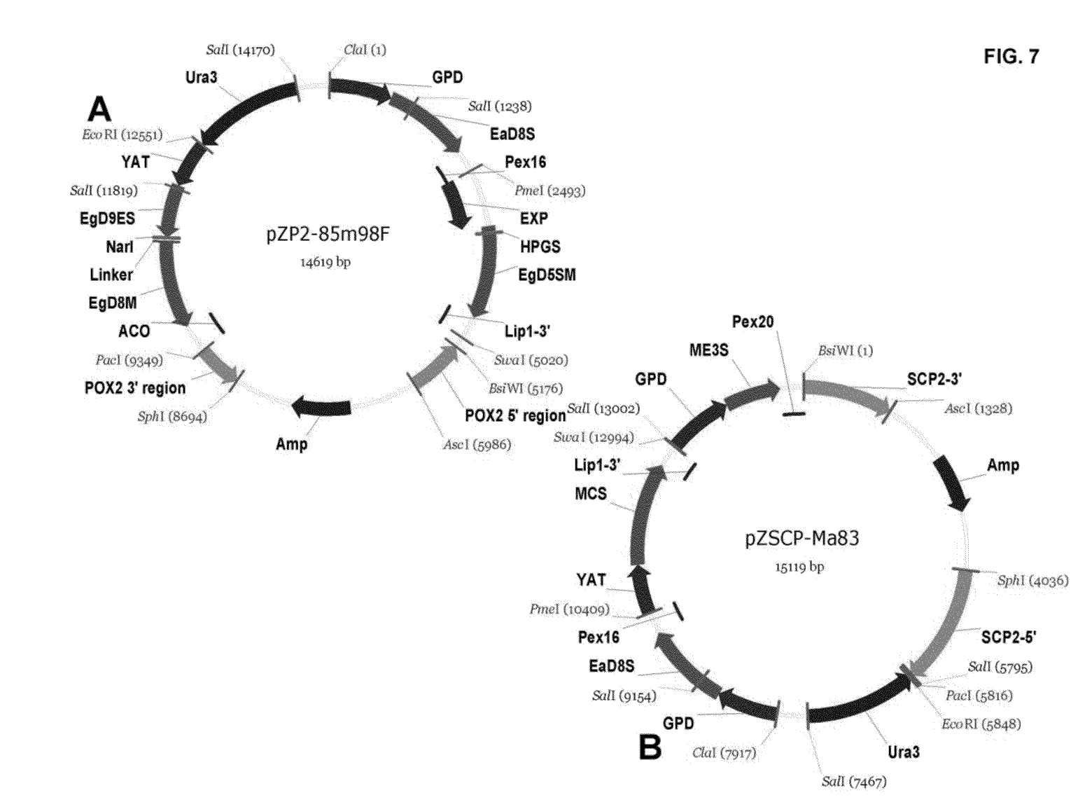

U.S. Pat. No. 7,259,255 also describes construction of plasmid “pYZGDG” (FIG. 2A herein), which contained a chimeric GPD::GUS::XPR gene comprising a Yarrowia GPD promoter, the E. coli reporter gene encoding β-glucuronidase [“GUS”] (Jefferson, R. A. Nature, 342 (6251):837-838 (1989)), and XPR terminator. Specifically, the putative GPDPro promoter region of Example 1 was amplified by PCR and then the reaction was purified using a Qiagen PCR purification kit. The resulting GPD product was then completely digested with SalI and subsequently partially digested with NcoI. The SalI/NcoI fragment was purified following gel electrophoresis in 1% (w/v) agarose and ligated to NcoI/SalI-digested pY5-30 vector (described in detail in Example 4 of U.S. Pat. No. 7,259,255) (wherein the NcoI/SalI digestion had excised the TEF promoter from the pY5-30 vector backbone).

The present Example herein clarifies that the Yarrowia GPD promoter region within the GPD::GUS::XPR chimeric gene of plasmid pYZGDG corresponded to a modified variant of the sequence set forth as SEQ ID NO:2, although this was not appreciated until preparation of the present application. Specifically, the Yarrowia GPD promoter region in plasmid pYZGDG corresponded to a 969 bp modified GPD-C promoter sequence set forth herein as SEQ ID NO:3. The GPD-C promoter differs from the GPD promoter of SEQ ID NO:2 in that it comprises a C insertion at +969 and the ATG at +969 to +971 of SEQ ID NO:2 are deleted. This modification optimized the translation initiation motif around the ‘ATG’ translation initiation site (details provided infra) and created a NcoI site for the cloning methodology used to produce pYZGDG. The sequence of plasmid pYZGDG is set forth herein as SEQ ID NO:4.

### Expression Of A Modified Yarrowia GPD Promoter: GPD-C (SEQ ID NO:3)

U.S. Pat. No. 7,259,255 also describes the transformation of pYZGDG (SEQ ID NO:4) into Y. lipolytica ATCC #76982 and determination of the activity of the GPD-C promoter (SEQ ID NO:3) in transformed cells containing the pYZGDG construct, based on histochemical and fluorometric assays designed to measure activity of the GUS reporter gene. Activity was compared to that of the translation elongation factor EF1-α [“TEF”] protein promoter (U.S. Pat. No. 6,265,185). In brief, the results of assays showed that the GPD-C promoter in construct pYZGDG was active and its activity was stronger than the activity of the TEF promoter.

Example 8 of U.S. Pat. No. 7,259,255 further describes the use of the GPD-C promoter (SEQ ID NO:3) to drive expression of a Fusarium moniliforme strain M-8114 delta-15 desaturase [“FmD15” or “Fm1”] in Y. lipolytica. When expressed, the delta-15 desaturase is capable of converting the substrate, linoleic acid [“LA”; 18:2, ω-6], to α-linolenic acid [“ALA”; 18:3, ω-3]. Wildtype Y. lipolytica are unable to produce ALA since they lack any native delta-15 desaturase activity.

Based on the production of ALA in transformed Y. lipolytica host cells comprising the chimeric GPD-C::FmD15::XPR gene (as compared to wildtype Y. lipolytica that produced no ALA), it was concluded that the supposed “GPD” promoter contained within the construct was suitable to drive expression of heterologous PUFA biosynthetic pathway enzymes in oleaginous yeast cells such as Y. lipolytica. It is now appreciated that this promoter was GPD-C, as set forth in SEQ ID NO:3 herein.

### Example 3

### Construction Of Additional Modified Yarrowia lipolytica GPD Promoter Regions: GPD-NcoI*-ClaI*-C (SEQ ID NO:5), GPD-TC-NcoI*-ClaI*-C (SEQ ID NO:6) and GPD-NcoI*-ClaI*-C-60 (SEQ ID NO:7)

The present Example describes the creation of three additional modified Yarrowia GPD promoters (i.e., the GPD-NcoI*-ClaI*-C promoter [SEQ ID NO:5], the GPD-TC-NcoI*-ClaI*-C promoter [SEQ ID NO:6] and the GPD-NcoI*-ClaI*-C-60 promoter [SEQ ID NO:7]), derived from the GPD-C promoter (SEQ ID NO:3) described supra in Example 2.

More specifically, the GUS reporter gene was excised from pYZGDG (SEQ ID NO:4) by partial NcoI and complete NotI digestion and replaced with an elongase gene [“EL1S”] derived from Mortierella alpina (GenBank Accession No. AX464731) and codon-optimized for expression in Yarrowia lipolytica, to thereby create plasmid pYZDE1SB (SEQ ID NO:8; FIG. 2B). Plasmid pYZDE1SB was subjected to site-directed mutagenesis using a Stratagene kit (La Jolla, Calif.) and recommended protocols. Three additional modified GPD promoters were thus created, as described below in Table 3.

TABLE 3
Wildtype And Modified Yarrowia GPD Promoter Regions

| | | Mutations with Respect to SEQ ID NO: 2 | | |
|---|---|---|---|---|
| | | (Abbreviations: “T” is deoxythymidine, “C” | | Promoter Region |
| | SEQ ID | is deoxycytidine, “A” is deoxyadenosine | Promoter | With Respect to |
| Promoter | NO | and “G” is deoxyguanosine) | Length | gpd Gene* |
| Wildtype GPD | SEQ ID | NONE | 971 bp | Comprises the |
| promoter [“GPDPro”] | NO: 2 | | | −968 to +3 |
| | | | | region |
| Modified GPD-C | SEQ ID | C insertion at +969; | 969 bp | Comprises the |
| promoter | NO: 3 | ATG deletion at +969 to +971 of SEQ ID NO: 2 | | −968 −1 region |
| Modified GPD-Ncol*- | SEQ ID | Internal Ncol site (C/CATGG) mutated to CTATGG | | |
| Clal*-C promoter | NO: 5 | (C to T mutation at +441); | 969 bp | Comprises the |
| | | Internal Clal site (ST/CGAT) mutated to ATCCAT | | −968 −1 region |
| | | (G to C mutation at +461); | | |
| | | C insertion at +969; | | |
| | | ATG deletion at +969 to +971 of SEQ ID NO: 2 | | |
| Modified GPD-TC- | SEQ ID | TC insertion at +61; | 971 bp | Comprises the |
| Ncol*-Clal*-C promoter | NO: 6 | Internal Ncol site (C/CATGG) mutated to CTATGG | | −968 to −1 region |
| | | (C to T mutation at +441); | | |
| | | Internal Clal site (AT/CGAT) mutated to ATCCT | | |
| | | (G to C mutation at +461); | | |
| | | C insertion at +969; | | |
| | | ATG deletion at +969 to +971 of SEQ ID NO: 2 | | |
| Modified GPD-NCOL*- | SEQ ID | Deletion of +1 to +60; | 909 bp | Comprises the |
| Clal*-C-60 promoter | NO: 7 | Internal Ncol site (C/CATGG) mutated to CTATGG | | −908 to −1 region |
| | | (C to T mutation at +441); | | |
| | | Internal Clal site (AT/CGAT) mutated to ATCCAT | | |
| | | (G to C mutation at +461); | | |
| | | C insertion at +969; | | |
| | | ATG deletion at +969 to +971 of SEQ ID NO: 2 | | |

*Promoter region with respect to Yarrowia lipolytica gpd gene (SEQ ID NO: 1) is described based on nucleotide numbering such that the ‘A’ position of the ‘ATG’ translation initiation codon is designated as +1.

A portion of a multiple sequence alignment of these promoters (i.e., the GPD-C promoter [SEQ ID NO:3], GPD-NcoI*-ClaI*-C promoter [SEQ ID NO:5], GPD-TC-NcoI*-ClaI*-C promoter [SEQ ID NO:6] and GPD-NcoI*-ClaI*-C-60 promoter [SEQ ID NO:7]), as well as the wildtype GPDPro promoter (SEQ ID NO:2) which includes to the −968 to −1 region upstream of the Yarrowia gpd gene and the +1 to +3 region of the gpd gene, the composite SEQ ID NO:14 GPD promoter, the composite SEQ ID NO:15 promoter, and the originally isolated contig comprising 1525 nucleotides of 5′ upstream untranslated sequence and 791 bp of the Yarrowia gpd gene (SEQ ID NO:1) is shown in FIG. 3. The alignment was performed using default parameters [gap opening penalty=15, gap extension penalty=6.66, and gap separation penalty range=8] of Vector NTI®'s Advance 9.1.0 AlignX program (Invitrogen Corporation, Carlsbad, Calif.)].

### Expression Of Modified GPD Promoters: GPD-NcoI*-ClaI*-C (SEQ ID NO:5), GPD-TC-NcoI*-ClaI*-C (SEQ ID NO:6) And GPD-NcoI*-ClaI*-C-60 (SEQ ID NO:7)

Using standard cloning methodology, the resultant modified Yarrowia GPD promoters (i.e., GPD-NcoI*-ClaI*-C, GPD-TC-NcoI*-ClaI*-C and GPD-NcoI*-ClaI*-C-60) were operably linked to the coding regions of several different PUFA biosynthetic pathway genes and suitable terminators derived from Yarrowia in various plasmid vectors.

The various plasmid vectors were transformed separately into several different strains of Y. lipolytica derived from Y. lipolytica ATCC #20362 that had been previously engineered to produce the substrate appropriate for the introduced gene. Thus, e.g., a host producing suitable quantities of either LA or ALA was required to enable expression of an introduced delta-9 elongase, since the delta-9 elongase converts LA to EDA and/or ALA to ETrA. Similarly, a host producing suitable quantities of either EDA or ETrA was required to enable expression of an introduced delta-8 desaturase, since the delta-8 desaturase converts EDA to DGLA and/or ETrA to ETA. See, FIG. 4.

Single colonies from each transformation were streaked onto MM selection plates and grown at 30° C. for 24 to 48 hrs. A loop of cells from each MM selection plate was then inoculated into liquid MM at 30° C.; the cells were shaken at 250 rpm/min for 2 days, collected by centrifugation and lipids were extracted. Fatty acid methyl esters [“FAMEs”] were prepared by trans-esterification, and subsequently analyzed with a Hewlett-Packard 6890 GC, as described in U.S. Pat. Appl. Pub. No. 2009-0093543-A1.

The promoter activity of each of the mutant Yarrowia GPD promoters (i.e., GPD-NcoI*-ClaI*-C, GPD-TC-NcoI*-ClaI*-C and GPD-NcoI*-ClaI*-C-60) was determined based on the substrate conversion efficiency of the particular gene to which the promoter was operably linked. More specifically, the conversion efficiency refers to the efficiency by which a particular enzyme can convert substrate to product and was calculated according to the following formula: ([product]/[substrate+product])*100, where ‘product’ includes the immediate product and all products in the pathway derived from it.

The mutant promoter was deemed active if suitable substrate conversion was observed. Suitable conversion was determined by comparing with the substrate conversion observed in the untransformed, parent strain of Y. lipolytica.

Based on the above analyses, each of the modified Yarrowia GPD promoters (i.e., GPD-NcoI*-ClaI*-C [SEQ ID NO:5], GPD-TC-NcoI*-ClaI*-C [SEQ ID NO:6] and GPD-NcoI*-ClaI*-C-60 [SEQ ID NO:7]) was deemed active. Thus, the modified Yarrowia GPD promoters were demonstrated to sustain mutations in the active region (i.e., in the region corresponding to bases 1 to 968 of SEQ ID NO:2) that do not change the active status of the promoter.

Specifically, for GPD-NcoI*-ClaI*-C (SEQ ID NO:5), GPD-TC-NcoI*-ClaI*-C (SEQ ID NO:6) and GPD-NcoI*-ClaI*-C-60 (SEQ ID NO:7), a substitution at bp +441 from C to T (effectively removing the internal NcoI site from the promoter region) did not impair the active status of the mutant promoter. Similarly, these modified GPD promoters also tolerated a substitution at bp +461 from G to C (effectively removing the internal ClaI site from the promoter region). It is hypothesized that a substitution at bp +441 from C to G or from C to A or a substitution at bp +461 from G to A or from G to T would also result in a functional promoter.

The active status of the GPD-NcoI*-ClaI*-C, GPD-TC-NcoI*-ClaI*-C and GPD-NcoI*-ClaI*-C-60 promoters was also not impaired by a C insertion at bp +969. As described in U.S. Pat. No. 7,125,672, the preferred consensus sequence of the codon-optimized translation initiation site for optimal expression of genes in Y. lipolytica is ‘MAMMATGNHS’ (SEQ ID NO:9), wherein the nucleic acid degeneracy code used is as follows: M=A/C; S=C/G; H=A/C/T; and N=A/C/G/T. While the four nucleotides immediately proceeding the ‘ATG’ translation initiation site are ‘CAAC’ in the wildtype Yarrowia GPD promoter set forth as SEQ ID NO:2 (therefore corresponding to the preferred consensus sequence), the C insertion at bp +969 in the modified GPD promoters results in a more preferred sequence of ‘AACC’ immediately upstream of the ‘ATG’ translation initiation site. In addition to the above modifications, the GPD-TC-NcoI*-ClaI*-C promoter also additionally was demonstrated to tolerate a TC insertion at +61 (thereby effectively introducing an internal ClaI site within the promoter region). It is likely that any combination of two nucleotides (i.e., AA, CC, TT, GG, AC, AT, AG, CA, CT, CG, TA, TG, GA, GC or GT) could be introduced at the +61 position, without impairing the active status of the promoter—wherein the active status of the promoter is based on a determination of the promoter's ability to enable expression of a coding region of interest that is expressible in a transformed yeast cell, when the promoter region is operably linked to the coding region.

In addition to tolerating various substitutions and insertions within SEQ ID NO:2, the GPD-NcoI*-ClaI*-C-60 (SEQ ID NO:7) also demonstrated that the wildtype promoter set forth as SEQ ID NO:2 could be truncated. Deleting the region defined as +1 to +60 bp of SEQ ID NO:2 resulted in the active mutant promoter described herein as GPD-NcoI*-ClaI*-C-60, which corresponds to bases 61 to 968 of SEQ ID NO:2 (i.e., also corresponding to the −908 to −1 region of the Yarrowia lipolytica gpd gene.

Based on the results described above, one of skill in the art will therefore recognize that Yarrowia GPD promoter regions corresponding to (at least) the −908 to −1 region, the −909 to −1 region, the −910 to −1 region, the −911 to −1 region, the −912 to −1 region, −913 to −1 region, the −914 to −1 region, the −915 to −1 region, the −916 to −1 region, the −917 to −1 region, the −918 to −1 region, the −919 to −1 region, −920 to −1 region, the −921 to −1 region, the −922 to −1 region, the −923 to −1 region, the −924 to −1 region, the −925 to −1 region, the −926 to −1 region, −927 to −1 region, the −928 to −1 region, the −929 to −1 region, the −930 to −1 region, the −931 to −1 region, the −932 to −1 region, −933 to −1 region, the −934 to −1 region, the −935 to −1 region, the −936 to −1 region, the −937 to −1 region, the −938 to −1 region, the −939 to −1 region, −940 to −1 region, the −941 to −1 region, the −942 to −1 region, the −943 to −1 region, the −944 to −1 region, the −945 to −1 region, the −946 to −1 region, −947 to −1 region, the −948 to −1 region, the −949 to −1 region, the −950 to −1 region, the −951 to −1 region, the −952 to −1 region, −953 to −1 region, the −954 to −1 region, the −955 to −1 region, the −956 to −1 region, the −957 to −1 region, the −958 to −1 region, the −959 to −1 region, −960 to −1 region, the −961 to −1 region, the −962 to −1 region, the −963 to −1 region, the −964 to −1 region, the −965 to −1 region, the −966 to −1 region and the −967 to −1 region upstream of the Yarrowia gpd gene will be active. Thus, any of these promoter regions could be used for expression of a coding region of interest in a Yarrowia host cell.

### Example 4

### Use Of Select Modified Yarrowia GPD Promoters In Yarrowia lipolytica Strain Y8672, Producing 61.8% Eicosapentaenoic Acid Of Total Fatty Acids [“TFAs”]

The present Example describes the construction of strain Y8672, derived from Yarrowia lipolytica ATCC #20362, capable of producing about 61.8% EPA relative to the total lipids via expression of a delta-9 elongase/delta-8 desaturase pathway. The development of strain Y8672 (FIG. 5) required the construction of strains Y2224, Y4001, Y4001 U, Y4036, Y4036U, L135, L135U9, Y8002, Y8006U6, Y8069, Y8069U, Y8145, Y8145U, Y8259, Y8259U, Y8367 and Y8367U.

The final genotype of strain Y8672 with respect to wild type Yarrowia lipolytica ATCC #20362 included four chimeric genes described as: GPD::ME3S::Pex20, GPD::FmD12::Pex20, GPD::EaD8S::Pex16 (2 copies) and GPD::YICPT1::Aco. The supposed “GPD” promoter in each of these cassettes corresponds to one of the modified Yarrowia GPD promoters described in Example 3 (supra), as summarized in Table 4 and described in additional detail below.

TABLE 4
Use Of Modified Yarrowia GPD Promoters In Genetically
Engineered Strains of Yarrowia lipolytica Producing PUFAs

| Plasmid | | Promoter | |
|---|---|---|---|
| (SEQ ID NO) | Promoter | SEQ ID NO | Chimeric Gene |
| pZKLeuN-29E3 | GPD-Ncol*- | SEQ ID NO: 5 | GPD::FmD12::Pex20 |
| (SEQ ID NO: 10) | Clal*-C | | |
| pZKL2-5m89C | GPD-TC-Ncol*- | SEQ ID NO: 6 | GPD::YICPT1::Aco |
| (SEQ ID NO: 11) | Clal*-C | | |
| pZP2-85m98F | GPD-Ncol*- | SEQ ID NO: 7 | GPD::EaD8S::Pex16 |
| (SEQ ID NO: 12) | Clal*-C-60 | | |
| pZSCP-Ma83 | GPD-Ncol*- | SEQ ID NO: 7 | GPD::EaD8S::Pex16 |
| (SEQ ID NO: 13) | Clal*-C-60 | | |
| pZSCP-Ma83 | GPD-Ncol*- | SEQ ID NO: 5 | GPD::ME3S::Pex20 |
| (SEQ ID NO: 13) | Clal*-C | | |

### Generation of Strain Y4001 to Produce About 17% EDA of TFAs

The generation of strain Y4001 is described in Example 7 of Intl. App. Pub. No. WO 2008/073367 and in the General Methods of U.S. Pat. App. Pub. No. 2008-0254191, hereby incorporated herein by reference. Briefly, construct pZKLeuN-29E3 (SEQ ID NO:10; FIG. 6A) was integrated into the Leu2 loci of strain Y2224 (a FOA resistant mutant from an autonomous mutation of the Ura3 gene of wildtype Yarrowia strain ATCC #20362). Although construct pZKLeuN-29E3 comprised four chimeric genes (i.e., a delta-12 desaturase, a C_{16/18} elongase and two delta-9 elongases), the chimeric GPD::FmD12::Pex20 gene is of relevance to the present discussion. Specifically, the FmD12 gene (labeled as “F.D12” in the Figure and corresponding to a codon-optimized delta-12 desaturase gene derived from Fusarium moniliforme [U.S. Pat. No. 7,504,259]) was operably linked to a “GPD” promoter sequence that corresponds to GPD-NcoI*-ClaI*-C (SEQ ID NO:5) (Example 3).

### Generation of Strain Y8145 to Produce About 48.5% EPA of TFAs

The generation of strain Y4036U is described in Example 7 of Intl. App. Pub. No. WO 2008/073367 and in the General Methods of U.S. Pat. App. Pub. No. 2008-0254191, hereby incorporated herein by reference. Briefly, following the isolation of strain Y4001 U, having a Leu- and Ura- phenotype, construct pKO2UF8289 was integrated into the native delta-12 desaturase loci of strain Y4001 U1. This resulted in isolation of strain Y4036, producing about 18.2% DGLA of total lipids. Construct pKO2UF8289 comprised four chimeric genes (i.e., a delta-12 desaturase, one delta-9 elongase and two mutant delta-8 desaturases).

Following the isolation of strain Y4036U, having a Leu- and Ura- phenotype and described in Example 7 of Intl. App. Pub. No. WO 2008/073367 and in the General Methods of U.S. Pat. App. Pub. No. 2008-0254191 (hereby incorporated herein by reference), strains L135U9, Y8002, Y8006U6, Y8069, Y8069U and Y8145 were isolated, as described in U.S. patent application Ser. No. 12/814,815, filed Jun. 14, 2010 (E.I. duPont de Nemours & Co., Inc., Attorney Docket No. “CL4674USNA”, hereby incorporated herein by reference).

Briefly, however, construct pY157 was used to knock out the chromosomal gene encoding the peroxisome biogenesis factor 3 protein [peroxisomal assembly protein Peroxin 3 or “Pex3p”] in strain Y4036U, thereby producing strain L135. Strain L135U9 was then created to produce a Leu- and Ura- phenotype, and subsequently subjected to transformation with construct pZKSL-5S5A5 to result in isolation of strain Y8002, producing about 34% ARA of total lipids. Construct pZKSL-5S5A5 was designed to integrate three delta-5 desaturase genes into the Lys loci of strain L135U9. Then, construct pZP3-Pa777U (described in Table 9 of U.S. Pat. Appl. Pub. No. 2009-0093543-A1, hereby incorporated herein by reference) was designed to integrate three delta-17 desaturase genes into the Pox3 loci (GenBank Accession No. AJ001301) of strain Y8002, thereby resulting in isolation of strain Y8006, producing about 41% ARA of total lipids. Following the isolation of strain Y8006U6, having a Ura- phenotype, construct pZP3-Pa777U was integrated into the Yarrowia genome of strain Y8006U6. This resulted in isolation of strain Y8069, producing 37.5% EPA of total lipids.

Following isolation of strain Y8069U3, having a Ura- phenotype, construct pZKL2-5m89C (SEQ ID NO:11; FIG. 6B) was designed to integrate into the Lip2 loci (GenBank Accession No. AJ012632) of strain Y8069U3. This resulted in isolation of strain Y8145, producing about 48.5% EPA of total lipids. Although construct pZKL2-5m89C comprised chimeric genes encoding a delta-5 desaturase, a delta-9 elongase, a delta-8 desaturase, and a diacylglycerol cholinephosphotransferase gene [“CPT1 ”], the chimeric GPD::YICPT1::Aco gene is of relevance to the present discussion. Specifically, the Yarrowia lipolytica CPT1 gene (“YICPT1”; Intl. App. Pub. No. WO 2006/052870), was operably linked to a GPD promoter sequence that corresponds to GPD-TC-NcoI*-ClaI*-C (SEQ ID NO:6) (Example 3).

Generation of Y8367 Strain to Produce about 58.3% EPA of TFAs

The generation of strain Y8367 is described in U.S. patent application Ser. No. 12/814,815, filed Jun. 14, 2010 (E.I. du Pont de Nemours & Co., Inc., Attorney Docket No. “CL4674USNA”, hereby incorporated herein by reference). Briefly, following the isolation of strain Y8145U, having a Ura- phenotype, construct pZKL1-2SR9G85 was designed to integrate into the Lip1 loci (GenBank Accession No. Z50020) of strain Y8145U, resulting in isolation of strain Y8259, producing 53.9% EPA of total lipids. Construct pZKL1-2SR9G85 comprised chimeric genes encoding a DGLA synthase gene, a delta-12 desaturase and a delta-5 desaturase. Yarrowia lipolytica strain Y8259 was deposited with the American Type Culture Collection on May 14, 2009 and bears the designation ATCC PTA-10027.

Following the isolation of strain Y8259U, having a Ura- phenotype, construct pZP2-85m98F (SEQ ID NO:12; FIG. 7A) was designed to integrate into the Yarrowia Pox2 locus (GenBank Accession No. AJ001300) of strain Y8259U. This resulted in isolation of strain Y8367, producing about 58.3% EPA of total lipids. Although construct pZP2-85m98F comprised three chimeric genes (i.e., a delta-8 desaturase gene, a DGLA synthase gene, and a delta-5 desaturase gene), the chimeric GPD::EaD8S::Pex16 gene is of relevance to the present discussion. Specifically, the EaD8S gene, corresponding to a codon-optimized delta-8 desaturase gene derived from Euglena anabaena (U.S. Pat. No. 7,790,156), was operably linked to a “GPD” promoter sequence that corresponds to GPD-NcoI*-ClaI*-C-60 (SEQ ID NO:7) (Example 3).

Generation of Y8672 Strain to Produce about 61.8% EPA of TFAs

The generation of strain Y8672 is described in U.S. patent application Ser. No. 12/814,815, filed Jun. 14, 2010 [E.I. du Pont de Nemours & Co., Inc., Attorney Docket No. “CL4674USNA”, hereby incorporated herein by reference]. Briefly, following the isolation of strain Y8367U, having a Ura- phenotype, construct pZSCP-Ma83 (SEQ ID NO:13; FIG. 7B) was designed to integrate into the SCP2 loci (GenBank Accession No. XM_{—}503410) of strain Y8637U. This resulted in isolation of strain Y8672, producing about 61.8% EPA of total lipids. Although construct pZSCP-Ma83 comprised three chimeric genes (i.e., a delta-8 desaturase gene, a C_{16/18} elongase gene and a malonyl-CoA synthetase gene), both the chimeric GPD::EaD8S::Pex16 gene and chimeric GPD::ME3S::Pex20 gene are of relevance to the present discussion. Specifically, the EaD8S gene (supra) was operably linked to a GPD promoter sequence that corresponds to GPD-NcoI*-ClaI*-C-60 (SEQ ID NO:7) (Example 3). The ME3S gene, corresponding to a codon-optimized C_{16/18} elongase gene, derived from Mortierella alpina (U.S. Pat. No. 7,470,532), was operably linked to a GPD promoter sequence that corresponds to GPD-NcoI*-ClaI*-C (SEQ ID NO:5) (Example 3).

Thus, three different modified mutant Yarrowia GPD promoters derived from the exemplary 971 bp Yarrowia GPD promoter set forth as SEQ ID NO:2 (corresponding to the −968 to −1 upstream region of the gpd gene and the +1 to +3 region of the gpd gene [U.S. Pat. No. 7,259,255]) were utilized in various chimeric genes within strain Y8672, to enable expression of various PUFA biosynthetic pathway genes. These mutant promoters comprise various insertions, substitutions and regions upstream of the gpd gene, including the −908 to −1 region. More specifically, each of the modified Yarrowia GPD promoters utilized within pZKLeuN-29E3 (SEQ ID NO:10), pZKL2-5m89C (SEQ ID NO:11), pZP2-85m98F (SEQ ID NO:12) and pZSCP-Ma83 (SEQ ID NO:13) enabled successful expression of the coding region to which it was linked, upon expression in Yarrowia lipolytica. Thus, it was demonstrated herein that DNA fragments of altered sequence and diminished length may have promoter activity comparable to the promoter activity of the sequence set forth in SEQ ID NO:2; these constituted promoter regions of a Yarrowia gpd gene that differ from the promoter region set forth in SEQ ID NO:2.

### Example 5

### Sequence Analysis of Promoter Regions of a gpd Yarrowia Gene

The present Example describes the identification of a TATA-box within promoter regions of a gpd Yarrowia gene.

Specifically, the 5′ untranslated region of SEQ ID NO:1 was analyzed for the presence of a typical TATA box sequence. Nucleotides 1439-1445 of SEQ ID NO:1 (corresponding to the −87 to −81 region [FIG. 1]) are as follows: 5′-TATATAA-3′. This A/T-rich region was thus identified as a TATA-box, and it is expected that this is the location where the transciption initiation complex would form for DNA transcription. Based on the identification of the TATA-box, it is believed that the 87 base pair sequence (i.e., set forth as SEQ ID NO:16) spanning the region between the TATA-box at −87 to −81 of SEQ ID NO:1 up to the ‘ATG’ translation initiation codon of the gpd gene would be a suitable minimal promoter region for basal level transcription initiation.

What is claimed is:

1. A method for the expression of a coding region of interest in a transformed yeast cell comprising:
a) providing the transformed yeast cell having a chimeric gene, wherein the chimeric gene comprises:
(1) a promoter region of a gpd Yarrowia gene; and,
(2) the coding region of interest which is expressible in the yeast cell;
wherein the promoter region is operably linked to the coding region of interest; and,
b) growing the transformed yeast cell of step (a) under conditions whereby the chimeric gene of step (a) is expressed.

2. The method according to claim 1 wherein the promoter region of a gpd Yarrowia gene comprises SEQ ID NO:16,

3. The method according to claim 1 wherein the promoter region of a gpd Yarrowia gene is set forth in SEQ ID NO:15, wherein said promoter optionally comprises at least one modification selected from the group consisting of:
(a) a deletion at the 5′-terminus of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259 or 260 consecutive nucleotides, wherein the first nucleotide deleted is the thymine nucleotide [‘T’] at position 1 of SEQ ID NO:15;
(b) insertion of any two nucleotides [‘NN’] after the adenine [‘A’] nucleotide at position +160 and before the guanine [‘G’] nucleotide at position +161 of SEQ ID NO:15;
(c) insertion of a cytosine [‘C’] nucleotide at the 3′ end of SEQ ID NO:15 after the cytosine [‘C’] nucleotide at position +1068; and,
(d) any combination of part (a), part (b) and part (c) above.

4. The method according to claim 1 wherein the promoter region of a gpd Yarrowia gene is set forth in SEQ ID NO:14, wherein said promoter optionally comprises at least one modification selected from the group consisting of:
(a) a deletion at the 5′-terminus of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 consecutive nucleotides, wherein the first nucleotide deleted is the guanine nucleotide [‘G’] at position 1 of SEQ ID NO:14;
(b) insertion of a thymine nucleotide and a cytosine nucleotide [‘TC’] after the adenine [‘A’] nucleotide at position +60 and before the guanine [‘G’] nucleotide at position +61 of SEQ ID NO:14;
(c) insertion of any two nucleotides [‘NN’] after the adenine [‘A’] nucleotide at position +60 and before the guanine [‘G’] nucleotide at position +61 of SEQ ID NO:14;
(d) insertion of a cytosine [‘C’] nucleotide at the 3′ end of SEQ ID NO:14 after the cytosine [‘C’] nucleotide at position +968;
(e) any combination of part (a), part (b), part (c) and part (d) above.

5. The method according to claim 4 wherein the promoter region of a gpd Yarrowia gene is selected from the group consisting of SEQ ID NO:3, SEQ ID NO:5, SEQ ID NO:6 and SEQ ID NO:7.

6. The method according to claim 1 wherein the transformed yeast cell is an oleaginous yeast.

7. The method of claim 6, wherein the oleaginous yeast is a member of a genus selected from the group consisting of Yarrowia, Candida, Rhodotorula, Rhodosporidium, Cryptococcus, Trichosporon and Lipomyces.

8. The method according to claim 1 wherein the coding region of interest encodes a polypeptide, wherein the polypeptide is selected from the group consisting of: desaturases, elongases, acyltransferases, aminopeptidases, amylases, carbohydrases, carboxypeptidases, catalyases, cellulases, chitinases, cutinases, cyclodextrin glycosyltransferases, deoxyribonucleases, esterases, alpha-galactosidases, beta-galactosidases, glucoamylases, alpha-glucosidases, beta-glucanases, beta-glucosidases, invertases, laccases, lipases, mannosidases, mutanases, oxidases, pectinolytic enzymes, peroxidases, phospholipases, phosphotases, phytases, polyphenoloxidases, proteolytic enzymes, ribonucleases, transglutaminases and xylanases.

9. A method for the production of an omega-3 fatty acid or omega-6 fatty acid comprising:
a) providing a transformed oleaginous yeast comprising a chimeric gene, wherein the chimeric gene comprises:
i) a promoter region of a gpd Yarrowia gene; and,
ii) a coding region encoding at least one omega-3 fatty acid or omega-6 fatty acid biosynthetic pathway enzyme;
wherein the promoter region and the coding region are operably linked; and,
b) growing the transformed oleaginous yeast of step (a) under conditions whereby the at least one omega-3 fatty acid or omega-6 fatty acid biosynthetic pathway enzyme is expressed and the omega-3 fatty acid or the omega-6 fatty acid is produced; and,
c) optionally recovering the omega-3 fatty acid or the omega-6 fatty acid.

10. The method according to claim 9 wherein the promoter region of a gpd Yarrowia gene is set forth in SEQ ID NO:14, wherein said promoter optionally comprises at least one modification selected from the group consisting of:
a) a deletion at the 5′-terminus of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 consecutive nucleotides, wherein the first nucleotide deleted is the guanine nucleotide [‘G’] at position 1 of SEQ ID NO:14;
b) insertion of a thymine nucleotide and a cytosine nucleotide [‘TC’] after the adenine [‘A’] nucleotide at position +60 and before the guanine [‘G’] nucleotide at position +61 of SEQ ID NO:14;
c) insertion of any two nucleotides [‘NN’] after the adenine [‘A’] nucleotide at position +60 and before the guanine [‘G’] nucleotide at position +61 of SEQ ID NO:14;
d) insertion of a cytosine [‘C’] nucleotide at the 3′ end of SEQ ID NO:14 after the cytosine [‘C’] nucleotide at position +968;
e) any combination of part (a), part (b), part (c) and part (d) above.

11. The method according to claim 10 wherein the promoter region of a gpd Yarrowia gene is selected from the group consisting of SEQ ID NO:3, SEQ ID NO:5, SEQ ID NO:6 and SEQ ID NO:7.

12. The method according to claim 9 wherein the coding region encoding at least one omega-3 fatty acid or omega-6 fatty acid biosynthetic pathway enzyme is selected from the group consisting of: desaturases and elongases.

13. The method according to claim 12 wherein the desaturase is selected from the group consisting of: delta-9 desaturase, delta-8 desaturase, delta-12 desaturase, delta-6 desaturase, delta-5 desaturase, delta-17 desaturase, delta-15 desaturase and delta-4 desaturase and the elongase is selected from the group consisting of: a delta-9 elongase, a C_{14/16} elongase, a C_{16/18} elongase, a C_{18/20} elongase and a C_{20/22} elongase.

14. The method according to claim 9 wherein the oleaginous yeast is a member of a genus selected from the group of consisting of: Yarrowia, Candida, Rhodotorula, Rhodosporidium, Cryptococcus, Trichosporon and Lipomyces.

15. The method according to claim 14 wherein the oleaginous yeast is Yarrowia lipolytica.

16. The method according to claim 9 wherein the omega-3 fatty acid or the omega-6 fatty acid is selected from the group consisting of: linoleic acid, gamma-linolenic acid, eicosadienoic acid, dihomo-gamma-linolenic acid, arachidonic acid, alpha-linoleic acid, stearidonic acid, eicosatrienoic acid, eicosatetraenoic acid, eicosapentaenoic acid, docosatetraenoic acid, omega-6 docosapentaenoic acid, omega-3 docosapentaenoic acid and docosahexaenoic acid.

17. An isolated nucleic acid molecule comprising a promoter region of a gpd Yarrowia gene as set forth in SEQ ID NO:15, wherein said promoter optionally comprises at least one modification selected from the group consisting of:
(a) a deletion at the 5′-terminus of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259 or 260 consecutive nucleotides, wherein the first nucleotide deleted is the thymine nucleotide [‘T’] at position 1 of SEQ ID NO:15;
(b) insertion of any two nucleotides [‘NN’] after the adenine [‘A’] nucleotide at position +160 and before the guanine [‘G’] nucleotide at position +161 of SEQ ID NO:15;
(c) insertion of a cytosine [‘C’] nucleotide at the 3′ end of SEQ ID NO:15 after the cytosine [‘C’] nucleotide at position +1068; and,
(d) any combination of part (a), part (b) and part (c) above.

18. An isolated nucleic acid molecule comprising a promoter region of a gpd Yarrowia gene as set forth in SEQ ID NO:14, wherein said promoter optionally comprises at least one modification selected from the group consisting of:
(a) a deletion at the 5′-terminus of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 consecutive nucleotides, wherein the first nucleotide deleted is the guanine nucleotide [‘G’] at position 1 of SEQ ID NO:14;
(b) insertion of a thymine nucleotide and a cytosine nucleotide [‘TC’] after the adenine [‘A’] nucleotide at position +60 and before the guanine [‘G’] nucleotide at position +61 of SEQ ID NO:14;
(c) insertion of any two nucleotides [‘NN’] after the adenine [‘A’] nucleotide at position +60 and before the guanine [‘G’] nucleotide at position +61 of SEQ ID NO:14;
(d) insertion of a cytosine [‘C’] nucleotide at the 3′ end of SEQ ID NO:14 after the cytosine [‘C’] nucleotide at position +968;
(e) any combination of part (a), part (b), part (c) and part (d) above.

19. The isolated nucleic acid molecule of claim 18 selected from the group consisting of SEQ ID NO:3, SEQ ID NO:5, SEQ ID NO:6 and SEQ ID NO:7.

20. An isolated nucleic acid molecule comprising a promoter region of a gpd Yarrowia gene comprising SEQ ID NO:16.
